(19) United States
(12) Patent Application Publication
(10) Pub. No.: US 2012/0046461 A1
(43) Pub. Date: Feb. 23, 2012

(54) STEROIDAL ANTI-HORMONE HYBRIDS

(75) Inventor: Robert N. Hanson, Newton, MA (US)

(73) Assignee: Northeastern University, Boston, MA (US)

(21) Appl. No.: 13/145,965

(22) PCT Filed: Jan. 25, 2010
(86) PCT No.: PCT/US10/21987

§ 371 Date: Nov. 9, 2011

Related U.S. Application Data
(60) Provisional application No. 61/146,934, filed on Jan. 23, 2009.

(51) Int. Cl. C07J 43/00

(52) U.S. Cl. 540/108

(57) ABSTRACT

Disclosed are novel compounds and compositions for inhibition of androgen and estrogen receptor signaling, methods for inhibiting androgen signaling, methods for inhibiting estrogen signaling, methods for inhibiting the interaction between a co-regulatory protein and an androgen or estrogen receptor, and methods for treating cancer.

### PRIORITY claim

This disclosure claims the benefit of U.S. Provisional Application No. 61/146,934, filed Jan. 23, 2009. The entire disclosure of that application is relied on and incorporated into this application by reference.

### FEDERALLY SPONSORED RESEARCH OR DEVELOPMENT

This invention was sponsored through grants from the National Institutes of Health [PHS 1R01CA81049 (R.N.H.) and PHS 1R01CA 37799 (R.B.H.)], the U.S. Army Breast Cancer Research Program [DAMD 17-00-1-00384 and W81HWO410544(R.N.H.)] and, thus, the U.S. government has certain rights in this application.

### FIELD

The present disclosure relates to the fields of medicinal chemistry and biology.

### BACKGROUND

Researchers have been trying to develop new chemotherapeutic agents by coupling two biologically active compounds to make a single hybrid agent. In the field of hormone responsive breast cancer, for example, this involves linking a potent estrogen receptor targeting agent to a second component, such as an anti-metabolite, intercalating agent, anti-mitotic, alkylating agent or metal chelating group. Usually, the resultant product has proved to be less effective at each of its targets than the separate, individual components, themselves.

There are numerous difficulties associated with designing bi-functional hybrid drugs. In the case of steroid receptor-targeted hybrids, there are problems related to an over-reliance on chemical transformations of readily available materials or easily modified sites on those materials to prepare the target compounds. While attachment of functional groups at the 3-, 6-, 17α-, and 17β-positions can be done, such modifications impair binding to the receptor. Introduction of substituents at the 7α-position, such as those found in the anti-estrogen Faslodex (also known as fulvestrant), requires more steps. However, crystal structures of complexes of similarly substituted ligands with ERα-LBD (estrogen receptor alpha-ligand binding domain) indicate that the steroidal scaffold is rotated around the 3-17 axis, and, unfortunately, there is disorder associated with helix-12.

### SUMMARY

It has been discovered that the 11β position on a steroid structure is the position where modifications are well tolerated by the receptor. This discovery has been exploited to develop the present disclosure, which, in part, is directed to novel steroidal compounds, novel derivatized quinone antiobiotic compounds, novel derivatized tyrosine kinase inhibitors, and novel hybrids that combine the novel steroidal compounds and derivatized biologically active compounds through ligation.

One aspect of the present disclosure is directed to novel compounds of Formula (I),

S′—B′ Formula (I)

wherein:
S′ is S-L′, where S is an 11β-substituted steroid; B′ is B-L″, where B is a biologically active moiety; and L′ and L″ are each a half-linker that together form L, a linker.

In some embodiments, S is an anti-estrogen steroid. In other embodiments, S is estradiol.

In yet other embodiments, S is an anti-androgen steroid. In additional embodiments, S is an anti-progestin steroid. In further embodiments, S is an anti-glucocorticoid steroid. In further embodiments, S is a compound having the structure of Formula (III), described below.

In some embodiments, B is an antiobiotic. In additional embodiments, B is a dye for photodynamic therapy. In some embodiments, B is a nitroxyl group. In still other embodiments, B is a tyrosine kinase inhibitor.

Another aspect of the present disclosure is directed to novel compounds of Formula (Ia),

S′-A′ Formula (Ia)

wherein:
S′ is S-L′, where S is an 11β-substituted steroid; A′ is A-L″, where A is a quinone antibiotic; and L″ are each a half-linker that together form L, a linker.

In some embodiments, S is an anti-estrogen steroid. In further embodiments, S is estradiol.

In other embodiments, S is an anti-androgen steroid. In additional embodiments, S is an anti-progestin steroid. In some embodiments, S is an anti-glucocorticoid steroid.

In further embodiments, S is a compound having the structure of Formula (III), described below.

In some embodiments, quinone antibiotic A comprises the structure

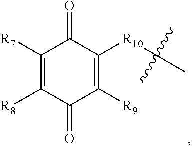

wherein R₇, R₈, and R₉ are each independently H, alkyl, cycloalkyl, aralkyl, alkoxy, aryl, heterocycle, amine, or halogen; and

R₁₀ is C, S, N, or O.

In some embodiments, A is mitomycin C. In other embodiments, A is geldanamycin.

A further aspect of this disclosure is directed to compounds having Formula (II),

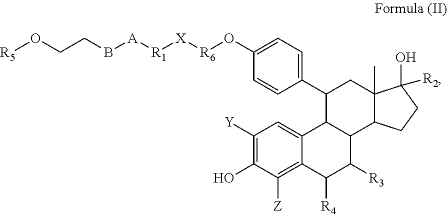

wherein R₁ is an oligoethylene glycol having from 1-10 units;

R₂ is H, C₂-C₆ alkyl ethenyl, ethynyl, haloethenyl, alkylthioethenyl, alkylselenoethenyl, arylthioethenyl, arylselenoethenyl, or aryl vinyl wherein the arylvinyl may have up to four substituents and the substituents may be alkyl, aryl, or fluoroalkyl;

R₃ is a C₂-C₆-alkyl, aralkyl, hydroxyl, ketone, or ether;

R₄ is a C₂-C₆-alkyl, aralkyl, hydroxyl, ketone, or ether;

R₅ is a quinone-containing antiobiotic, a tyrosine kinase inhibitor, an alkylating agent, a fluorescent dye, a NIR dye, an MR imaging agent, a positron-emitting group, or a photon-emitting group;

R₆ is an alkyl or cycloalkyl; A-B is a linker and contains a triazole, a thiolated maleimide, an amide, a urea, a thiourea, a squaramide, an aminoalkyl ether, a thioalkyl ether, or an alkenyl;

X is O, NH, N-alkyl, N-cycloalkyl, N-aralkyl, or S; and

Y and Z are each independently H, F, Cl, Br, I, C₁-C₆-alkyl, hydroxyalkyl, alkoxyalkyl, NO₂, or NH₂.

Yet a further aspect of this disclosure is directed to novel derivatized anti-estrogen compounds having Formula (III),

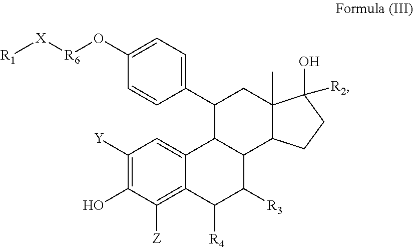

wherein R₁ is an oligoethylene glycol having from 1-10 units; R₂ is H, C₂-C₆-alkyl, ethenyl, ethynyl, haloethenyl, alkylthioethenyl, alkylselenoethenyl, arylthioethenyl, arylselenoethenyl, or aryl vinyl wherein the arylvinyl may have up to four substituents and the substituents may be alkyl, aryl, or fluoroalkyl;

R₃ is a C₂-C₆-alkyl, aralkyl, hydroxyl, ketone, or ether;

R₄ is a C₂-C₆-alkyl, aralkyl, hydroxyl, ketone, or ether;

R₆ is an alkyl or cycloalkyl;

X is O, NH, N-alkyl, N-cycloalkyl, N-aralkyl, or S; and Y and Z are each independently H, F, Cl, Br, I, C₁-C₆-alkyl, hydroxyalkyl, alkoxyalkyl, NO₂, or NH₂.

An additional aspect of this disclosure is directed to novel derivatized tyrosine kinase inhibitors of Formula (IV),

T-L′ Formula (IV)

wherein T is:

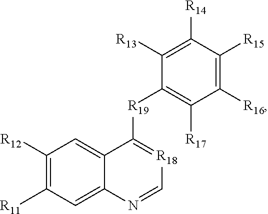

wherein:

R₁₁, R₁₂, R₁₃, R₁₄, R₁₅, R₁₆, and R₁₇ are each independently H, alkyl, cycloalkyl, aralkyl, alkoxy, aryl, heterocycle, amine, or halogen;

R₁₉ is NH or O;

R₁₈ is N or CH; and

L′ is a half-linker or linker and is attached to T through R₁₁, R₁₂, R₁₄, R₁₅, or R₁₆.

Another aspect of this disclosure is directed to derivatized quionone antibiotics according to Formula (V),

A-L′ Formula (V),

wherein A is a quinone antibiotic and L′ is a half-linker or linker.

Quinone antibiotic A comprises the structure

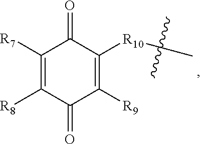

wherein R₇, R₈, and R₉ are each independently H, alkyl, cycloalkyl, aralkyl, alkoxy, aryl, heterocycle, amine, or halogen; and

R₁₀ is C, S, N, or O.

Yet another aspect of this disclosure is directed to a method of inhibiting cell proliferation in cancer cells. In this method, a sample containing a cancer cell is contacted with a compound of Foimula (I), (Ia), (II), (III), (IV), or (V) or a pharmaceutically acceptable salt, hydrate, solvate, tautomer, or prodrug of Formula (I), (Ia), (II), (III), (IV), or (V). Then, the proliferative state of the cell in the sample is monitored. Cell stasis or death detected in the sample indicates the inhibition of cell proliferation.

Another aspect of this disclosure is directed to a method of treating cancer in a subject. In this method, a subject is administered a therapeutically effective amount of a pharmaceutical formulation comprising the compound of Formula (I), (Ia), (II), (III) or a pharmaceutically acceptable salt, hydrate, solvate, tautomer, or prodrug of Formula (I), (Ia), (II), (III), (IV), or (V).

In some embodiments, the cancer is prostate cancer. In other embodiments, the cancer is breast cancer.

Another aspect of the disclosure is directed to a method of disrupting estrogen signaling. In this method a sample is obtained that contains an estrogen receptor and a co-regulatory protein. The sample is contacted with a compound of Formula (I), (Ia), (II), (III), (IV), or (V), or a pharmaceutically acceptable salt, hydrate, solvate, tautomer, or prodrug of Formula (I), (Ia), (II), (III). The sample is monitored for formation of an amount of co-regulatory protein-estrogen receptor complex formed is detected. Then, the amount of complex formed in this sample is compared to an amount of complex formed in a control sample that does not contain the compound of Formula (I), (Ia), (II), (III), (IV), or (V). The estrogen signaling is disrupted when the amount of complex in the sample is detectably less than the amount of complex formed in the control sample.

### BRIEF DESCRIPTION OF THE DRAWINGS

The foregoing and other objects of this disclosure, the various features thereof, as well as the invention itself may be more fully understood from the following description, when readtogether with the accompanying drawings in which:

FIG. 1A is a graphic representation of the results of the assay in Ishikawa cells assay;

FIG. 1B is a graphic representation of the results of the stimulation of alkaline phosphatase in Ishikawa cells assay;

FIG. 1C is a graphic representation of the results of the inhibition in the alkaline phosphatase in Ishikawa cells;

FIG. 2 is a graphic representation of the results of the effects of the mitomycin moiety on cellular proliferation of MCF-7 (ER-positive) and MDA-231 (ER-negative) breast cancer cell lines;

FIGS. 3A-B are the crystal structures of anti-estrogens bound to the ERα-LBD used in biochemical studies with MMC;

FIG. 4 is a graphic representation of the results of the inhibition of cell proliferation assay in MCF-7 an dMDA-MB-231 cells;

FIG. 5 is a graphic representation of the results of cytotoxicity assay in MCF-7 an dMDA-MB-231 cells; and

FIG. 6. is a graphic representation of the EPR spectra of 3-8 unbound and bound to ERα-LBD;

### DESCRIPTION

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure.

This disclosure relates to novel compounds, pharmaceutical compositions comprising these compounds, methods for inhibiting androgen signaling, methods for inhibiting estrogen signaling, methods for inhibiting glucocorticoid signaling, methods for inhibiting the interaction between a co-regulatory protein and an androgen or estrogen receptor, methods of inhibiting cancer cell proliferation, and methods for treating cancer.

### 1. DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

The terms “compound” and “compounds” as used in this disclosure refer to the compounds of this disclosure and any and all possible isomers, stereoisomers, enantiomers, diastereomers, tautomers, phaimaceutically acceptable salts, and solvates thereof.

In general, the compositions of the disclosure can be alternately formulated to comprise, consist of, or consist essentially of, any appropriate components disclosed in this disclosure. The compositions of the disclosure can additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

The articles “a” and “an” are used in this disclosure to refer to one or more than one (i.e., to at least one) of the grammatical object of the article. By way of example, “an element” means one element or more than one element.

The term “or” is used in this disclosure to mean, and is used interchangeably with, the term “and/or,” unless indicated otherwise.

The term “about” is used in this disclosure to mean a value −or +20% of a given numerical value. Thus, “about 60%” means a value between 60-20% of 60 and 60+20% of 60 (i.e., between 48% and 72%).

The terms “alkyl” and unless otherwise specifically defined, refer to a straight or branched chain alkane (hydrocarbon) radical, which may be fully saturated, mono- or polyunsaturated, and can include divalent radicals, having from 1 to about 10 carbon atoms. Examples of saturated hydrocarbon radicals include, but are not limited to, groups such as methyl (Me), ethyl (Et), n-propyl, isopropyl, n-butyl, t-butyl, isobutyl, sec-butyl, homologs and isomers of, for example, n-pentyl, n-hexyl, n-heptyl, n-octyl, 1,1-dimethyl-heptyl, 1,2-dimethyl-heptyl, and the like. An unsaturated hydrocarbon group includes one or more double bonds, triple bonds or combinations thereof. Examples of unsaturated alkyl groups include, but are not limited to, vinyl, propenyl, crotyl, 2-isopentenyl, allenyl, butenyl, butadienyl, pentenyl, pentadienyl, 3-(1,4-pentadienyl), hexenyl, hexadienyl, ethynyl, propynyl, butynyl, and higher homologs and isomers. The term refers to an alkyl having from 1 to about m carbon atoms. The alkyl group may be optionally substituted with one or more substituents, e.g., 1 to 4 substituents, at any available point of attachment, as defined below.

The term “aralkyl”, unless otherwise specifically defined, refers to an alkyl group where an H has been replaced with an aryl group.

The term “aryl”, unless otherwise specifically defined, refers to cyclic, aromatic hydrocarbon groups that have 1 to 5 aromatic rings, including monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The aryl group may be optionally substituted by one or more substituents, e.g., 1 to 5 substituents, at any point of attachment. In addition to the substituents described under the definition of “substituted,” other exemplary substituents include, but are not limited to, nitro, cycloalkyl or substituted cycloalkyl, cycloalkenyl or substituted cycloalkenyl, cyano, alkyl, fused cyclic groups, fused cycloalkyl, fused cycloalkenyl, fused heterocycle, and fused aryl, and those groups recited above as exemplary alkyl substituents. The substituents can themselves be optionally substituted.

The term “cycloalkyl”, unless otherwise specifically defined, refers to a saturated or partially saturated cyclic hydrocarbon group containing from 1 to 4 rings and 3 to 8 carbons per ring, including, for example, 4 to 7 membered monocyclic groups, 7 to 12 membered bicyclic groups, or 8 to 16 membered tricyclic ring systems, polycyclic groups, or bridged systems. Exemplary groups include, but are not limited to, cyclopropyl, cyclobutyl, cyclopentyl, cyclohexyl, cycloheptyl, adamantyl, cyclobutenyl, cyclopentenyl, and cyclohexenyl, etc. The cycloalkyl group may be optionally substituted with one or more substituents, e.g., 1 to 5 substituents, at any available point of attachment. In addition to the substituents described under the definition of “substituted,” other exemplary substituents include, but are not limited to, nitro, cyano, alkyl, Spiro attached or fused cyclic substituents, spiro-attached cycloalkyl, spiro-attached cycloalkenyl, Spiro-attached heterocycle, fused cycloalkyl, fused cycloalkenyl, fused heterocycle, fused aryl, and those groups recited above as exemplary alkyl substituents. The substituents can themselves be optionally substituted.

The term “adamantyl”, unless otherwise specifically defined, includes, but is not limited to, 1 adamantyl, 2 adamantyl, and 3 adamantyl. The adamantyl group may be optionally substituted with the groups recited as exemplary cycloalkyl substituents as well as the substituents described under the definition of “substituted.”

The term “halogen” as used herein refers to fluorine, chlorine, bromine, and iodine.

The terms “heterocycle” and “heterocyclic”, unless otherwise specifically defined, refer to fully saturated, or partially or fully unsaturated, including aromatic (i.e., “heteroaryl”) cyclic groups (for example, 4 to 7 membered monocyclic, 7 to 12 membered bicyclic, or 8 to 16 membered tricyclic ring systems) which have at least one heteroatom in at least one carbon atom-containing, ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached to the remainder of the molecule at any heteroatom or carbon atom of the ring or ring system. Exemplary monocyclic heterocyclic groups include, but are not limited to, azetidinyl, pyrrolidinyl, pyrrolyl, pyrazolyl, oxetanyl, dioxanyl, dioxolanyl, oxathiolanyl, pyrazolinyl, imidazolyl, imidazolinyl, imidazolidinyl, oxazolyl, oxazolidinyl, isoxazolinyl, isoxazolyl, thietanyl, azetidine, diazetidine, thiolanyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, purinyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include, but are not limited to, indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include, but are not limited to, carbazolyl, benzidolyl, phenanthrolinyl, acridinyl, phenanthridinyl, xanthenyl, and the like.

A heterocyclic group may be optionally “substituted” with one or more substituents, e.g., 1 to 5 substituents, at any available point of attachment. In addition to the substituents described under the definition of “substituted,” other exemplary substituents include, but are not limited to, cycloalkyl or substituted cycloalkyl, cycloalkenyl or substituted cycloalkenyl, nitro, oxo (i.e., ═O), cyano, alkyl or substituted alkyl, spiro-attached or fused cyclic substituents at any available point or points of attachment, spiro-attached cycloalkyl, spiro-attached cycloalkenyl, Spiro-attached heterocycle (excluding heteroaryl), fused cycloalkyl, fused cycloalkenyl, fused heterocycle, fused aryl, and the like. The substituents can themselves be optionally substituted.

The term “substituted” means substituted by a below-described substituent group in any possible position. Substituent groups for the above moieties useful in this disclosure are those groups that do not significantly diminish the biological activity of the disclosed compound. Substituent groups that do not significantly diminish the biological activity of the disclosed compound include, but are not limited to, H, halogen, N₃, NCS, CN, NO₂, NX1X2, OX3, C(X3)₃, OAc, O-acyl, O-aroyl, NH-acyl, NH-aroyl, NHCOalkyl, CHO, C(halogen)₃, Ph, OPh, CH₂Ph, OCH₂Ph, COOX3, SO₃H, PO₃H₂, SO₂NX1X2, CONX1X2, alkyl, alcohol, alkoxy, dioxolanyl, alkylmercapto, dithiolanyl, dithianyl, alkylamino, dialkylamino, sulfonamide, thioalkoxy or methylene dioxy when the substituted structure has two adjacent carbon atoms, wherein X1 and X2 each independently comprise H or alkyl, and X3 comprises H, alkyl, hydroxyloweralkyl. Unless otherwise specifically limited, a substituent group may be in any possible position.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The phrase “pharmaceutically acceptable” is employed in this disclosure to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “salt” or “salts”, as employed in this disclosure, denote acidic and/or basic salts formed with inorganic and/or organic acids and bases.

The term “treating” with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject. The actual amount which comprises the “effective amount” or “therapeutically effective amount” will vary depending on a number of conditions including, but not limited to, the particular disorder being treated, the severity of the disorder, the size and health of the patient, and the route of administration. A skilled medical practitioner can readily determine the appropriate amount using methods known in the medical arts.

As used in this disclosure, the term “subject” includes, without limitation, a human or an animal. Exemplary animals include, but are not limited to, mammals such as mouse, rat, guinea pig, dog, cat, horse, cow, pig, monkey, chimpanzee, baboon, or rhesus monkey.

The terms “administer”, “administering”, or “administration” as used in this disclosure refer to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a compound of Formula (I).

The terms “isolated” and “purified” as used in this disclosure refer to a component separated from other components of a reaction mixture or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt of the compound by weight of the isolate.

The term “tautomer” as used in this disclosure refers to compounds produced by the phenomenon wherein a proton of one atom of a molecule shifts to another atom. (March, Advanced Organic Chemistry. Reactions, Mechanisms and Structures, 4th Ed., John Wiley & Sons, pp. 69-74 (1992)).

The following abbreviations are used in this disclosure and have the following definitions: DMF is dimethylformamide; DMSO is dimethylsulfoxide; THF is tetrahydrofuran; “min” is minute or minutes; is hour or hours; and “RT” is room temperature.

### 2. COMPOUNDS

This disclosure provides novel bifunctional compounds according to Formula (I), in which one of the functional groups is an 11β-substituted steroid and the other group is a biologically active moiety:

S′—B′ Formula (I)

wherein:
S′ is S-L′, where S is an 11β-substituted steroid; B′ is B-L″, where B is a biologically active moiety; and L′ and L″ are each a half-linker that together form L, a linker.

The steroid component S can be anti-estrogen, anti-androgen, anti-progestin, or anti-glucocorticoid with additional substituents at the 1, 2, 4, 7, 11, 16 or 17-positions. The 11-β-position is substituted with an aromatic group which may also be substituted.

The biologically active moiety B can be any biologically active moiety to which a half-linker can be attached without interfering with the moiety's biological activity. For example, the biologically active moiety B can be an antibiotic, a tyrosine kinase inhibitor, an alkylating agent, a fluorescent dye, a NIR dye, an MR imaging agent, a positron-emitting group, or a photon-emitting group, or an intercalating agents.

The 11β-substituted steroid and the biologically active moiety are connected by linker L. Linker L and “half-linkers” L′ and L″ can contain a triazole, a thiolated maleimide, an amide, a urea, a thiourea, a squaramide, an aminoalkyl ether, a thioalkyl ether, or an alkenyl. To synthesize the hybrid compounds, both the steroidal anti-hormone component and the biologically active component are terminally modified with half-linker L′ or L″. Half-linker L′ or L″ is introduced to sites that do not compromise the biological activity of either component by a process of total synthesis, generating new molecular entities. Half-linkers L′ or L″ may be oligoethylene glycols, polymethylene groups, polyamides, or combinations thereof, with a chemically reactive group at the terminus that can react in high efficiency with a complementary chemically reactive group at the terminus of a second half-linker.

The present disclosure further provides novel compounds of Formula (Ia),

S′-A′ Formula (Ia)

wherein:
S′ is S-L′, where S is an 11β-substituted steroid; A′ is A-L″, where A is a quinone antibiotic; and L′ and L″ are each a half linker that together form L, a linker.

Quinone antibiotic A can be, but is not limited to, mitomycin C or geldanamycin. Mitomycin C is useful for the treatment of advanced breast cancer. Mitomycin C belongs to the class of compounds that require metabolic activation, i.e, quinone reduction, prior to alkylation of the DNA. Mitomycin C also displays a degree of sequence selectivity based upon its molecular structure. In addition, structural modifications of the 7-amino group also retain anti-cancer and DNA alkylating activity.

This disclosure also provides compounds having Formula (II),

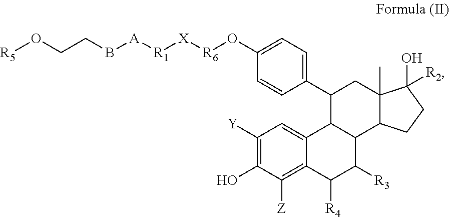

wherein:

R₁ is an oligoethylene glycol having from 1-10 units;
R₂ is H, C₂-C₆ alkyl, ethenyl, ethynyl, haloethenyl, alkylthioethenyl, alkylselenoethenyl, arylthioethenyl, arylselenoethenyl, or aryl vinyl wherein the arylvinyl may have up to four substituents and the substituents may be alkyl, aryl, or fluoroalkyl;
R₃ is a C₂-C₆-alkyl, aralkyl, hydroxyl, ketone, or ether;
R₄ is a C₂-C₆-alkyl, aralkyl, hydroxyl, ketone, or ether;
R₅ is a quinone-containing antiobiotic, a tyrosine kinase inhibitor, an alkylating agent, a fluorescent dye, a NIR dye, an MR imaging agent, a positron-emitting group, or a photon-emitting group;
R₆ is an alkyl or cycloalkyl;
A-B is a linker and contains a triazole, a thiolated maleimide, an amide, a urea, a thiourea, a squaramide, an aminoalkyl ether, a thioalkyl ether, or an alkenyl;
X is O, NH, N-alkyl, N-cycloalkyl, N-aralkyl, or S;
and Y and Z are each independently H, F, Cl, Br, I, C₁-C₆-alkyl, hydroxyalkyl, alkoxyalkyl, NO₂, or NH₂.

This disclosure also provides novel derivatized anti-estrogenic compounds having Formula (III),

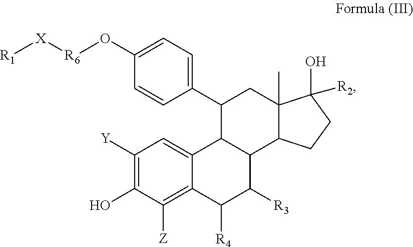

wherein R₁, R₂, R₃, R₁, R₆, X, Y, and Z are as defined above.

The position where modifications are best tolerated by the receptor is the 11β-site on the estradiol structure. Introduction of functional groups requires a multi-step synthetic sequence from the estradiol 3-methyl ether or 11-oxo-estradiol.

This disclosure further provides derivatized quionone antibiotics according to Formula (V),

A-L′ Formula (V),

wherein A is a quinone antibiotic and L′ is a half-linker or linker.

In this aspect, an antibiotic is combined with a half-linker to give a novel derivatized antibiotic. Quinone antibiotic A comprises the structure:

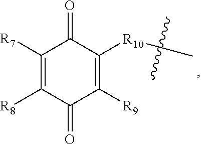

wherein R₇, R₈, and R₉ are each independently H, alkyl, cycloalkyl, aralkyl, alkoxy, aryl, heterocycle, amine, or halogen; and

R₁₀ is C, S, N, or O.

Derivatized Tyrosine Kinase Inhibitors and Tyrosine Kinase Inhibitor-Steroidal Hybrids

Growth factor receptor tyrosine kinase inhibitors (TKIs) have been used in various pharmaceutical compositions. For example, several agents in the 4-anilinoquinazoline subclass have been used for the treatment of patients with various forms of cancer, including ovarian and breast cancer. Erlotinib (Tarceva), Gefitinib (Iressa), and Vandetanib-ZD 6474 are useful representative 4-anilinoquinazoline TKIs.

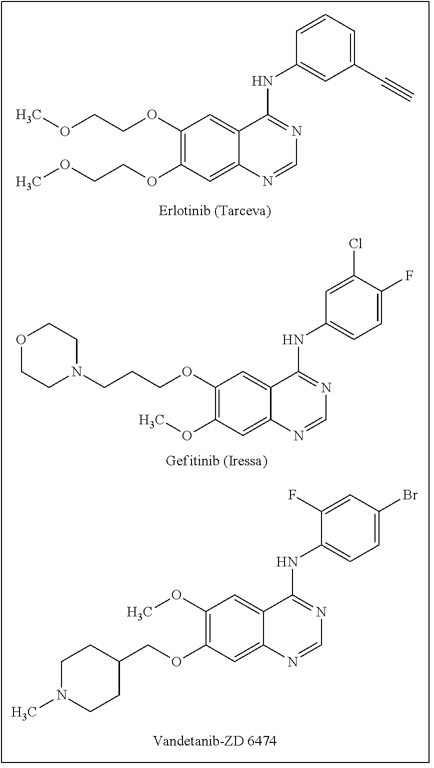

In this disclosure, the 3-position of the aniline group was modified to probe the ability of that binding pocket to tolerate a hydrophilic triethylene glycol linker as well as the terminal steroidal component. This disclosure also provides additional novel TKI derivatives having Formula (IV),

T-L′ Formula (IV)

wherein T is:

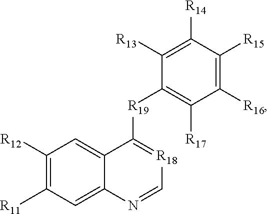

wherein:
R₁₁, R₁₂, R₁₃, R₁₄, R₁₅, R₁₆, and R₁₇ are each independently H, alkyl, cycloalkyl, aralkyl, alkoxy, aryl, heterocycle, amine, or halogen;
R₁₉ is NH or O;
R₁₈ is N or CH; and

L′ is a half-linker or linker and is attached to T through R₁₁, R₁₂, R₁₄, R₁₅, or R₁₆.

A hybrid compound was prepared in which an 11β-(azido-substituted phenyl) estradiol anti-estrogenic moiety was ligated to an analog of the ethynyl containing TKI, erlotinib (Tarceva) using the Huisgen [3+2] cycloaddition reaction. (Kolb, et al. (2001) Angewandte Chemie. Inter'l Ed., 40:2004-2021; Ramachary, et al. (2004) III Chemistry in Europe J., 10:5323-5331.)

To develop single molecule therapeutics with multiple activities incorporating a 4-anilinoquinazoline structure, a long and modified linker is used, based on the binding characteristics of the second molecular target (e.g., an estrogen receptor). Here, the two components are ligated using a “click chemistry” methodology such as the alkyne-azide Huisgen [3+2] cycloaddition reaction. The target used is shown in Scheme 1, where the propargyloxy group (selected as one coupling partner) can reside at either the 6- or 7-positions.

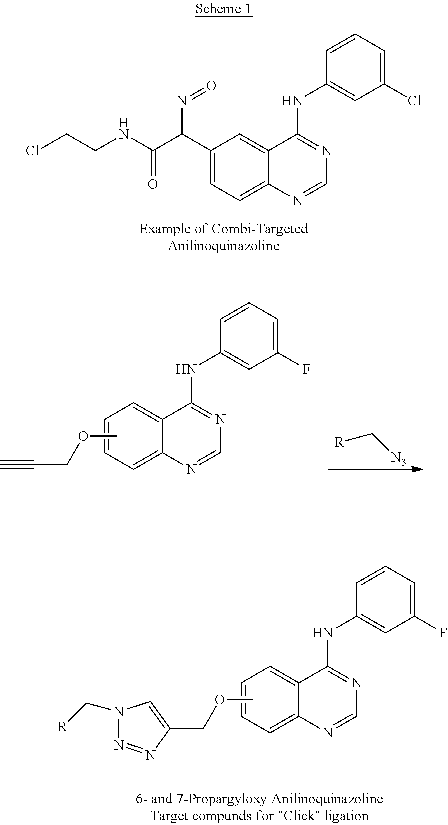

The following compounds are novel nonlimiting, representative, anti-estrogen/tyrosine kinase inhibitor hybrids useful in the treatment of NSCLC:

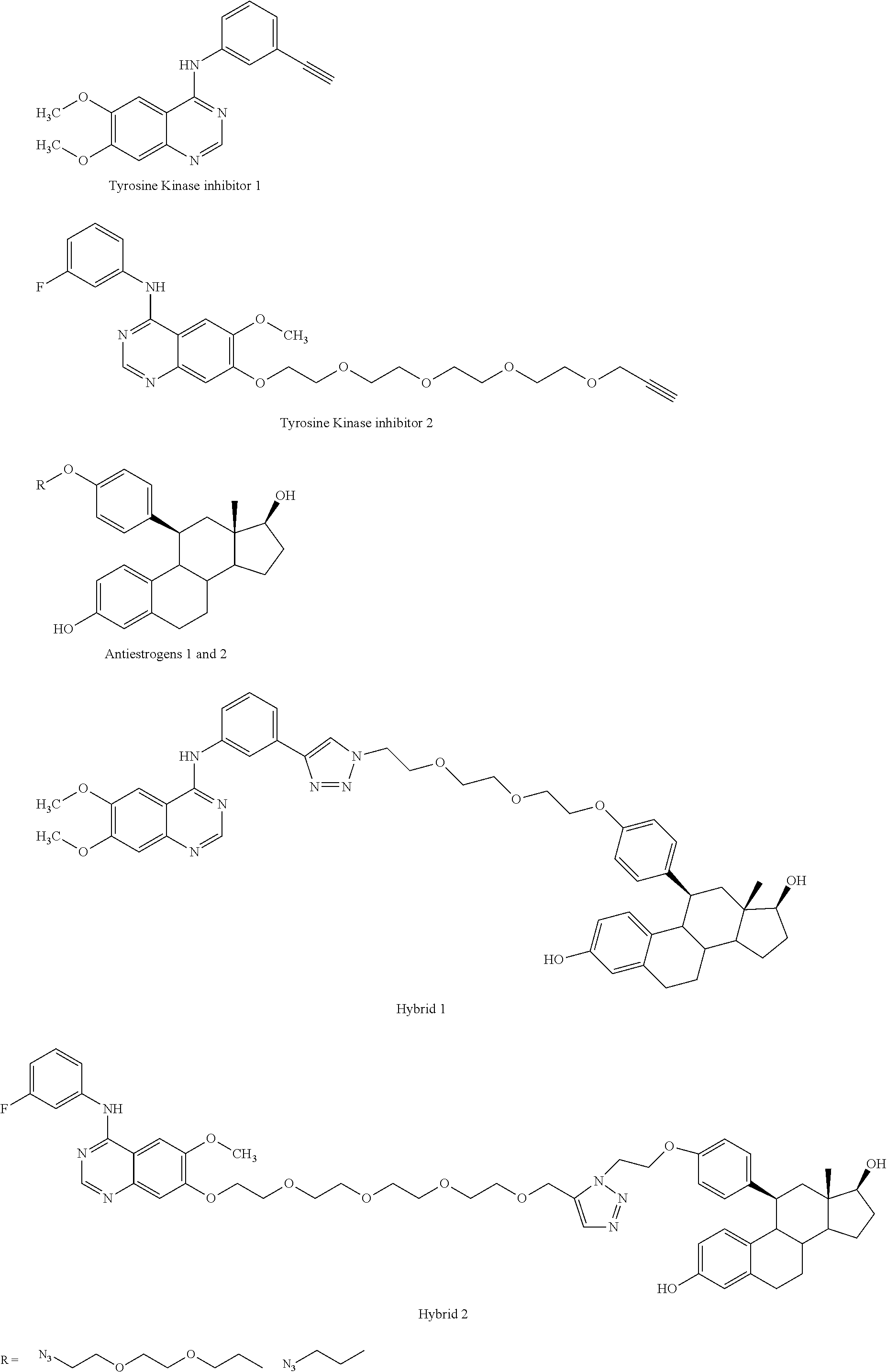

Tyrosine Kinase Receptors and Their Correlation to Breast Cancer

Growth factor receptors or tyrosine kinase receptors (GFP-TKs) are cell surface transmembrane receptors that regulate signal transduction pathways and are divided into 20 subfamilies based on the intracellular kinase domain sequence. One of these families is the epidermal growth factor receptor (EGFR) or ErbB family, consisting of four members: HER-1, HER-2, HER-3 and HERA. Family members contain an extracellular growth factor ligand-binding region, a single membrane spanning region and a cytoplasmic tyrosine-kinase domain (except HER-3). Analogous to ERa, ligand binding to the ErbB family causes dimerization, resulting in the phosphorylation of the target substrate. This phosphorylation activates cellular signaling pathways. Tumor cells can often be “addicted” to tyrosine kinase receptors and inhibition severely impairs tumor growth and survival.

Resistance to adjuvant hormonal therapy in breast cancer is frequently associated with increased EGFR expression. Two members of the EGFR family, HER-1 and HER-2, are overexpressed in 25-30% of breast cancer cases, of which 50% are also ER positive. One of the major pathways regulated by the EGFR family is the MAPK which is known to phosphorylate Serine 118 of ERα's AF-1 domain, the ligand independent region. This phosphorylation results in ERα activation, in the absence of a ligand, leading to binding of ER to estrogen response elements of DNA and initiation of the transcription signaling cascade.

Targeting the EGFR Family and the Pathways it Regulates

There are several approaches to targeting tyrosine kinase receptors (TKRs), more specifically HER-1 and HER-2, and altering the pathways regulated by them. One approach involves the use of monoclonal antibodies (mAbs), which target the extracellular portion of the receptor and result in the clearance of the receptor from the cell surface. Herceptin is an FDA approved mAb for the treatment of metastatic breast cancers that overexpress HER-2. Herceptin promotes cell cycle arrest (typically in the GI phase) and apoptosis by mediating the internalization and subsequent degradation of HER-2, reducing intracellular signaling. Herceptin also reduces the release of angiogenic factors that are controlled by HER-2, such as vascular endothelial growth factor (VEGF), inhibiting tumor angiogenesis.

Another approach is an efficient way to inhibit pathways activated by growth factors. This approach involved developing competitive inhibitors of the adenosine triphosphate (ATP) binding site within the tyrosine kinase catalytic pocket of the receptor, thus blocking the kinase activity and completely abolishes downstream signaling. This effect is accomplished through the use of small molecules known as tyrosine kinase inhibitors (TKI), which can bind to the enzyme either reversibly or irreversibly. Unlike mAbs, TKIs bind competitively to the intracellular kinase domain and therefore must cross the plasma membrane, by passive diffusion. TKIs are able to disrupt a number of processes, most importantly cell proliferation and, like mAbs, prevent tumor angiogenesis by inhibiting the release of angiogenic factors. One of the signaling cascades that TKIs can silence is the MAPK pathway (supra), which activates Era-mediated transcription independent of ligand binding.

A hybrid agent, comprising an anti-estrogen and a TKI, can downregulate both ERα (ligand dependent) and the EGFR signaling (ligand independent) pathways, thereby inhibiting cell transcription via two routes. This hybrid approach provides an opportunity to address drug resistance to hormone therapy. Because both biological targets are intracellular, the estradiol core should facilitate uptake of the TKI across the cell membrane. This hybrid is highly selective since both components are specific for their respective targets. One representative of TKI contains a combination of features present in Gefitinib and Erlotinib, two FDA-approved TKI's of the 4-anilinoquinazoline family that are reversible inhibitors of HER-I.

Salts

The compounds of Formula (I), (Ia), (II), and (III) can also form salts which are also within the scope of this disclosure. Reference to a compound of the present disclosure is understood to include reference to salts thereof, unless otherwise indicated. The compounds of Formula (I), (Ia), (II), and (III) may form pharmaceutically-acceptable (i.e., non-toxic, physiologically acceptable) salts as well as other salts that are also useful, e.g., in isolation or purification steps which can be employed during preparation.

The compounds of Formula (I), (Ia), (II), and (III) which contain a basic moiety, such as, but not limited to, an amine or a pyridine or imidazole ring, can form salts with a variety of organic and inorganic acids. Exemplary acid addition salts include, but are not limited to, acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinases, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of Formula (I), (Ia), (II), and (III) which contain an acidic moiety, such as, but not limited to, a carboxylic acid, can form salts with a variety of organic and inorganic bases. Exemplary basic salts include, but are not limited to, ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups can be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and the like.

Exemplary nonlimiting compounds of Formula (I), (Ia), (II), and (III) are disclosed in the Examples section below. Solvates of the compounds of this disclosure, including hydrates of the compounds, as well as mixtures of the hydrate- and the keto-form of the compounds, are within the scope of this disclosure.

Exemplary Compounds

Exemplary, non-limiting compounds are shown in Table 1.

TABLE 1

| Compound | Structure |
|---|---|
| 1 | 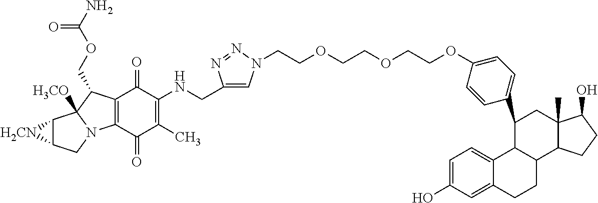 |
| Hybrid 8 | 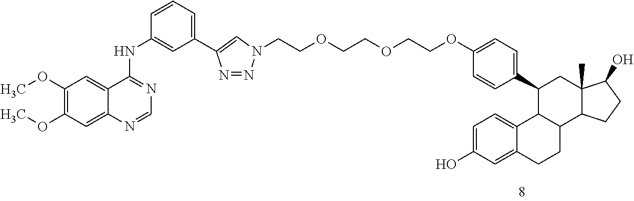 |
| Estradiol | 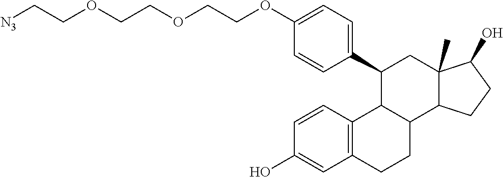 |
| TKI Hybrid 1 | 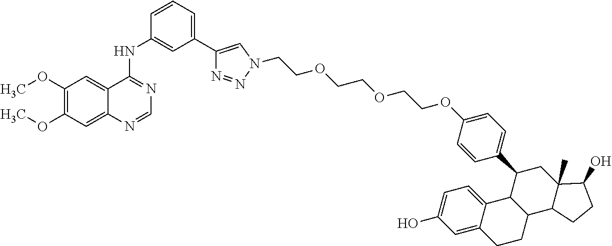 |
| TKI Hybrid 2 | 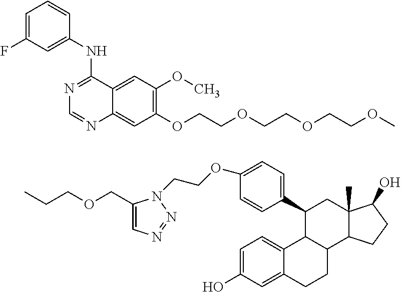 |
| (8S, 9R, 11S, 13S, 14S, 17S)-11-(4-(2- azidoethoxy) phenyl)-7,8,9, 11,12,13,14,15,16, 17-decahydro-13- methyl-6H- cyclopenta[a] phenanthrene- 3,17-diol | 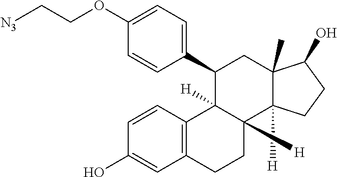 |
| (8S,9R,11S,13S,14S)- 11-(4-(2- azidoethoxy)phenyl)- 7,8,9,11,12,13,15,16- octahydro-3-hydroxy- 13-methyl-6H- | 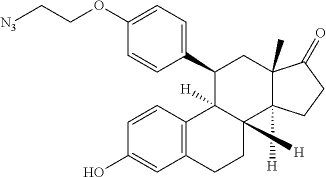 |
| (8S,9R,11S,13S,14S)- 11-(4-(2- azidoethoxy)phenyl)- 7,8,9,11,12,13,14,15, 16,17-decahydro-13- methyl-17-oxo-6H- cyclopenta[a] phenanthren-3-yl acetate | 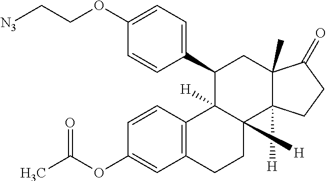 |
| (8S,9R,11S,13S,14S)- 11-(4-(2-(2-(2- azidoethoxy)ethoxy) ethoxy)phenyl)- 7,8,9,11,12,13,15, 16-octahydro-3- hydroxy-13-methyl- 6H-cyclopenta[a] phenanthren- 17(14H)-one | 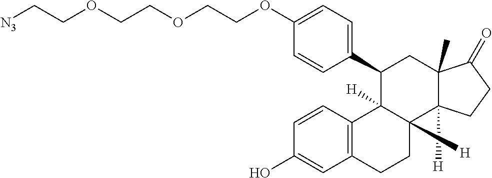 |
| (8S,9R,11S,13S,14S)- 11-(4-(2-(2-(2- azidoethoxy)ethoxy) ethoxy)phenyl)- 7,8,9,11,12,13,14, 15,16,17-decahydro- 13-methyl-17-oxo- 6H-cyclopenta[a] phenanthren-3-yl acetate | 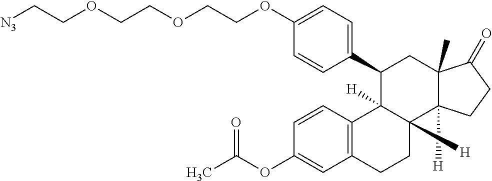 |

### 3. Synthetic Methods

The hybrid compounds according to the disclosure can be made by preparing appropriately substituted terminally functionalized linker derivatives of both the steroidal anti-hormone and the biologically active molecule with the half-linkers. Specific linker chemistry is then used to ligate the two components to form a single molecular entity.

Alternatively, a bifunctional linker that reacts sequentially with each component can also be used to ligate the two components to form a single molecular entity.

An oligoethylene glycol moiety is attached at the 4-position ether linkage. The linker is terminated with functional groups that can be ligated to other biologically active molecules, e.g., antibiotics such as mitomycin C, geldanamycin, dyes for photodynamic therapy, nitroxyl containing molecules for imaging and ROS decomposition, and therapeutic molecules such as receptor tyrosine kinase inhibitors.

The compound shown below incorporates structural features useful in a representative hybrid agent: the 11β-(4-alkoxyaryl) estradiol for anti-estrogenic effects, the alkylamino mitomycin C for DNA binding, and the triethyleneglycol linker to span the two functional groups:

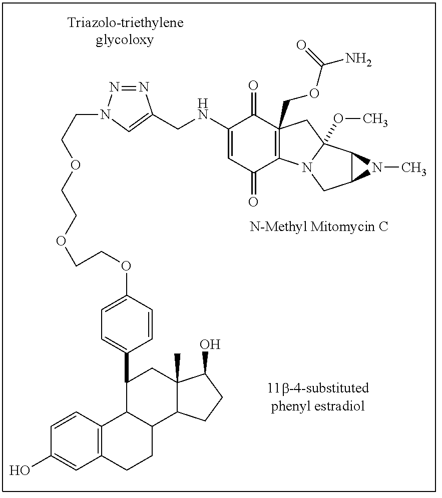

For the preparation of the steroidal component, the Cu(I)-assisted 1,4-addition of aryl Grignard reagents to the steroidal 5,10-α-epoxide was used rather than 1,2-addition to the 1′-oxo steroids, although either method can be used. The incorporation of the protected phenolic group in the aryl moiety subsequently permits the attachment of a variety of substituents via Williamson or Mitsunobu chemistry.

Oligoethylene glycols provide several advantages as linkers. As bifunctional reagents, each terminus can be manipulated. One end can be linked to the phenolic group using either Williamson (via tosylate) or Mitsunobu (via free alcohol) chemistry while the other can be converted to the requisite coupling group (e.g., an azide). The reagents are readily available and possess enhanced hydrophilicity which compensates for the highly non-polar character of the steroidal component.

Mitomycin C was converted to the more stable N-methylated aziridine derivative (porfiromycin), to the 7-methoxy intermediate which undergoes displacement by a variety of amines (e.g., propargyl amine).

The steroid and the mitomycin C, and their analogs, if necessary, can be prepared separately and ultimately ligated using the Huisgen [3+2] cycloaddition reaction. The alkyne and the azido group are both chemically and biologically stable, permitting the evaluation of each unit. Additionally, each group can be coupled to form a small heterocycle that is also chemically and biologically stable.

The biological activity of the individual components can be prepared, characterized, and evaluated to determine the effect of the introduced half-linker onto the specific component prior to ligation. A variety of functionalized biologically active components can be ligated to a single derivatized steroidal anti-hormone, for example, using a common ligation strategy. Alternatively, a terminally modified, biologically active component having a variety of half-linkers can be ligated to a series of complementary steroidal anti-hormone derivatives. Furthermore, by varying or changing the linker length, composition, or ligation chemistry, and by including a greater diversity of biologically active compounds, this methodology can generate substantial diversity. Various illustrative non-limiting examples of ligation strategies that combine a biologically active component and an anti-estrogenic component are shown here:

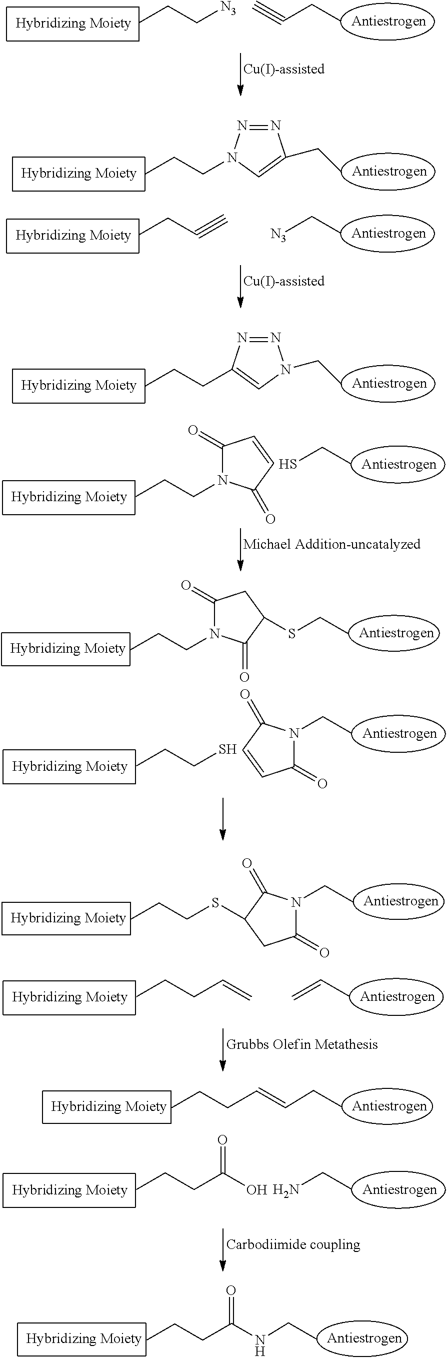

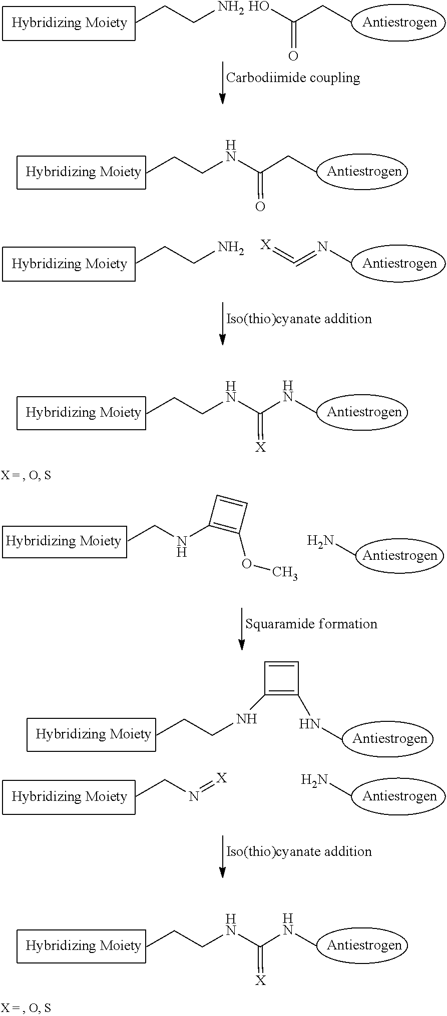

### 4. Methods of Inhibition

This disclosure also provides methods for inhibiting androgen signaling, methods for inhibiting estrogen signaling, methods for inhibiting glucocorticoid signaling, methods for inhibiting the interaction between a co-regulatory protein and an androgen or estrogen receptor, and methods of inhibiting cell proliferation in cancer. The compounds of this disclosure are useful for these methods both in vivo and in vitro.

Heat Shock Protein 90 Disruption

Heat shock proteins (HSP) are molecular chaperones critical for the maintenance of cellular homeostasis through regulation of protein transport, conformational folding and maturation. Heat shock protein 90 (Hsp90) is a 90 kDa protein that is often overexpressed in breast cancer, as well as other cancers, and, as a result of these increased levels, is responsible for maintaining high levels of active oncogenic proteins. One of these proteins is ERα which, when dormant, is confined to the nucleus of the cell, folded in an inhibitory HSP complex. Disruption of Hsp90 leads to improper folding of ERα and its subsequent degradation, resulting in downregulation of its corresponding pathways, such as transcription. Consequently, disruption of Hsp90 provides a promising target for breast cancer therapy.

Geldanamycin (GDA) is an ansamycin benzoquinone antibiotic that binds to the N-terminal ATP binding pocket of Hsp90.

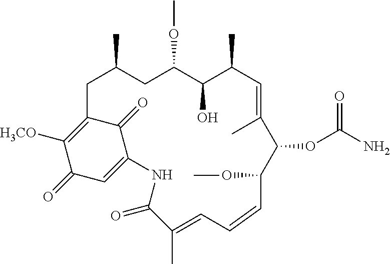

As a result of this binding. Hsp90 is unable to a mediate conformational changes in its client proteins, leading to the degradation of the Hsp90 complex. This imparts antitumor and antiproliferative effects to GDA and the other related ansamycin antibiotics. However, structure activity relationship studies demonstrated that modification at the 17-position of GDA not only generates GDA derivatives that exhibit reduced toxicity, but this position is also substituent tolerant. This hybrid did demonstrate an enhancement of herceptin's antitumor activity by inducing tumor regression in 69% of the recipients compared to 7% of those only receiving herceptin, while maintaining specificity toward HER-2 overexpressing cells. Because GDA's target, Hsp90, is sequestered in the nucleus of the cell in its dormant state, this process requires the release of GDA from the hybrid before degradation in order to achieve maximum effectiveness.

### 5. Methods of Treating

The unique molecular entities provide unique biological properties and can be used to treat various diseases, including cancer, hormone responsive cancer, inflammatory diseases, and hormone-related metabolic disorders. Included in these diseases are Alzheimer's disease, osteoporosis, endometriosis, prostatic hyperplasia, polycystic ovary syndrome. As discussed above, the novel compounds of this disclosure are useful for inhibiting androgen, estrogen, and glucocorticoid signaling. These compounds, or pharmaceutically acceptable salts thereof, are useful for administration in therapeutically effective amounts for inhibiting androgen signaling, estrogen signaling, glucocorticoid signaling, and cell proliferation in a subject.

For example, the compounds of this disclosure can inhibit estrogen signaling and cell proliferation in breast cancer targets. These compounds exhibit affinity for the estrogen receptor binding site and selectivity for that site. Thus, the compounds of this disclosure are useful for treating hormone-responsive breast cancer. The compounds selectively disrupt estrogen signaling mechanisms in breast cancer cells, causing cancer cell stasis or death, while leaving non-cancer, estrogen responsive cells unaffected.

A. Formulation

Treatment of disorders can be accomplished using pharmaceutical formulations comprising at least one compound of Formula (I), (Ia), (II), (III), (IV), or (V), and a pharmaceutically-acceptable carrier which is suitable for administration to a subject.

The compounds of Formula (I), (Ia), (II), (III), (IV), or (V) are administered in a therapeutically effective amount to a patient in need of such treatment. Such an amount is effective in treating a disorder of the patient. This amount can vary, depending on the activity of the agent utilized, the nature of the disorder, and the health of the patient. A skilled practitioner will appreciate that the therapeutically-effective amount of a compound of Formula (I), (Ia), (II), (III), (IV), or (V) can be lowered or increased by fine-tuning and/or by administering more than one compound of (I), (Ia), (II), (III), (IV), or (V), or by administering a compound of Formula (I), (Ia), (II), (III), (IV), or (V) together with a second agent (e.g., antibiotics, antifungals, antivirals, NSAIDS, DMARDS, steroids, etc.). Therapeutically-effective amounts can be easily determined, for example, empirically by starting at relatively low amounts and by step-wise increments with concurrent evaluation of beneficial effect (e.g., reduction in symptoms). The actual effective amount will be established by dose/response assays using methods standard in the art (Johnson et al., Diabetes (1993) 42:1179). As is known to those in the art, the effective amount will depend on bioavailability, bioactivity, and biodegradability of the compound of Formula (I), (Ia), (II), (III), (IV), or (V).

A therapeutically-effective amount is an amount that is capable of reducing a symptom of a disorder in a subject. Accordingly, the amount will vary with the subject being treated. Administration of the compound of Formula (I), (Ia), (II), (III), (IV), or (V) can be hourly, daily, weekly, monthly, yearly, or a single event. For example, the effective amount of the compound can comprise from about 1 μg/kg body weight to about 100 mg/kg body weight. In one embodiment, the effective amount of the compound comprises from about 1 μg/kg body weight to about 50 mg/kg body weight. In a further embodiment, the effective amount of the compound comprises from about 10 μg/kg body weight to about 10 mg/kg body weight. When one or more compounds of Formula (I), (Ia), (II), (III), (IV), or (V) or agents are combined with a carrier, they can be present in an amount of about 1 weight percent to about 99 weight percent, the remainder being composed of the pharmaceutically-acceptable carrier.

Any suitable pharmaceutically acceptable carrier known in the art can be used as long as it does not affect the inhibitory activity of a compound of Formula (I), (Ia), (II), (III), (IV), or (V). Carriers may be used that efficiently solubilize the agents. Carriers include, but are not limited to, a solid, liquid, or a mixture of a solid and a liquid. The carriers can take the form of capsules, tablets, pills, powders, lozenges, suspensions, emulsions, or syrups. The carriers can include substances that act as flavoring agents, lubricants, solubilizers, suspending agents, binders, stabilizers, tablet disintegrating agents, and encapsulating materials. Other examples of suitable physiologically acceptable carriers are described in Remington's Pharmaceutical Sciences (21st ed. 2005), incorporated into this disclosure by reference.

Non-limiting examples of materials which can serve as pharmaceutically-acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline, Ringer's solution, ethyl alcohol; phosphate buffer solutions; and other non-toxic compatible substances employed in pharmaceutical formulations.

The formulations can conveniently be presented in unit dosage form and can be prepared by any methods known in the art of pharmacy. The amount of compound of Formula (I), (Ia), (II), (III), (IV), or (V) which can be combined with a carrier material to produce a single-dosage form will vary depending upon the subject being treated, the particular mode of administration, the particular condition being treated, among others. The amount of active ingredient that can be combined with a carrier material to produce a single-dosage form will generally be that amount of the compound that produces a therapeutic effect. Generally, out of one hundred percent, this amount will range from about 1% to about 99% of active ingredient, in some instances from about 5% to about 70%, in other instances from about 10% to about 30%.

Methods of preparing these formulations or compositions include the step of bringing into association a compound disclosed in this disclosure with a carrier and, optionally, one or more accessory ingredients. In general, the formulations are prepared by uniformly and intimately bringing into association a compound of Formula (I), (Ia), (II), (III), (IV), or (V) with liquid carriers, or timely divided solid carriers, or both, and then, if necessary, shaping the product.

In solid dosage forms of the disclosed compounds for oral administration (e.g., capsules, tablets, pills, dragees, powders, granules, and the like), the active ingredient is mixed with one or more additional ingredients, such as sodium citrate or dicalcium phosphate, and/or any of the following: fillers or extenders, such as, but not limited to, starches, lactose, sucrose, glucose, mannitol, and/or silicic acid; binders, such as, but not limited to, carboxymethyl-cellulose, alginates, gelatin, polyvinyl pyrrolidone, sucrose, and/or acacia; humectants, such as, but not limited to, glycerol; disintegrating agents, such as, but not limited to, agar, calcium carbonate, potato or tapioca starch, alginic acid, certain silicates, and sodium carbonate; solution retarding agents, such as, but not limited to, paraffin; absorption accelerators, such as, but not limited to, quaternary ammonium compounds; wetting agents, such as, but not limited to, cetyl alcohol and glycerol monostearate; absorbents, such as, but not limited to, kaolin and bentonite clay; lubricants, such as, but not limited to, talc, calcium stearate, magnesium stearate, solid polyethylene glycols, sodium lauryl sulfate, and mixtures thereof; and coloring agents. In the case of capsules, tablets, and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols, and the like.

In powders, the carrier is a finely-divided solid, which is mixed with an effective amount of a finely-divided agent. Powders and sprays can contain, in addition to a compound of Formula (I), (Ia), (II), (III), (IV), or (V), excipients, such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Tablets for systemic oral administration can include one or more excipients as known in the art, such as, for example, calcium carbonate, sodium carbonate, sugars (e.g., lactose, sucrose, mannitol, sorbitol), celluloses (e.g., methyl cellulose, sodium carboxymethyl cellulose), gums (e.g., arabic, tragacanth), together with one or more disintegrating agents (e.g., maize, starch, or alginic acid, binding agents, such as, for example, gelatin, collagen, or acacia), lubricating agents (e.g., magnesium stearate, stearic acid, or talc), inert diluents, preservatives, disintegrants (e.g., sodium starch glycolate), surface-active and/or dispersing agent. A tablet can be made by compression or molding, optionally with one or more accessory ingredients.

In solutions, suspensions, emulsions or syrups, an effective amount of a disclosed compound is dissolved or suspended in a carrier, such as sterile water or an organic solvent, such as aqueous propylene glycol. Other compositions can be made by dispersing the agent in an aqueous starch or sodium carboxymethyl cellulose solution or a suitable oil known to the art. The liquid dosage fauns can contain inert diluents commonly used in the art, such as, for example, water or other solvents, solubilizing agents and emulsifiers, such as, but not limited to, ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

Besides inert diluents, the oral compositions can also include adjuvants, such as wetting agents, emulsifying and suspending agents, sweetening, flavoring, coloring, perfuming, and preservative agents.

Suspensions can contain, in addition to the active compound, suspending agents as, for example, ethoxylated isostearyl alcohols, polyoxyethylene sorbitol and sorbitan esters, microcrystalline cellulose, aluminum metahydroxide, bentonite, agar and tragacanth, and mixtures thereof.

Formulations of the pharmaceutical compositions for rectal or vaginal administration can be presented as a suppository, which can be prepared by mixing one or more compounds of this disclosure with one or more suitable non-irritating excipients or carriers comprising, for example, cocoa butter, polyethylene glycol, a suppository wax or a salicylate, and which is solid at RT but liquid at body temperature and, thus, will melt in the rectum or vaginal cavity and release the agents. Formulations suitable for vaginal administration also include, but are not limited to, pessaries, tampons, creams, gels, pastes, foams, or spray formulations containing such carriers as are known in the art to be appropriate.

Dosage forms for the topical or transdermal administration of a compound of this disclosure include, but are not limited to, powders, sprays, ointments, pastes, creams, lotions, gels, solutions, patches, and inhalants. The active compound can be mixed under sterile conditions with a pharmaceutically-acceptable carrier, and with any preservatives, buffers, or propellants.

Ointments, pastes, creams, and gels can contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of the pharmaceutical composition comprising a compound of Formula (I), (Ia), (II), (III), (IV), or (V), to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the compound in a polymer matrix or gel.

B. Administration

Methods of administration of the therapeutic formulations comprising the compounds of Formula (I), (Ia), (II), (III), (IV), or (V) can be by any of a number of methods known in the art, and chosen by a health care clinician. These methods include, but are not limited to, local or systemic administration. Exemplary routes of administration include, but are not limited to, oral, parenteral, transdermal, intradermal, intramuscular, intraperitoneal, intravenous, subcutaneous, intranasal nebulizer, inhaler, aerosol dispenser), colorectal, rectal, intravaginal, and any combinations thereof. In addition, it may be desirable to introduce pharmaceutical compositions of the disclosed compounds into the central nervous system by any suitable route, including intraventricular and intrathecal injection. Intraventricular injection can be facilitated by an intraventricular catheter, for example, attached to a reservoir, such as an Ommaya reservoir. Methods of introduction can be provided by rechargeable or biodegradable devices, e.g., depots. Furthermore, administration can occur by coating a device, implant, stent, or prosthetic. The compounds of Formula (I), (Ia), (II), and (III) can also be used to coat catheters in any situation where catheters are inserted in the body.

The therapeutic formulations containing a compound of Formula (I), (Ia), (II), (III), (IV), or (V) can also be administered as part of a combinatorial therapy with other agents. Combination therapy refers to any form of administration combining two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either simultaneously or sequentially. Thus, an individual who receives such treatment can have a combined (conjoint) effect of different therapeutic compounds.

In the case of cancer, the subject compounds can be administered in combination with one or more anti-angiogenic factors, chemotherapeutics, or as an adjuvant to radiotherapy. It is further envisioned that the administration of the subject compounds will serve as part of a cancer treatment regimen, which may combine many different cancer therapeutic agents.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby.

### Example

### Preparation of the 11β-[4-(\acute{ω}-azido-triethyleneglycoloxy)phenyl]estradiol

A multi-step synthesis provided the key 11β-[4-(ω-azido-triethyleneglycoloxy)]-phenyl-estradiol 8 in good overall yield (Scheme 2).

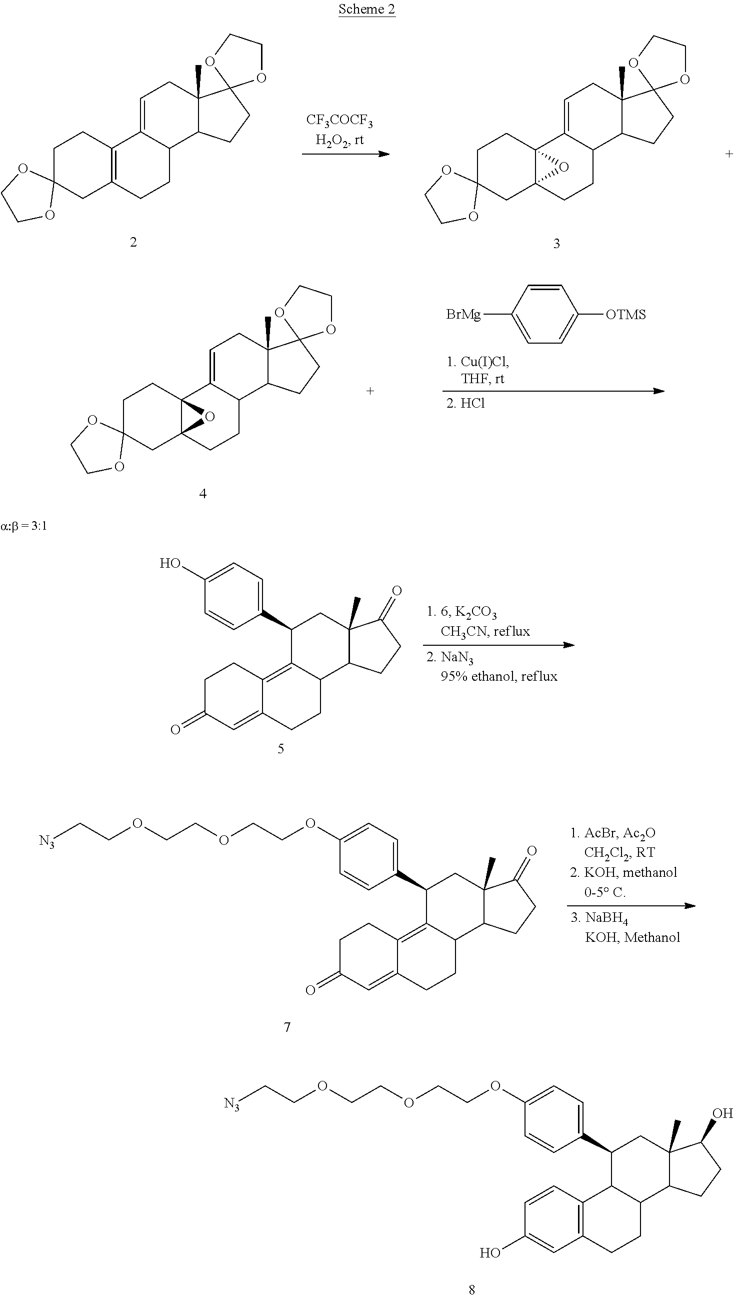

The synthesis of the estradiol component began with the estra-5(10),9(11)-diene 3,17 diethylene ketal as the starting material for the synthesis of the estradiol component. Epoxidation gave selectively the 5,10-α-epoxide (3:1) which was separated from the β-isomer using flash chromatography. Cu(I)-catalyzed 1,4-addition of 4-(trimethylsilyloxy)phenylmagnesium bromide followed by dehydration and deketalization provided the 11β-(4-hydroxyphenyl)-estra-4,9-diene-3,17-dione. Under these conditions, the α-4-hydroxyphenyl steroid is isomerized to the more stable β-isomer. Triethylene glycol was used as the starting material for the half-linker. Tosylation proceeded in high yield to give the ditosylate derivative which underwent Williamson ether synthesis with the 4-hydroxyphenyl steroid. Displacement of the remaining tosylate group with azide followed by aromatization, acetate ester hydrolysis and stereoselective borohydride reduction of the 17-ketone gave the azido-oligoethylene glycoloxyaryl estradiol intermediate.

### Example 2

### Preparation of the Mitomycin C Component

N-propargyl-N′-methyl-mitomycin C 11 was prepared in five steps from mitomycin C as shown in Scheme 3.

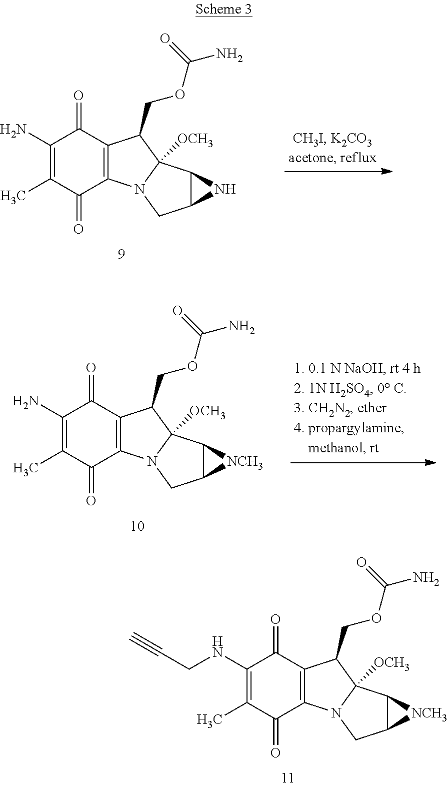

Preparation of the mitomycin C component began with N-methylation of mitomycin C 9 to give porfiromycin 10. ((Sami, et al. J. Med. Chem. (1984) 27: 701-8; lyengar, et al. J. Med. Chem. (1983) 26:16-20) Subsequent hydrolysis of the quinone amine to the hydroxyl intermediate, methylation with diazomethane to give the methyl ether followed by reaction with propargyl amine gave the desired 7-(N-propargyl)-porfiromycin 11. Modifications of the amination step and coalescing the last three steps into a single pot method improved the overall yield.

### Example 3

### Mitomycin C-Estradiol Hybrids

The formation of Hybrid 1 was accomplished by ligating Mitomycin C and an estradiol component according to Scheme 4.

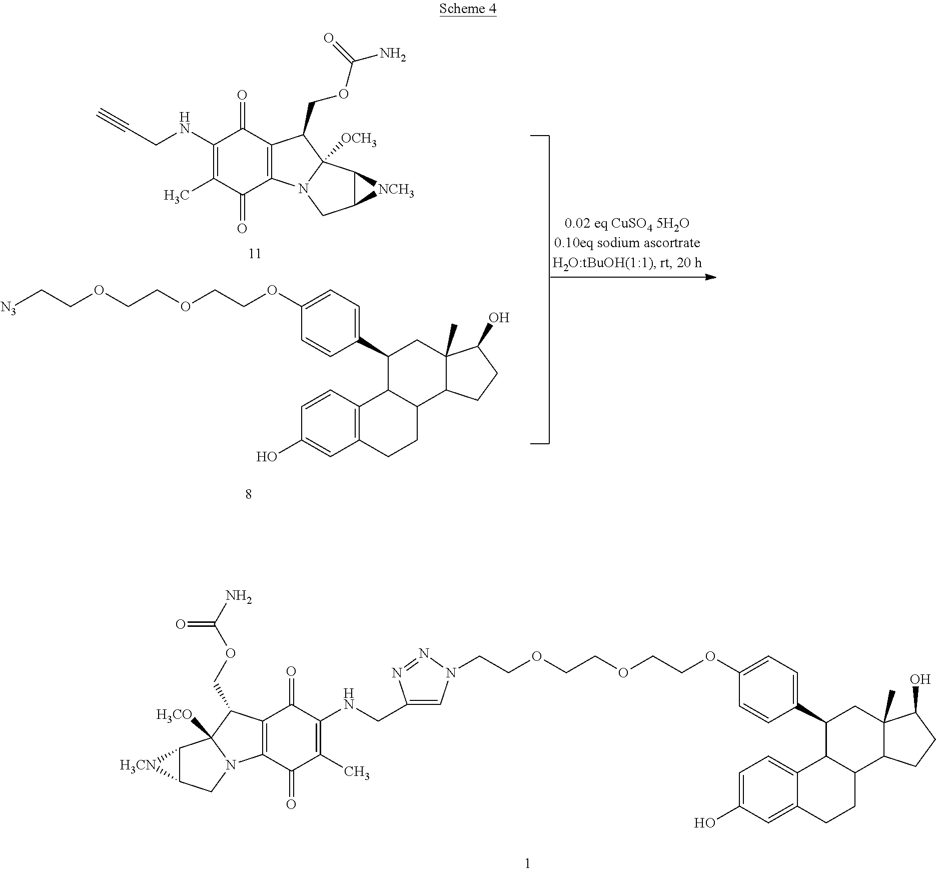

Ligation was accomplished using the [3+2]-cycloaddition reaction between the terminal alkynyl and azido groups. A modification of the conventional method was used, resulting in an isolated yield of 81% for the product 1 which was characterized by ¹H-, ¹³C-NMR and HRMS. Analysis indicated a single [3+2]-cycloaddition product in which the two coupling moieties were trans to one another.

### Example 4

Biological assays evaluating each component demonstrated that both potent anti-estrogenic and anti-proliferative activities were retained in the final hybrid compound, thereby indicating that the linking strategy permitted independent activity within a single molecule.

Assay for Mitomycin C-Estradiol Hybrid as an Anti-Proliferative Agent in Breast Cancer Cells

A. Competitive Binding to Rat Cytosolic ER, Human LBD-ERα and Human LBD-ERβ

Biological evaluation used competitive binding assays with estradiol on the ERα-LBD. Binding affinities of the estradiol derivatives relative to E, were performed in incubations with the LBD of ERα. in lysates of Escherichia coli in which the LBD of human ERα (M₂₅₀-V₅₉₅) is expressed as described in Antonello, et al. (J. Med. Chem. (2006) 49:6642-6645), Morphy, et al. (J. Med. Chem. (2006), 49:4961-4970); Meunier (Accounts Chem. Res. (2007), and Ojima (Accounts Chem. Res. (2007). The assay was perfoiiued overnight in phosphate buffered saline +1 mM EDTA at RT. The competition for binding of [³H]E₂ to the LBD of the E₂-derivatives in comparison to E₂, relative binding affinity (RBA) was determined over a range of concentrations from 10⁻¹² to 10⁻⁶M. After incubation, the media was aspirated, the plates were washed 3 times, and the receptor-bound radioactivity absorbed to the plates was extracted with methanol and counted.

The hybrid compound I competitively displaced estradiol from ERα-LBD with RBA value of 7±1%. The azido-estradiol derivative 8, in the same assay system had an RBA=26±9%, indicating that the presence of the additional mitomycin-C group at the terminus of the linker did not have an adverse effect on ER-LBD binding. These results are shown below in Table 2.

TABLE 2

| | ERα binding | Ishikawa - | Ishikawa - Inhibition |
|---|---|---|---|
| Compound | RBA | RSA | Ki (nM) |
| EH-N9-006 | 0.3 ± 0.2 | 0 | 40 ± 4 |
| EH-N9-019 | 7 ± 1 | 0 | 7 ± 1 |
| EH-N8-040 | 51 ± 13 | 0 | 1 ± 0.05 |
| EH-N7-092 | 26 ± 9 | 0 | 2.4 ± 0.6 |

RBA and RSA are in comparison to E₂ = 100%,
Ki determined in the presence of I nm E₂
EH-N9-006 = n-propargyl mitomycin
EH-N9-019 = mitomycin-estradiol hybrid
EH-N8-040 = napathoquinone-estradiol hybrid
EH-N7-092 = azido-estradiol precursor

B. Estrogenic Potency in Ishikawa Cells

The methodology for determining estrogenic potency, estrogen receptor binding, and stimulation of an estrogen responsive gene in alkaline phosphatase in the Ishikawa cell was performed as described. (Stabile, et al. (2005) Cancer Res. 65:1459-1470; Perez-Soler (2007) Clin. Can. Res. 13(Suppl. 15):4598s-4592s; Nicholson, et al. (2000) British J. Cancer 82:501-513; Schiff, et al. (2004) Clin. Can. Res. 10 (supp.1):331s-336s).

The induction of AlkP in human endometrial adenocarcinoma cells (Ishikawa) grown in 96-well microtiter plates was performed as described. The cells were grown in Phenol red-free medium with estrogen-depleted (charcoal stripped) bovine serum in the presence or absence of varying amounts of the steroids across a dose range of at least 6 orders of magnitude. After 3 days, the cells were washed, frozen, thawed, and then incubated with 5 mM p-nitrophenyl phosphate, a chromogenic substrate for the AlkP enzyme, at pH 9.8. To ensure linear enzymatic analysis, the plates were monitored kinetically for the production of p-nitrophenol at 405 nm.

For antagonists, the effect (Kᵢ) of each compound tested at a range of 10⁻⁶ M to 10⁻¹² M was measured for the inhibition of the action of 10⁻⁹ M E₂ (EC₅₀ about 0.2 nM).

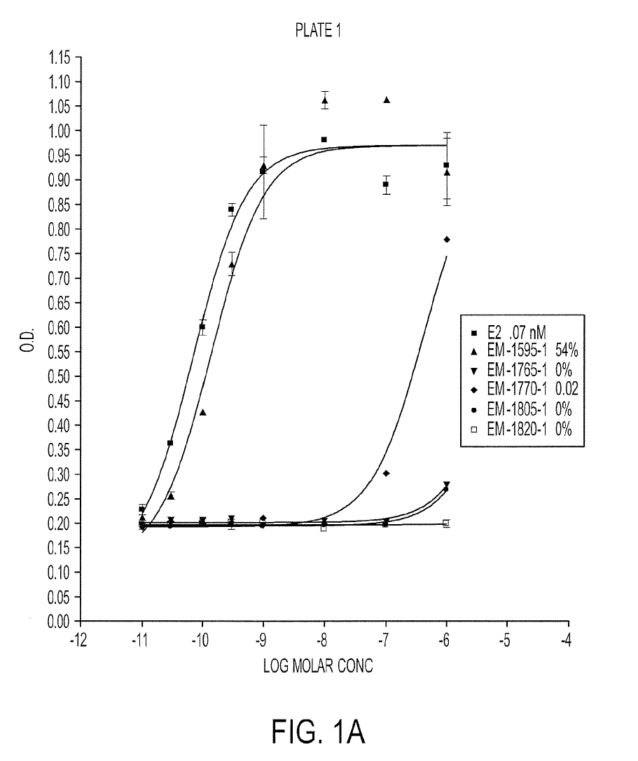

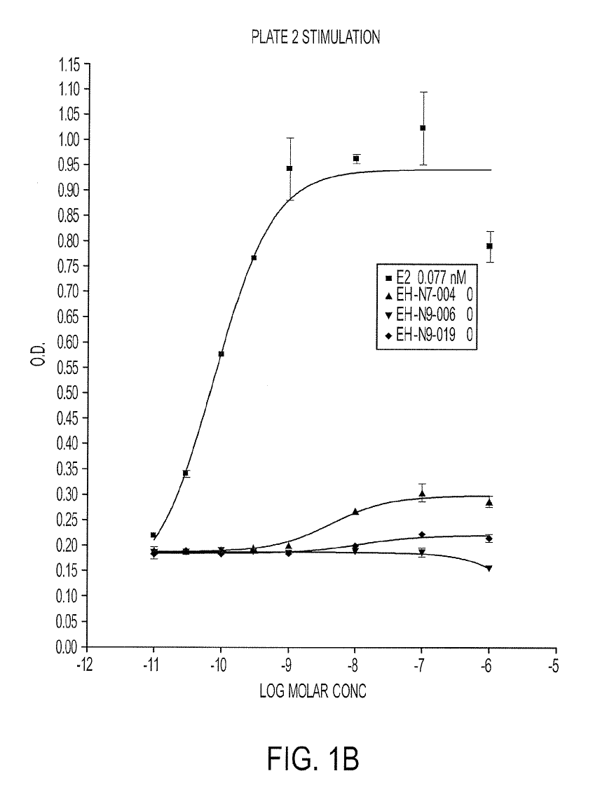

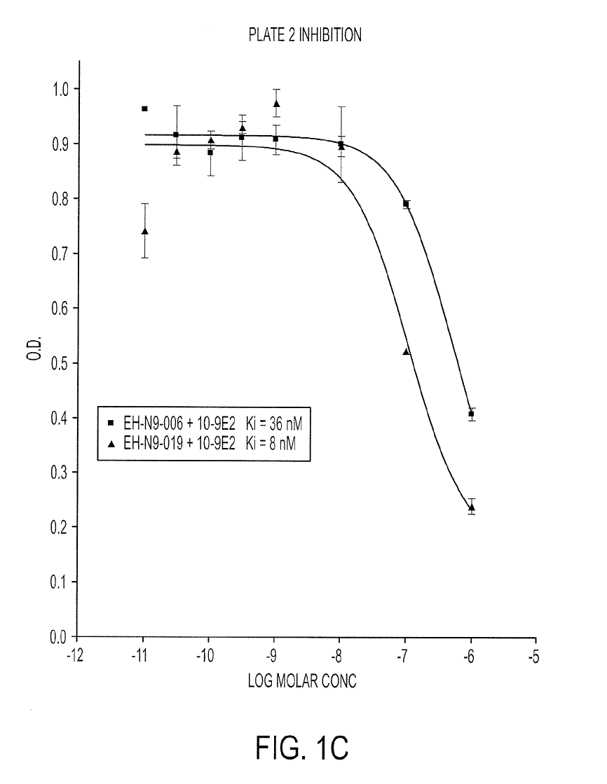

Each compound was analyzed in at least 3 separate experiments performed in duplicate. The Kᵢ and RSA (RSA=ratio of 1/EC₅₀ of the steroid analog to that of E₂×100) were determined using the curve fitting program Prism. The results are shown below in FIGS. 1A-1C, 2, 3, and 4.

The results, as RBAs compared to E₂, of all receptor studies shown in Table 2, are from at least 3 separate experiments performed in duplicate. RBAs represent the ratio of the EC₅₀ of E₂ to that of the steroid analog ×100 using the curve fitting program Prism to determine the EC₅₀.

The hybrid agent did not stimulate the production of alkaline phosphatase, but the compound potently blocked the stimulation caused by 1 nM estradiol. This effect was similar to that shown by the azido-estradiol derivative (Ki=2.4±0.6 nM). These results demonstrated that the presence of the mitomycin C moiety did not interfere with the receptor binding or transcriptional response. The N-propargyl-porifiromycin 11, as expected, exhibited low binding to the ERα-LBD (RBA=0.3±0.2%). The compound exhibited a low level of inhibition in the alkaline phosphatase assay (Ki=40±4 nM), suggesting that at higher doses of the antibiotic analog, that there may be some cytotoxic effects rather than anti-estrogenic action. (Table 2) The data is graphically represented in FIGS. 1B and 1C.

Another series of biological assays (Litzenburger, et al., Clin. Can. Res. (2009) 15:226 and Georghis, et al., Can. Chemothera. Pharmacol. (1992) 29:290.) are run to evaluate the effect of the mitomycin moiety on cellular proliferation of MCF-7 (ER-positive) and MDA-231 (ER-negative) breast cancer cell lines.

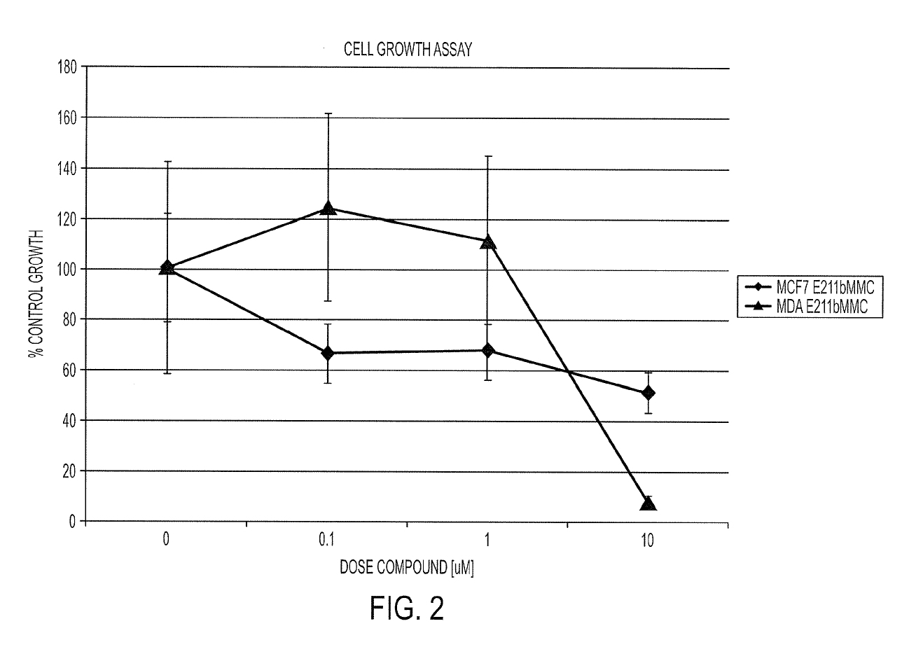

As the results in FIG. 2 demonstrate, mitomycin C is more potent in the MCF-7 vs. the MDA-231 cell lines. Incorporation of the anti-estrogen linked through the triethylene glycol to the mitomycin C has a modest effect compared to mitomycin C on the anti-proliferative response in the MCF-7 cells. In the MDA-231 cells, the response is initially weaker but at high doses the anti-proliferative effect is observed.

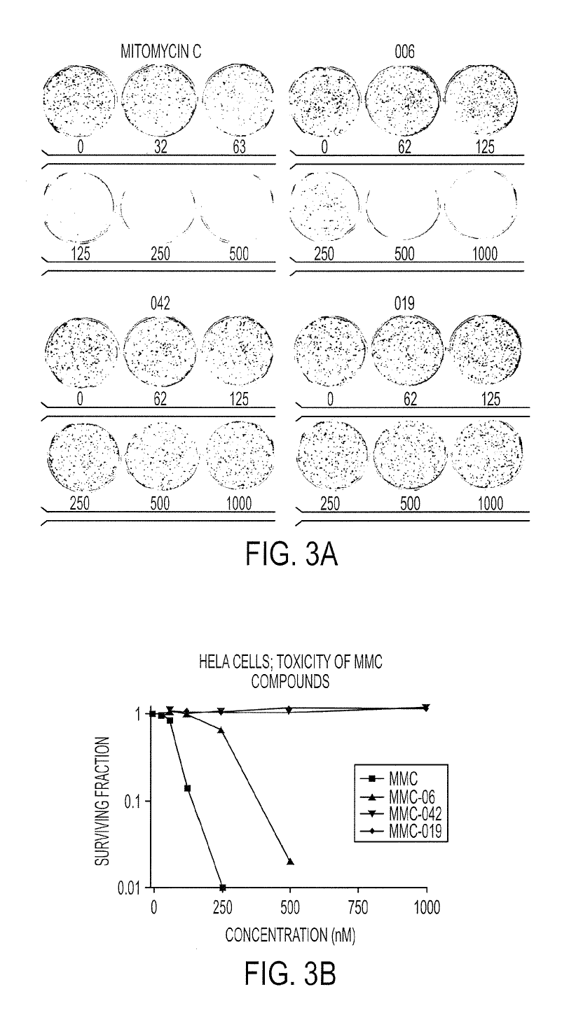

Crystal structures of anti-estrogens bound to the ERα-LBD were used in biochemical studies with MMC. These are shown in FIGS. 3A-3B. The substituent at the 11β-position of estradiol is functionally equivalent to the dialkylaminoalkylphenyl group of the triarylethylene anti-estrogens (hydroxytamoxifen and raloxifene). Therefore, the second oxygen of the linker occupies the same site as the amino group in exerting its effect on helix-12 and specifically on aspartic acid-351. Atoms beyond that point are external to the receptor surface and are accessible for other interactions with either solvent or other proteins. Further interactions with the surface of the estrogen receptor can be significant if those interactions provide complementary binding to the protein. Here, they do not appear to be significant. The addition of substituents beyond the second oxygen of the triethylene glycoloxy group appears to not reduce binding (indicating low steric/electronic demands), nor does it appear to enhance binding (evidence of absence of complementary interactions). Therefore, the triethylene glycoloxy group successfully provided a means for tethering a second molecular component to the steroidal scaffold without compromising ER binding.

Binding studies suggest that interactions of the electrophilic methoxylated carbon and carbamoylated carbon with guanyl residues of DNA (mono or bis alkylation) occur on one face of the Mitomycin C molecule. The amino component associated with the benzoquinone moiety remains solvent accessible and therefore may not provide additional interactions with the DNA.

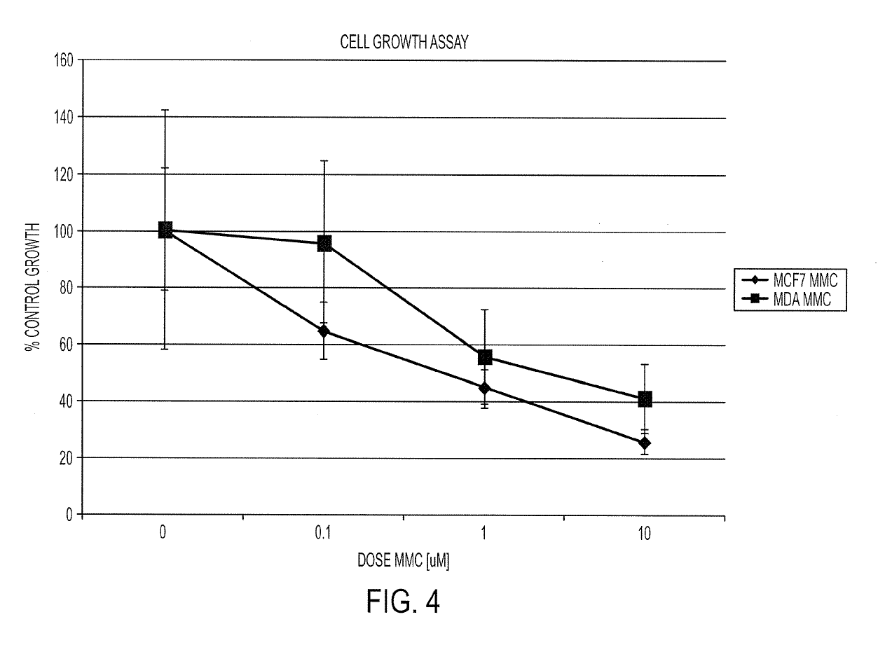

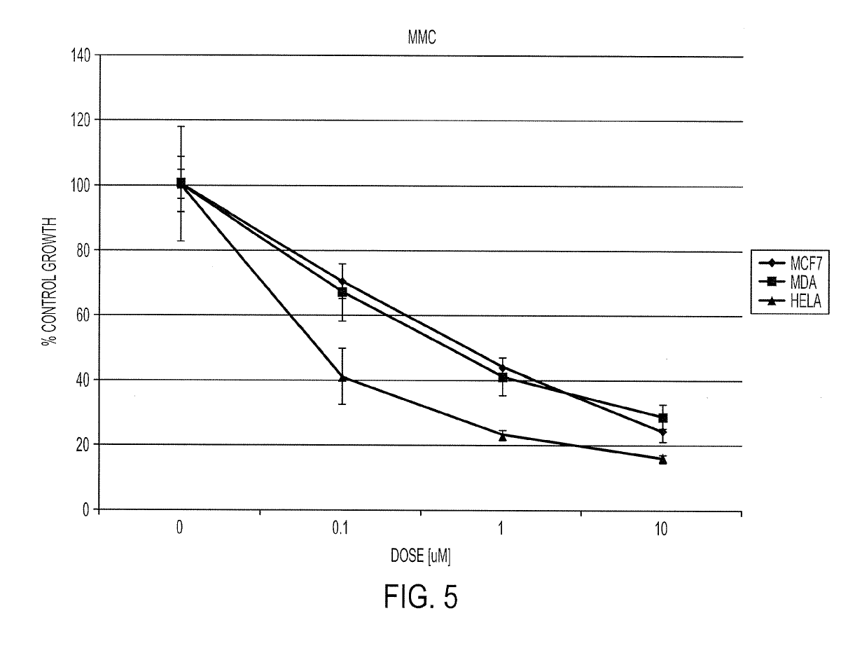

These results, shown in FIGS. 4 and 5, indicate that there are relatively few differences in biological activity among the MMC, N-propargyl, and conjugated derivatives in their ability to induce an anti-proliferative response in the target MCF-7 and MDA-MB-231 cell lines. Therefore, the extended triethylene glyoloxy moiety did not participate in the alkylation events responsible for the anti-proliferative effect. The apparent absence of synergy between the two groups suggests that the individual components do not recruit the complementary protein/DNA targets and, therefore, are not providing simultaneous binding.

In summary, an example of an 11β-substituted anti-estrogen coupled to a DNA-targeted antiproliferative agent was prepared in which the individual biological responses was retained. The antiestrogenic and DNA-targeted effects were not synergistic in these cell lines, the presence of one component did not compromise the biological activity of the other. This hybrid provides a basis for the design and preparation of other estradiol-biomolecule hybrids for modulation of disease processes.

### Example 5

### Synthesis of a 4-(3-Substituted-Anilino)Quinazoline Triethylene Glycoloxy 11β-Phenyl Estradiol Hybrid

Hybrid drug 8 consisting of a 4-anilinoquinazoline tyrosine kinase inhibitor was linked through a triethylene glycol linker to an 11β-substituted estradiol.

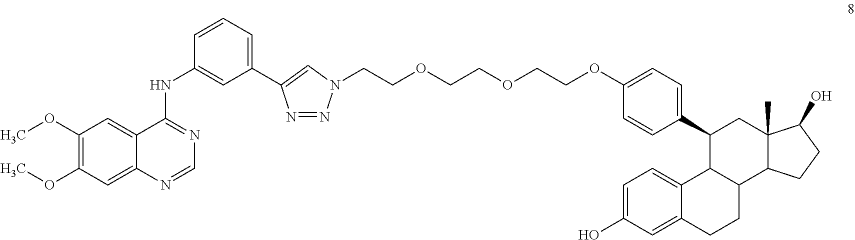

Its synthesis involves the incorporation of an azido-triethylene glycoloxy group at the para-position of the 11β-phenyl estradiol and its ligation to the ethynyl group at the 3-position of the 4-anilino substituent of the tyrosine kinase inhibitor was carried out as shown in Scheme 5.

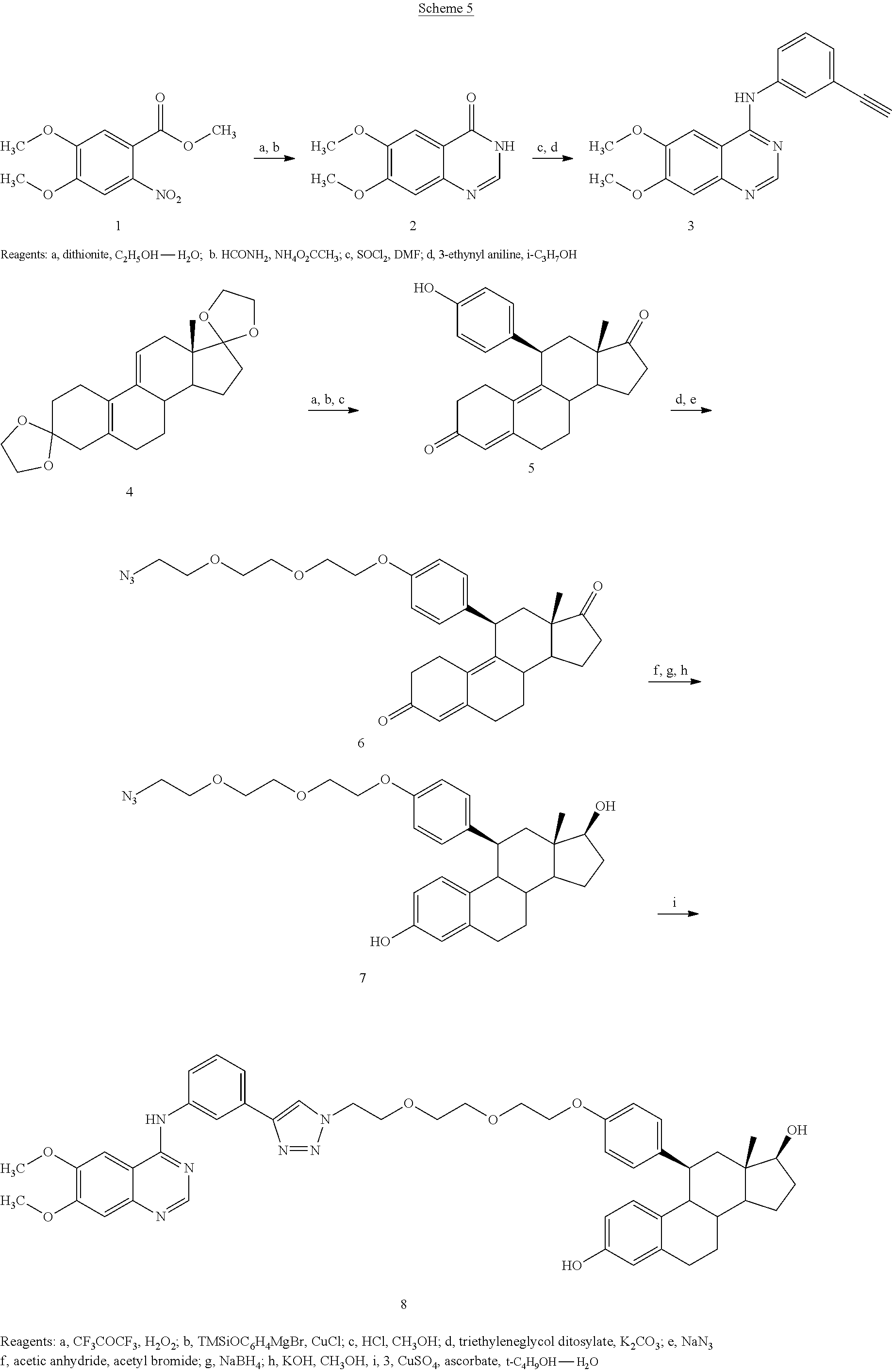

The bis(ethylene glycol ketal) derived from estra-4,9-diene-3,17-dione 4 was utilized as the starting material for the steroidal component. Epoxidation gave the 5,10 oxido isomers (3:1 mixture) in a 76% yield. Without separation, Cu(I)-catalyzed 1,4-addition of the 4-(trimethylsilyloxy)phenyl magnesium bromide gave, after work up, only the 11β-(4-hydroxyphenyl)-estra-4,9-diene-3,17-dione 5 in a 90% yield. Williamson ether formation using the α,ω-bis(tosyl)triethyleneglycol, followed by displacement with azide, gave the 11β-(4-\acute{ω}-azido-triethyleneglycoloxyphenyl)-estra-4,9-diene-3,17-dione 6 (13% yield for the two steps). Aromatization with acetic anhydride-acetyl bromide, followed by reduction and saponification gave the 11β-(4-th-azido-triethyleneglycoloxyphenyl)-estradiol component 7 (56% for three steps). Preparation of the 4-(3-ethynyl-phenyl)amino-6,7-dimethoxy-quinazoline 3 was achieved in four steps from the commercially available ester 1. (Knesl., et al. (2006) Molecules, 11:286-297; Wright, et al. (2001) Bio-org. Med. Chem. Lett., 11:17-21.

Ligation of the steroidal 7 and quinazoline 3 components gave the target hybrid 8 in essentially quantitative yield (99%) using a modification of the standard methods. (Kolb, et al. (2001) Angewandte Chemie. Inter. Ed., 40:2004-2021; Ramachary, et al. (2004) Chem. in Eur. J., 10:5323-5331.)

The study demonstrates that introduction of the 11β-substituent is efficient and the approach tolerates a wide variety of aromatic groups. Introduction of alkynyl groups at other sites on the anilinoquinazoline scaffold can be done, providing entry into other estradiol-TKI hybrids.

### Example 6

### Synthesis of a 4-[(3-Triazolo-Phenyl)Amino]-Quinazoline Triethyleneglycoloxy 11β-Phenyl Estradiol Hybrid

A. Materials and General Methods

All reagents and solvents were purchased from Aldrich or Fisher Scientific. THF and toluene were distilled from sodium/benzophenone. Reactions were monitored by TLC, performed on 0.2 mm silica gel plastic backed sheets containing F-254 indicator. Visualization on TLC was achieved using UV light, iodine vapor and/or phosphomolybdic acid reagent. Column chromatography was performed with 32-63 μM silica gel packing. Melting points were determined using an Electrotherm capillary melting point apparatus and are uncorrected. ¹H NMR spectra were recorded with a Varian Mercury 300 MHz, a Varian 500 MHz or a Bruker 700 MHz spectrometer. DEPT and ¹³C experiments were performed on a Varian Mercury instrument at 75 MHz. NMR spectra chemical shifts are reported in parts per million downfield from TMS and referenced either to TMS, or internal standard for chloroform-d, acetone-d₆, methanol-d₄, and THF-d₈ solvent peak. Coupling constants are reported in hertz. High-resolution mass spectra were obtained by electron impact (EI) or fast atom bombardment (FAB) on MStation JMS700 (JEOL) by University of Massachusetts Amherst, Mass Spectrometry Center using sodium iodide as an internal standard.

B. Syntheses

### 3,17,17-Diethylenedioxy-5,10-α-epoxy-estr-9(11)-ene 3 and

### 3,17,17-Diethylenedioxy-5,10-β-epoxy-estr-9(11)-ene

Estra-5(10),9(11)-diene 3,17 diethylene ketal 4 (1.00 g, 2.79 mmol), hexafluoroacetone trihydrate (0.04 mL, 0.279 mmol), pyridine (0.005 mL), 50% hydrogen peroxide (0.3 mL, 4.74 mmol, ca. 18 M) and dichloromethane (10 mL) were charged into a round bottom flask at RT under argon atmosphere. The mixture was stirred for 20 h at RT (TLC monitoring: ethyl acetate:hexanes, 3:7). After reductive workup (aqueous sodium thiosulfate solution, 2 g in 50 mL of water), the organic layer was washed with water (25 mL×2), extracted with dichloromethane (30 mL×2). The organic layer was dried over magnesium sulfate and concentrated under reduced pressure to give a mixture of isomers (ratio of α:β about 3:1, ¹H NMR). The mixture was purified from other components by chromatographic separation on a silica gel column (25 g, ethyl acetate:hexanes, 1:4). The combined fractions containing the products were concentrated under reduced pressure.

Yield=0.81 g, 76%. R_{f}⁼0.4 (ethyl acetate/hexanes 5:1). ¹H NMR (300 MHz, CDCl₃, δ 6.05 (m, 1H, 3), 5.86 (m, 1H), 0.88 (s, 3H, α), 0.89 (s, 3H, β. ¹³C NMR (75 MHz, CDCl₃) δ 191.1, 164.9, 132.2, 114.5, 55.8.

### 11β-(4-Hydroxyphenyl)estra-4,9-diene-3,17-dione 5

Copper (I) chloride (35 mg, 0.35 mmol) was added at RT to an about 1M solution of 4-(trimethylsilyloxy)phenyl magnesium bromide in THF (10 mL) under argon atmosphere. A solution of the mixture of isomers (ratio about 3:1) (760 mg, 2.03 mmol) in THF (10 mL) was added during about 30 min at RT (exothermic). The mixture was then stirred for 1 h at RT (TLC monitoring: ethyl acetate:hexanes=3:7). When the reaction was complete, the solution was poured into a biphasic mixture of aqueous ammonium chloride (15 equiv, 6 mL) and methylene chloride (8 mL) at 10-15° C. The organic layer was separated, washed with water (20 mL×2), concentrated the total volume to about 5 mL, and diluted with methylene chloride (5 mL). Aqueous hydrochloric acid (6 equiv, 0.47 g in 2.6 mL of water) was added at 0-5° C. This biphasic mixture was stirred for 2 h at 0-5° C. (pH<1, pH paper) and then diluted with water (20 mL). The organic phase was separated, washed with water (20 mL×2) and carefully neutralized to pH of about 8 (10% sodium bicarbonate, about 1.5 mL). The neutralized solution was washed with water (30 mL×2). The organic layer was dried over magnesium sulfate. Compound 5 (0.66 g, 90%) was isolated from a silica gel flash column chromatography (ethyl acetate/hexanes, 3:7).

Yield=0.66 g, 90%. ¹H NMR (300 MHz, CDCl₃): δ 6.75 and 7.07 (AA′BB′, 4H), 5.81 (s, 1H), 4.35 (d, J=7.2 Hz, 1H), 4.01 (t, J=6 Hz, 2H), 2.85 (t, J=6 Hz, 2H), 2.63 (q, J=7 Hz, 4H), 0.56 (s, 3H). ¹³C NMR (75 MHz, CDCl₃): δ 219.3, 200.0, 156.6, 154.3, 145.6, 135.8, 130.2, 128.2, 123.5, 115.9, 50.8, 47.9, 39.8, 38.2, 38.0, 36.9, 35.6, 31.1, 26.9, 26.0, 22.1, 14.6.

### 11β-(4-((2-(2-(2-azidoethoxy)ethoxy)ethoxy)phenyl)-estra-4,9-diene-3,17-dione 6

To a solution of 5 (150 mg, 0.41 mmol) in acetonitrile (20 mL), potassium carbonate (230 mg, 1.64 mmol) was added, and the mixture was heated at about 90° C. for 30 min. The bis α,\acute{ω}-toluenesulfonyl triethylene glycol 6 (380 mg, 0.82 mmol) was charged and stirred at about 90° C. for 18 h (TLC monitoring: ethyl acetate:hexanes, 1:1). The mixture was allowed to cool to RT, and diluted with a mixture of methylene chloride (20 mL) and cold water (about 0° C., 20 mL). After stirring for 30 min, the aqueous layer was extracted with methylene chloride (30 mL×2). The combined organic layers were washed with water (20 mL), brine (20 mL), dried over magnesium sulfate and concentrated to dryness under vacuum. The intermediate (50 mg, 19% yield) was isolated through a silica gel column (50 g) chromatography (ethyl acetate:hexanes, 2:3).

Yield=50 mg, 19%. R_{f}⁼0.4 (ethyl acetate:hexanes=1:1). ¹H NMR (300 MHz, CDCl₃): δ 7.79 and 7.33 (AA′BB′, J=7.8 Hz, J=8.1 Hz, 4H), 7.33 and 6.83 (AA′ BB′, J=7.1, J=8.7 Hz, 4H), 5.79 (s, 1H), 4.38 (d, J=6.6 Hz, 1H), 4.13 (t, J=10.2 Hz, 2H), 4.08 (t, J=4.8 Hz, 2H), 3.82 (t, J=3.3 Hz, 2H), 3.69 (t, J=3.3 Hz, 2H), 3.65 (m, 2H), 3.62 (m, 2H), 0.55 (s, 3H). ¹³C NMR (75 MHz, CDCl₃): S 219.0, 199.4, 157.2, 156.1, 145.1, 145.0, 136.3, 133.3, 130.3, 130.0, 128.2, 128.1, 123.6, 115.0, 71.0, 70.9, 70.0, 69.4, 69.0, 67.6, 57.4, 57.1, 50.9, 47.9, 39.8, 38.2, 37.0, 35.6, 27.0, 26.1, 22.1, 21.8, 14.6.

To a solution of the intermediate (59 mg, 0.1 mmol) in 95% ethanol (5 mL), sodium azide (13 mg, 0.2 mmol) was added. The mixture was stirred at about 90° C. for 18 h, and then evaporated to dryness under reduced pressure. Compound 6 was isolated from silica gel column chromatography (ethyl acetate:hexanes, 2:3).

Yield=35 mg, 67%. ¹H NMR (300 MHz, CDCl₃): δ 7.08 and 6.84 (AA′BB′, J=8.7 Hz, J=9.0 Hz, 4H), 5.80 (s, 1H, C₄—H), 4.38 (d, J=6.6 Hz, 2H, C_{11α}—H), 4.10 (t, J=4.8 Hz, 2H), 3.86 (t, J=5.4 Hz, 2H), 3.74 (m, 2H), 3.68 (m, 2H), 3.39 (t, J=5.1 Hz, 2H), 0.55 (s, 3H, C₁₈—CH₃). ¹³C NMR (75 MHz, CDCl₃): δ 219.0, 199.4, 157.2, 156.1, 145.1, 136.3, 130.3, 128.5, 123.6, 114.9, 71.1, 70.9, 70.3, 67.6, 50.9, 50.9, 47.9, 39.8, 38.2, 38.0, 37.0, 35.6, 31.1, 27.0, 26.1, 22.1, 14.6.

### 11β-(4-((2-(2-(2-azidoethoxy)ethoxy)ethoxy)phenyl)estradiol 7

To a solution of 6 (284 mg, 0.55 mmol) in methylene chloride (20 mL), acetic anhydride (0.05 mL, d=1.080 g/mL, 0.55 mmol) was added slowly under argon atmosphere at RT, followed by acetyl bromide (169 mg, 1.38 mmol). The mixture was stirred at RT for 5 h (TLC monitoring: ethyl acetate:hexanes, 1:1) and then carefully poured into an aqueous solution of sodium bicarbonate (50 mg in 10 mL ice-water). After stirring for 15 h at RT, the mixture was diluted with methylene chloride (50 mL). The organic layer was separated, washed with sodium hydroxide (1N, 25 mL×2), water (25 mL×3, to pH about 7), dried over magnesium sulfate, and concentrated to dryness under reduced vacuum. The crude product (310 mg, 0.55 mmol) was dissolved in methanol (20 mL), and cooled to about 0° C. in an ice-water bath. Potassium hydroxide (62 mg, 1.10 mmol) was added under argon atmosphere. The mixture was stirred at 0° C. for 1.5 h (TLC monitoring: ethyl acetate:hexanes, 1:1). Without further purification, sodium borohydride (50 mg, 1.32 mmol) was added, and stirred for additional 2.5 h at 0° C. (TLC monitoring: ethyl acetate:hexanes, 1:1). After removal of the solvent under reduced pressure, the residue was diluted with methylene chloride (50 mL) and ice-water (50 mL). The aqueous layer was extracted with methylene chloride (25 mL×2). Combined organic layers were washed with water (25 mL), dried over magnesium sulfate, and concentrated to dryness under reduced vacuum. The purification step was performed using column chromatography (silica gel-ethyl acetate:hexanes, 1:1). Compound 8 was collected from combined fractions.

Yield=148 mg, 56% (three-steps). ¹H NMR (300 MHz, CDCl₃): δ 6.63 (d, J=4.8 Hz, 2H), 6.48 (d, J=5.4 Hz, 1H), 6.29 (d, J=5.1 Hz, 2H), 6.25 (d, J=1.5 Hz, 1H), 6.08 (dd, J=5 Hz, J=1.5 Hz, 1H), 4.00 (t, J=2.7 Hz, 2H). 3.92 (t, 1H, H-11α), 3.79 (t, J=3.3 Hz, 1H), 3.70 (m, 3H), 3.65 (m, 4H), 1.77 (t, J=3 Hz, 1H), 2.96 (t, J=9 Hz, 1H), 2.84 (s, 1H), 2.80 (d, J=3 Hz, 1H), 2.49 (dd, 1=1.2 Hz, J=7.7 Hz, 1H), 1.00-2.30 (m, 10H), 0.32 (s, 3H, C₁₈—CH₃) ¹³C NMR (75 MHz, CDCl₃, δ 155.9, 153.3, 137.9, 136.2, 130.8, 127.9, 115.5, 113.8, 113.5, 82.8, 71.0, 70.9, 70.3, 70.1, 67.3, 52.0, 50.9, 47.6, 45.7, 43.8, 38.5, 35.6, 30.7, 30.4, 28.2, 23.4, 13.1. HRMS calcd for C₃₀H₃₉N₃O₅ m/e 521.2890, found m/e 521.2840.

Ligation of Components to Form ER-TKI Hybrid 8

To a reaction flask, the steroidal component (8.2 mg, 0.027 mmole) and the anilinoquinazoline (14 mg, 0.027 mmole) were suspended in a 1:1 mixture of water and t-butyl alcohol (0.8 mL-0.4 mL of water, and 0.4 mL of t-butyl alcohol) at RT. Copper (II) sulfate pentahydrate (0.01 eq, 2.7×10⁻⁴ mmol, 10 uL of freshly prepared solution, 1 mg of copper (II) sulfate in 100 uL of water) was added, followed by sodium ascorbate (0.05 eq, 13.4×10⁻⁴ mmol, 40 uL solution of 0.8 mg of sodium ascorbate in 100 μL of water). The heterogeneous mixture was stirred at RT for 36 h (TLC monitoring: methanol:dichloromethane=1:9). One more equivalent of copper (II) sulfate and of sodium ascorbate were added, followed by stirring for 18 h. When the reaction was complete, ice (aboutlg) was added and thr mixture was stirred for 5 min. The mixture was extracted with dichloromethane (5 mL×3). The organic layer was separated, dried over magnesium sulfate, and concentrated under reduced pressure. The crude product was purified using silica gel column (10 g) chromatography (methanol:dichloromethane=1:9). The combined fractions containing the product were concentrated under reduced pressure to give a white solid of the final product.

Yield=22 mg, 99%. ¹H NMR (300 MHz, CDCl₃): δ 8.23 (s, br, 1H), 8.01 (s, 1H), 7.92 (d, J=7.2 Hz, 1H), 7.71 (s, 1H), 7.36 (t, J=7.8 Hz, 1H), 7.33 (s, 1H), 7.21 (s, 1H), 4.12 (s, 3H, OCH₃), 4.03 (m, 1H), 3.85 (s, 3H, OCH₃), 3.80 (t, J=5.7 Hz, 1H), 3.70 (m, 1H), 3.66 (d, J=4.2 Hz, 1H), 3.11 (s, 1H), 2.8-0.8 (see appendix compound 3-1), 0.06 (s, 3H). ¹³C NMR (75 MHz, CDCl₃): δ 156.7, 155.6, 154.0, 153.4, 150.7, 149.7, 147.4, 146.9, 139.4, 137.8, 136.4, 131.3, 130.7, 130.1, 129.6, 127.7, 121.8, 121.7, 121.6, 119.1, 115.7, 113.6, 109.4, 107.3, 100.3, 82.8, 70.7, 70.0, 69.5, 67.3, 56.5, 56.4, 53.6, 51.9, 51.0, 50.6, 47.4, 45.6, 43.8, 38.4, 35.6, 30.5, 30.3, 28.1, 23.2, 13.1. HRMS calcd. for C₄₈H₅₄N₆O₇ m/e 826.4054, found m/e 826.4026.

### Example 7

### Synthesis of 6- and 7-propargyloxy derivatives of 4-(3-fluoroanilino)-quinazoline

The preparation of the novel isomeric 6- and 7-propargyloxy derivatives of 4-(3-fluoroanilino)-quinazoline was achieved using a 6-step process. An alternate method to the 7-propargyloxy derivative and analogous 7-propargyloxy containing compounds is also described.

The synthesis of the two target compounds was undertaken in parallel (Scheme 6).

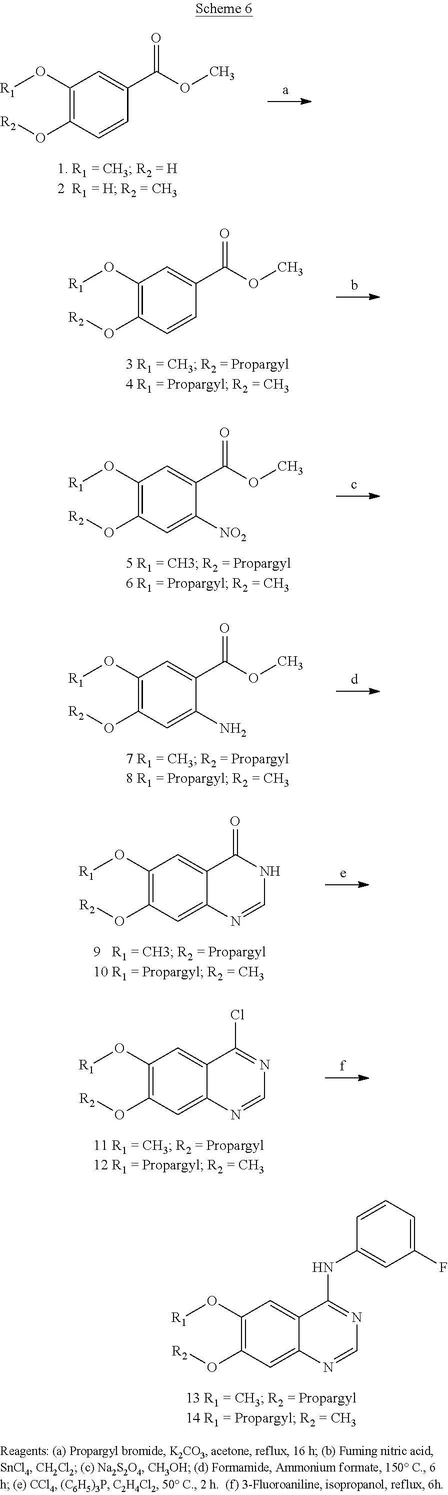

### Scheme 6: Preparation of Isomeric 6- and 7-Propargyloxy-4-(3-Fluoroanilino)quinazolines 13 and 14

Alkylation of the phenolic group of the starting materials 1 and 2 proceeded in essentially quantitative yield. (Hennequin, et al. (2002) J. Med. Chem., 45:1300-1312.) The resultant propargyl ethers 3 and 4 were nitrated using stannic chloride and fuming nitric acid in dichloromethane to give the requisite nitrated intermediates 5 and 6. (DeRuiter, et al. (1986) J. Med. Chem., 29:627-629.) Use of concentrated nitric acid in acetic acid at elevated temperatures, conditions used for nitration of the corresponding benzyl ethers, gave hydration of the propargyl group. Several other reductive methods were evaluated, including stannous chloride (Sydnes, et al. (2007) SynnLett, 1695-1698; Chandregowda, et al. (2007) Org. Proc. Res. and Devel.; Redemann, et al. (1955) Org. Syn., Coll. Vol. 3, 69-70), dithionite (Balcom, D., et al. (1953) Journal of the American Chemical Society, 75, 4334.), Raney nickel-hydrazine (Matsuno, K., et al. (2003) J. Med. Chem., 46, 4910-4925), zinc dust (Wang, et al. (1999) Organic Letters, 1, 1835-1837.) and sodium borohydride-nickel chloride (Gibson, et al. (1997) Bioorganic and Medicinal Chemistry Letters, 7:2723-2728). Although all methods worked well on small scale (1-3 mmole), dithionite and sodium borohydride-nickel chloride were reasonably successful on larger scale (10-20 mmole) reductions to give the anilines 7 and 8. Cyclization of the anilines to the quinazolin-4-ones 9 and 10 was achieved using formamide-ammonium formate in reasonable yields. (Hennequin, et al. (2006) J. Med. Chem., 49:6465-6488; Barlaam, et al. (2005) Bioorg. and Med. Chem. Lett., 15; 5446-5449.)

The identifiable products from that reaction were the depropargylated 4-quinazolines which appeared as fluorescent materials on TLC. Use of carbon trachloride-triphenylphosphine, which proceeds under neutral conditions, gave the requisite 4-chloroquinazoline intermediates 11 and 12 in good yields (Kamal, et al. (2004) Bioorg. and Med. Chem., 12:5427-5366.) Because the quinazolin-4-one intermediates were isolated as partially hydrated materials, additional triphenylphosphine was used to achieve complete consumption of the starting materials. Temperatures greater than 60° C. also produced depropargylated materials in addition to the desired compounds. Subsequent anilination with 3-fluoroaniline in isopropanol at reflux gave the final products 13 and 14 as the hydrochloride salts in 80-90% yields.

Because of the sensitivity of the propargyl group to many of the reagents used in the typical anilino-quinazoline synthesis, the preparation of 13 was also undertaken via an alternate route, proceeding through the 7-O-benzyl intermediate (Scheme 7).

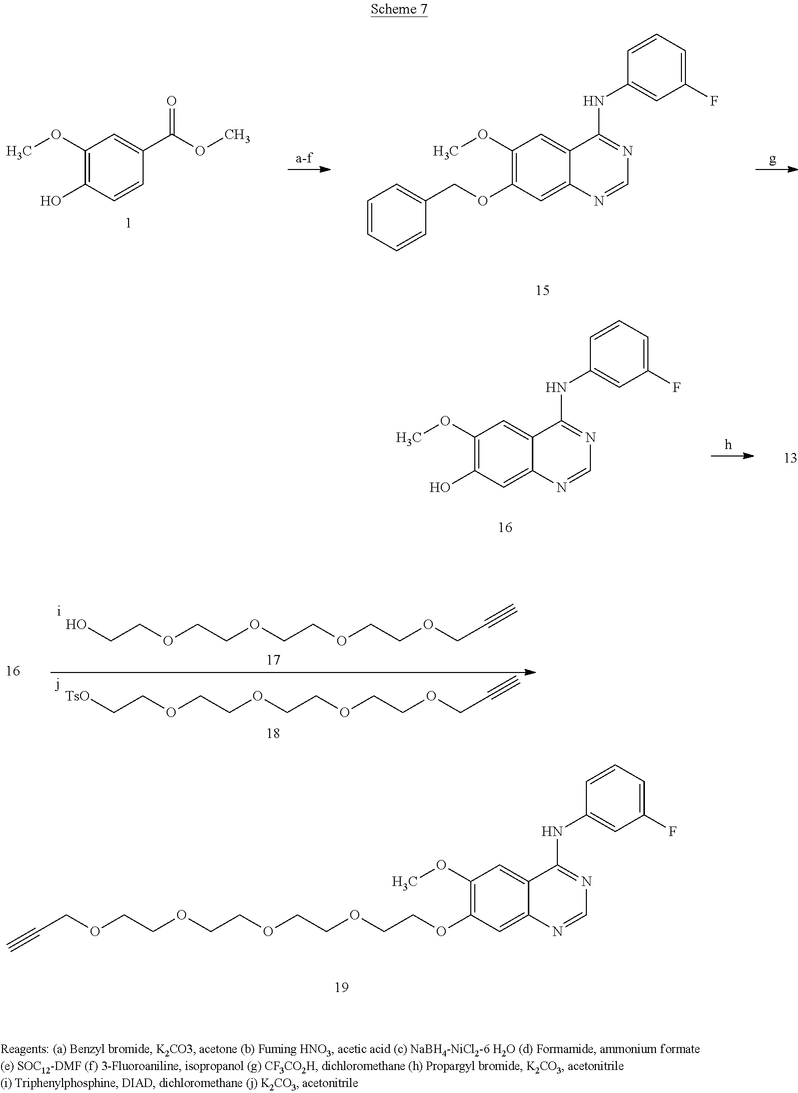

Commercially available methyl 4-hydroxy-3-methoxy-benzoate 1 was converted in 5 steps to 4-(3-fluoroanilino)-6-methoxy-7-benzyloxy-quinazoline 15 (35% overall yield).[17,18] The only significant modification involved an improved workup of the nickel chloride hexahydrate-sodium borohydride reduction of the nitro intermmediate. Debenzylation with trifluoroacetic acid to give the 7-hydroxy intermediate 16, followed by alkylation with propargyl bromide in acetonitrile, gave 13 in a 65% yield for 2 steps. This method involved an additional step, and the overall yield was greater. In addition, the final 7-hydroxy intermediate 16 could be used to generate other products [19]. Introduction of the tetraethylene glycol moiety to give the 7(ω-O-propargyl-tetraethyleneglycoloxy)-analog 19 was achieved both through the Mitsunobu reaction with the mono-alcohol 17 and by alkylation using the mono-tosylate derivative 18 [20]. All the new compounds were readily purified by crystallization or chromatography, and characterized by ¹H-NMR and elemental analysis/mass spectrometry.

The propargyloxy derivatives 13, 14 and 19 serve as coupling partners, via Huisgen [3+2] cycloaddition, for our azido-substituted derivatives of other biomolecules of interest, such as anti-estrogens, fluorophores and radiolabels.

### Example 8

### Additional Hybrid Syntheses

Materials and Methods

All reagents and solvents were purchased from Aldrich or Fisher Scientific. THF and toluene were distilled from sodium/benzophenone. Reactions were monitored by TLC, performed on 0.2 mm silica gel plastic backed sheets containing F-254 indicator. Visualization on TLC was achieved using UV light, iodine vapor and/or phosphomolybdic acid reagent. Column chromatography was performed with 32-63 μm silica gel packing. Melting points were determined using an Electrotherm capillary melting point apparatus and are uncorrected. ¹H NMR spectra were recorded with a Varian Mercury 300 MHz, a Varian 500 MHz or a Bruker 700 MHz spectrometer. NMR spectra chemical shifts are reported in parts per million downfield from TMS and referenced either to TMS, or internal standard for chloroform-d, acetone-d₆, methanol-d₄, and THF-d₈ solvent peak. Coupling constants are reported in hertz. High-resolution mass spectra were obtained by electron impact (EI) or fast atom bombardment (FAB) on MStation JMS700 (JEOL) by University of Massachusetts Amherst, Mass Spectrometry Center using sodium iodide as an internal standard. Elemental analyses were performed by Columbia Analytical Services, Kelso, Wash.

### Synthesis of Methyl 3-methoxy-4-(prop-2-ynyloxy)benzoate 3

Methyl 3-methoxy-4-hydroxybenzoate 1 (18.22 g, 0.100 mol) was dissolved in acetone (200 mL) and potassium carbonate (48.8 g, 0.400 mol) was added. Propargyl bromide (80% 9N toluene, 26 mL, 0.30 mol) was added, and the mixture was heated at reflux with stirring for 7 h. The mixture was filtered while hot, the filtrate was evaporated to dryness, and the residue was recrystallized from methanol.

Yield=17.25 g, 0.0784 mol, 78%, white needles, mp 87-88° C. ¹H-NMR: 7.672 (dd, 1H, C₆—H, J=8.5, 2.0 Hz); 7.568 (d, 1H, C₂—H, J=2 Hz); 7.044 (d, 1H, C₅—H, J=8.5); 4.825 (d, 2H, J=2.5 Hz); 3.927 (s, 3H); 3.897 (s, 3H); 2.54 (t, 1H, J=2.5 Hz). C₁₂H₁₂O₄: Calc. C=65.45; H=5.59. Obs. C=65.34; H=5.65.

### Synthesis of Methyl 4-methoxy-3-(prop-2-ynyloxy)benzoate 4

Methyl 3-hydroxy-4-methoxybenzoate 2 (12.4 g, 0.0681 mol) was treated in the same manner as described for compound 3.

The colorless needles weighed 13.44 g, 0.061 mol, 90%. Mp 84-85° C. ¹H-NMR: 7.733 (dd, 1H, C₆—H, J=8.5, 2.5 Hz); 7.690 (d, 1H, C₂—H, J=2.0 Hz); 6.914 (d, 1H, C₅—H, J=8.5 Hz); 4.808 (d, 2H, J=2.5 Hz); 3.931 (s, 3H); 3.896 (s, 3H); 2.530 (t, 1H, J=2.5 Hz). C₁₂H₁₂O₄: Calc. C=65.45; H=5.59. Obs. C=65.28; H=5.55.

### Synthesis of Methyl 5-methoxy-2-nitro-4-(prop-2-ynyloxy)benzoate 5

3 (7.85 g, 0.0357 mol) was dissolved in dichloromethane (250 mL) and chilled with an ethylene glycol-dry ice slush. SnCl₄ (in dichloromethane, 1M, 53 mL, 0.053 mol) and fuming nitric acid (6 mL, about 0.05 mol) were added slowly to the rapidly stirred cold solution. After 10 min the solution was allowed to warm to RT and was stirred for 45 min. The mixture was poured into water, partitioned, and the organic phase was washed twice with brine, dried over magnesium sulfate, filtered, the solvents removed by evaporation, and the residue was recrystallized from methanol.

The pale yellow needles weighed 8.38 g, 0.032 mol, 89%. Mp 111-112° C. ¹H-NMR: 7.750 (s, 1H, C2-H); 7.198 (s, 1H, C₅—H); 4.956 (d, 2H, J=2.4 Hz); 4.081 (s, 3H); 4.024 (s, 3H); 2.702 (t, 1H, J=2.4 Hz). C₁₂H₁₁NO₆: Calc. C, 54.34; H=4.18. Obs. C=54.29; H=4.30.

### Synthesis of Methyl 4-methoxy-2-nitro-5-(prop-2-ynyloxy)benzoate 6

4 (3.30 g, 0.015 mol) was treated in the same manner as described for compound 3.

The resulting pale yellow needles weighed 2.73 g, 0.0103 mol, 69%. Mp 125-126° C. ¹H-NMR: 7.443 (s, 1H, C₂—H); 7.260 (s, 1H, C₅—H); 4.856 (d, 2H, J=2.5 Hz); 3.970 (s, 3H); 3.909 (s, 1H); 2.600 (t, 1H, J=2.5 Hz). C₁₂H₁₁NO₆: Calc. C=54.34; H=4.18. Obs. C=54.19; H=4.28.

### Synthesis of Methyl 2-amino-5-methoxy-4-(prop-2-ynyloxy)benzoate 7

5 (1.00 g, 0.0038 mol) was dissolved in hot methanol (125 mL). Sodium dithionite (4.00 g, 0.023 mol) was added, followed by 10 mL water. The slurry was maintained at 55-65° C. for 1 h or until the reaction was complete (TLC). The reaction mixture was then concentrated and partitioned between ethyl acetate and brine. The organic phase was dried over magnesium sulfate, filtered and the solvent evaporated. The residue was recrystallized from methanol.

Yield=0.804 g, 0.0034 mol, 90%. Mp 118-119° C. ¹H-NMR: 7.315 (s, 1H, C₂—H); 6.303 (s, 1H, C₅—H); 4.760 (d, 2H); 3.854 (s, 3H); 3.821 (s, 3H); 2.545, (t, 1H). C₁₂H₁₃NO₄: Calc. C=61.27; H=5.57. Obs. C=61.32; H=5.60.

### Synthesis of Methyl 2-amino-4-methoxy-5-(prop-2-ynyloxy)benzoate 8

6 (1.00 g, 0.0038 mol) was treated in the same manner as was 5, with the exception that twice the reaction time (2 h) was used. The residue was recrystallized from methanol.

Yield=0.46 g, 0.0020 mol, 53%. Mp 98.0-100.0° C., ¹H-NMR: 7.500 (s, 1H, C₂—H); 6.151 (s, 1H, C₅—H); 5.4 (brd s, 2H, NH₂); 4.573 (d, 2H); 3.857 (s, 6H); 2.518 (t, 1H). C₁₂H₁₃NO₄: Calc. C=61.27; H=5.57. Obs. C=61.49; H=5.59.

### Synthesis of 6-Methoxy-7-(prop-2-ynyloxy)quinazolin-4(3H)-one 9

7 (0.86 g, 0.0036 mol) was added to 10 mL of formamide, and ammonium formate (0.35 g, 0.0055 mol), and the solution was heated at reflux for 5 h. The reaction mixture was then poured into water and extracted 4× with ethyl acetate. The combined organic phases were washed twice with brine, dried over sodium sulfate, filtered and evaporated to dryness. The residue was recrystallized from methanol-ethyl acetate.

Yield=0.331 g, 0.00144 mol, 43%. Mp 161-162° C., pale amber needles. ¹H-NMR (d₆-DMSO): 12.103 (br s, 1H); 7.990 (d, 1H, C₂—H, J=3.5 Hz); 7.473 (s, 1H, C₅—H); 7.230 (s, 1H, C₅—H); 4.983 (d, 2H, J=2.5 Hz); 3.881 (s, 3H); 3.651 (t, 1 H, J=2.5 Hz). C₁₂H₁₀N₂O₃ 0.5 H₂O:Calc. C=62.60, H=4.38. Obs. C=60.57; H=4.88.

### Synthesis of 7-Methoxy-6-(prop-2-ynyloxy)quinazolin-4(3H)-one 10

The aniline 8 (0.970 g, 0.00413 mol) was dissolved in 10 mL formamide, and ammonium formate (0.50 g, 0.0080 mol) was added, and the solution was heated at 150° C. for 16 h. The reaction mixture was then treated as described for compound 9.

Mp>260° C., pale amber solid, 0.687 g, 0.0030 mol, 72%. ¹H-NMR (d6-DMSO): 12.14 (br s, 1H, N—H); 8.00 (s, 1h, C₂—H); 7.58 (s, 1 h, C₅—H); 7.16 (s, 1H, C₈—H); 4.92 (d, 2H, J=2.5 Hz, CH₂—O—); 3.91 (s, 3H, CH₃—O—); 3.62 (t, 1H; J=2.5 Hz, HC—) C₁₂H₁₀N₂O₃: Calc. C=62.60, H=4.38. Obs. C=62.16; H=4.57.

### Synthesis of 4-Chloro-6-Methoxy-7-(prop-2-ynyloxy)quinazoline 11

To a suspension of 9 (0.212, 0.9 mmol) in 1,2-dichloroethane (10 mL) were added carbon tetrachloride (0.8 mL) and triphenylphosphine (0.52 g, 2.0 mmol). The reaction mixture was warmed to 80° C. and the temperature maintained until all starting material had been consumed. The solution was cooled, evaporated to dryness and the residue was purified by column chromatography on silica gel (hexane-ethyl acetate, 7:3). Depropargylated 4-chloro-6-methoxy-7-hydroxy-quinazoline (0.060 g, 0.028 mmol, 30%) was eluted first, followed by 11 (0.135 g, 0.54 mmol, 60%). The product was recrystallized from iso-propanol to give colorless needles.

Mp 207-209° C., ¹H-NMR (CDCl₃): 8.890 (s, 1H, C₂—H); 7.520 (s, 1H, C₅—H); 7.444 (s, 1H, C₈—H); 4.956 (d, 2H, J=2.5 Hz); 4.086 (s, 3H), 2.595 (t, 1H, J=2.5 Hz).

### Synthesis of 4-Chloro-7-Methoxy-6-(prop-2-ynyloxy)quinazoline 12

Compound 12 was obtained using the same procedure as for 11. Depropargylated 4-chloro-6-hydroxy-7-methoxy quinazoline (0.032 g, 0.15 mmol, 25%) was eluted first, followed by 12 (0.059 g, 0.22 mmol, 35%). The product was recrystallized from iso-propanol to give colorless needles.

Mp 196-198° C., ¹H-NMR (CDCl₃): 8.920 (s, 1H, C₂—H); 8.622 (s, 1H, C₅—H); 7.344 (s, 1H, C₈—H); 4.963 (d, 2H, J=2.5 Hz); 4.054 (s, 3H); 2.604 (t, 1H, J=2.5 Hz).

### Synthesis of 4-[(3-Fluorophenyl)amino]-6-methoxy-7-(prop-2-ynyloxy)quinazoline Hydrochloride 13

Method A

To isopropanol (3 mL) were added 11 (0.050 g, 0.20 mmol) and 3-fluoroaniline (0.22 g, 2 mmol) and the resultant solution was heated at reflux for 3 h. The reaction mixture was cooled and the resultant precipitate was collected by filtration, washed with cold isopropanol and dried, yielding 13 (0.062 g, 0.17 mmol, 85%).

Method B

To a mixture of 16 (0.125 g, 0.40 mmol) and potassium carbonate (0.12 g, 0.9 mmol) in 2.0 mL DMF was added propargyl bromide in toluene (0.10 mL). The reaction mixture was heated at 50-60° C. for 16 h. The mixture was cooled to ambient temperature and poured in to water. The solution was extracted with ethyl acetate; the organic layer was washed with brine, dried over magnesium sulfate, filtered and evaporated to dryness.

Crystallization from iso-propanol gave the product as the free base (0.072 g, 0.22 mmol, 55%). Mp (HCL salt) 259-262° C., ¹H-NMR free base (CDCl₃): 8.694 (s, 1H, C₂—H); 7.738 (dt, 1H, C_{2′}—H); 7.421 (s, 1H, C₅—H); 7.360-7.255 (m, 2H, C_{6′}— and C_{4′}—H); 7.101 (s, 1H, C₈—H); 6.827 (td, 1H, C_{5′}—H); 4.912 (d, 2H, J=2.5 Hz); 4.035 (s, 3H,; 2.582 (t, 1H, J=2.5 Hz).

### Synthesis of 4-[(3-Fluorophenyl)amino]-7-methoxy-6-(prop-2-ynyloxy)quinazoline Hydrochloride 14

To isopropanol (1.5 mL) were added 12 (0.034 g, 0.14 mmol) and 3-fluoroaniline (0.15 g, 1.4 mmol) and the resultant solution was heated at reflux for 3 h. The reaction mixture was cooled and the resultant precipitate was collected by filtration, washed with cold isopropanol and dried.

Yield=14 (0.032 g, 0.088 mmol, 63%) Mp 252-256° C., ¹H-NMR. (CD₃COCD₃): 9.013 (bs, 1H, N—H), 8.588 (s, 1H, C₂—H), 8.000 (dt, 1H, C_{2′}—H), 7.805 (s, 1H, C₅—H), 7.561 (d, 1H, C_{6′}—H), 7.370 (q, 1H, C_{4′}—H), 7.247 (s, 1H, C₈—H), 6.838 (td, 1H, C_{5′}—H), 4.920 (d, 2H, J=2.5 Hz), 4.002 (s, 3H), 3.151 (t, 1H, J=2.5 Hz).

### Synthesis of 4-[(3-Fluorophenyl)amino-6-Methoxy-7-(3,6,9,12-tetra-oxa-pentadecan-14-ynyloxy)-quinazoline 19

Method A

To a solution of 16 (0.40 g, 1.4 mmol), 17 (0.40 g, 1.7 mmol) and triphenylphospine (0.45 g, 1.7 mmol) in 30 mL dichloromethane was added slowly, with stirring, a solution of diisopropyl azodicarboxylate (0.35 g, 1.7 mmol) in 10 mL dichloromethane. The resultant solution was stirred at ambient temperature for 16 h, evaporated to dryness, and the resultant residue was separated by column chromatography on silica gel using a step gradient from 1% to 5% methanol in dichloromethane. The product was isolated as a colorless oil (0.27 g, 0.54 mmol, 38%).

Method B

To acetonitrile (5 mL) were added 16 (0.070 g, 0.24 mmol), O-propargyhtetraethyene glycol tosylate 18 (0.125 g, 0.32 mmol), and potassium carbonate (0.140 g, 1.0 mmol). The reaction mixture was heated at 60° C. with stirring for 24 h, cooled to ambient temperature, filtered and the filtrate was evaporated to dryness. The residue was purified by column chromatography on silica gel using methanol-dichloromethane (2:98) as the eluent.

The product was isolated as a colorless oil (0.75 g, 0.15 mmol, 60%). Method B. ¹H-NMR (CD₃COCD₃): 8.90 (bs, 1H, NH); 8.58 (s, 1H, C₂—H); 8.00 (dt, 1H, C_{2′}—H); 7.68 (s, 1H, C₅—H); 7.57 (d, 1H, C_{6′}—H); 7.34 (q, 1H, C_{4′}—H); 7.20 (s, 1H, C₈—H); 6.83 (t, 1H, C_{5′}—H); 4.31 (t, 2H); 4.16 (d, 2H, J=2.5 Hz); 3.92 (s, 3H); 3.70-3.50 (m, 14H), 2.92 (t. 1H, J=2.5 Hz).

### Example 9

### Synthesis of 11β-N-methyl-propargyl Amine-(2-phenoxyethyl)3-acetoxy-17-oxo-estra-1,3,5(10)-triene

The Mitsunobu reaction, shown below, was performed using standard conditions, and afforded 11β-N-methyl-propargyl amine-(2-phenoxyethyl)-3-acetoxy-17-oxo-estra-1,3,5(1O)-triene 2-19.

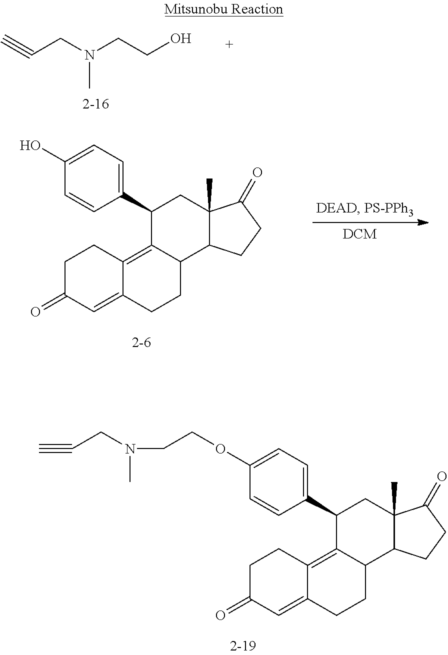

2-19 then underwent the aromatization, saponification and reduction reactions as azide 2-13 to provide the complimentary alkynyl anti estrogen, 2-21.

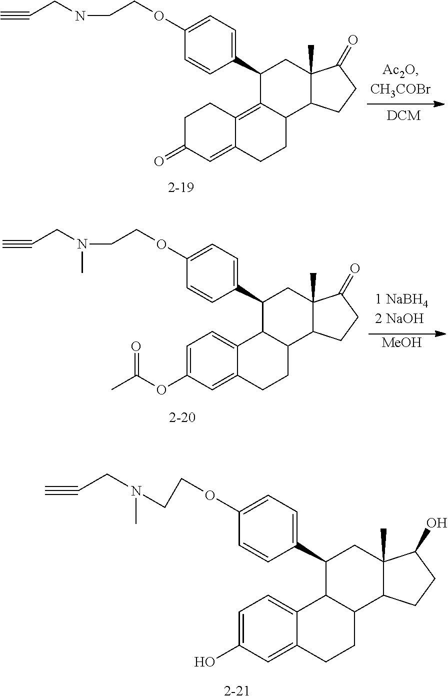

Thus, two 11β antiestrogens that were synthesized were 11β-(2-azidoethylphenoxy)-estra-1,3,5(10)-triene-3,17β-diol 2-13 and 11β-N-methyl-propargyl amine-(2-phenoxyethyl)-estra-1,3,5(10)-triene3,17β-diol 2-21.

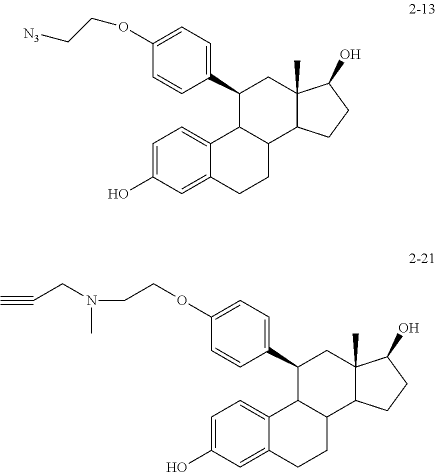

### Example 10

### Biological Evaluation of the 110 Antiestrogens

The ability of the described ERα ligands to bind to the ER was determined through their RBA against E2, in the competitive binding assay described above.

Ligand 2-13 demonstrated a high affinity with RBA values of 39.3±9.0 and 33.7±1.77 for ERα and ERβ respectively. These values demonstrate that 2-13 does not differentiate between ERα and ERβ, and therefore is a nonselective antiestrogen.

### Example 11

### Synthesis of Nitroxyls and Nitroxyl-Steroidal Hybrids

A propargyl radical was obtained according to Scheme 8

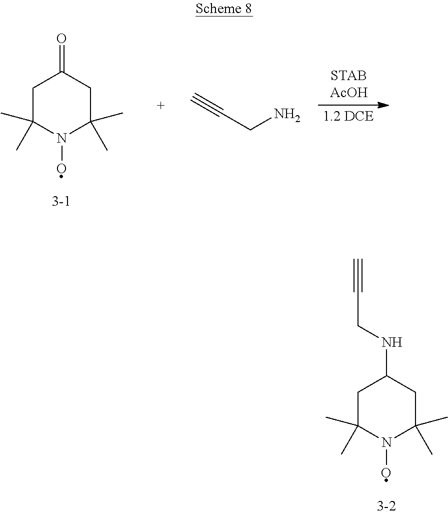

The radical was available to react with the complimentary azido antiestrogen.

A second nitroxide radical was prepared for SDSL of ERα in order to conduct the DEER experiments. SDSL requires a terminal iodo group (or similarly reactive leaving group) for easy attachment to the desired cysteine of ERα. Starting from 2,2,5,5-tetramethyl-3-pyrrolin-1-oxyl-3-ol, in situ formation of the mixed anhydride via ethyl chloroformate was followed by reduction with sodium borohydride. The resultant alcohol was then cleanly converted to the mesylate.

### Example 12

### Synthesis of 1-oxyl-2,2,5,5-tetramethyl-2,5-dihydropyrrol-3-ylmethane sulfonate

The synthesis of 3-6 proceeded according to Scheme 9.

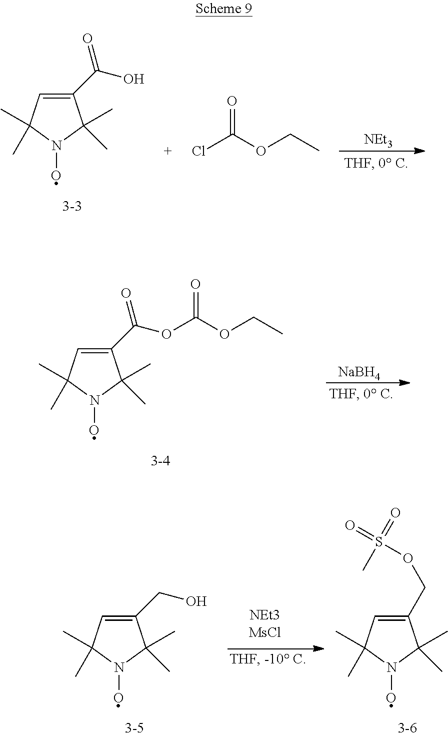

To a solution of the nitroxyl reagent 3-3 in THF and triethylamine at 0° C. was added a solution of ethyl chlorofommate (1 eq). The resultant triethylamine hydrochloride was removed by filtration and the reaction mixture evaporated to dryness. The crude product was redissolved in THF and cooled to 0° C. A suspension of sodium borohydride in THF was added and the reaction gently warmed to ambient temperature. The reaction was stirred for 2 h and the excess borohydride was decomposed with acetic acid. The crude product was purified by column chromatography on silica gel. The product 3-5 was dissolved in THF and triethylamine and cooled to −10° C. A cold solution of methanesulfonyl chloride in THF was added and the reaction stirred for 2 h. The reaction mixture was filtered to remove triethylamine hydrochloride and the product was purified by column chromatography on silica gel. The product was stored in the cold and dark.

### Example 13

Synthesis and Evaluation of ERα Dynamic Motion Probe

The ERα ESR probe was constructed from the complimentary azido antiestrogen 2-12 and the alkynyl TEMPO radical according to Scheme 10.

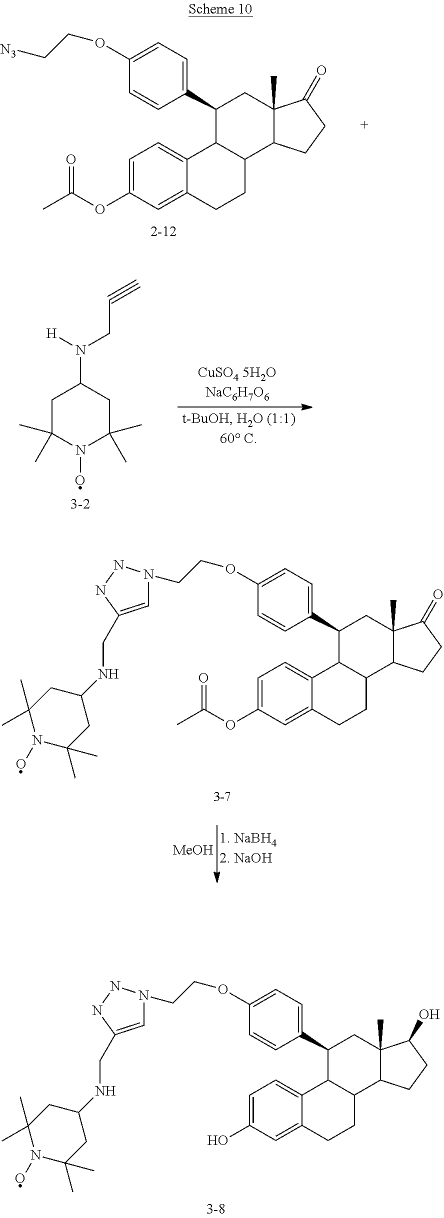

Synthesis of ERα Dynamic Motion Probe

Ligation of the azido-estradiol derivative 2-12 and the N-propargyl spin label 3-2 was achieved using the Huisgen [3+2] cycloaddition reaction. Reduction of the 17-ketone and saponification of the 3-acetate was accomplished in a one pot reaction, yielding the target spin probe 3-8 (two steps). The spin probe 3-8 was evaluated for its binding affinity and compared to its parent steroid 2-13, because if affinity is abolished this compound would not be a viable molecular probe Table 3.

TABLE 3

| Compound | RBA ERα | RBA ERβ | β/α |
|---|---|---|---|
| 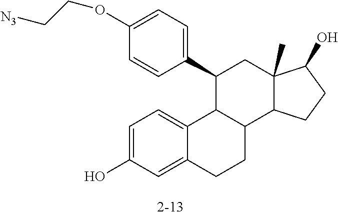 | 39.3 ± 9.0 | 33.7 ± 1.77 | 0.86 |
| 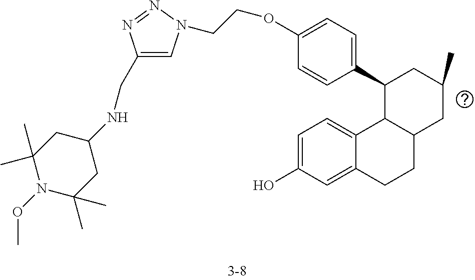 | 4.5 ± 1.17 | 1.9 ± 0.50 | 0.43 |

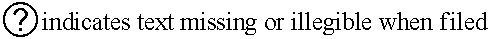

Incorporation of the nitroxide at the 11β-position reduced the affinity of 3-8 by less than an order of magnitude (4.5) for ERα, compared to the parent azidoethoxy steroid 2-13 (39.3). These results indicated that extension of the substituent at that position did not seriously compromise binding at the ligand binding pocket. The binding affinity for the receptor is probably reduced compared to the parent steroid because the resultant triazole introduces greater structural rigidity within the LBP. Further, at this position, the triazole reduces electrostatic interaction with Asp351 on the ERa-LBO. Similar to analogous steroidal derivatives, the new ligands show little ER-subtype selectivity.

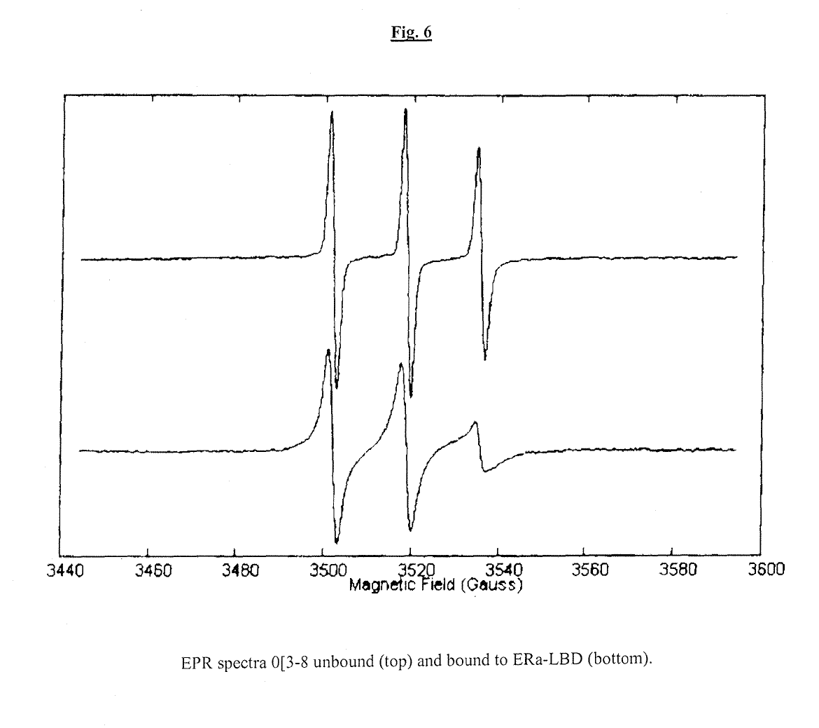

The EPR spectra of 3-8 unbound and bound to ERα-LBD is shown in FIG. 6.

An initial characterization of the binding of 3-8 to Era-LBD was carried out. FIG. 5 shows a stacked plot of the ESR spectra for 3-8 in solution (top) and in the presence of ER-LBD (bottom). The changes observed are characteristic of a decrease in rotation and an increase in the anisotropy of the rotation experienced by the nitroxide probe. This is consistent with the 3-8 binding to ER-LBD. Based on these preliminary results, 3-8 was selected as a molecular probe for ERα.

### Example 14

### Synthesis and Evaluation of a TKI-11β-Antiestrogen Hybrid

Erlotinib (Tarceva), Gefitinib (Iressa), Vandetanib-ZD 6474, and 3-9 are useful representative 4-anilinoquinazoline TKIs that can be ligated to a steroid component.

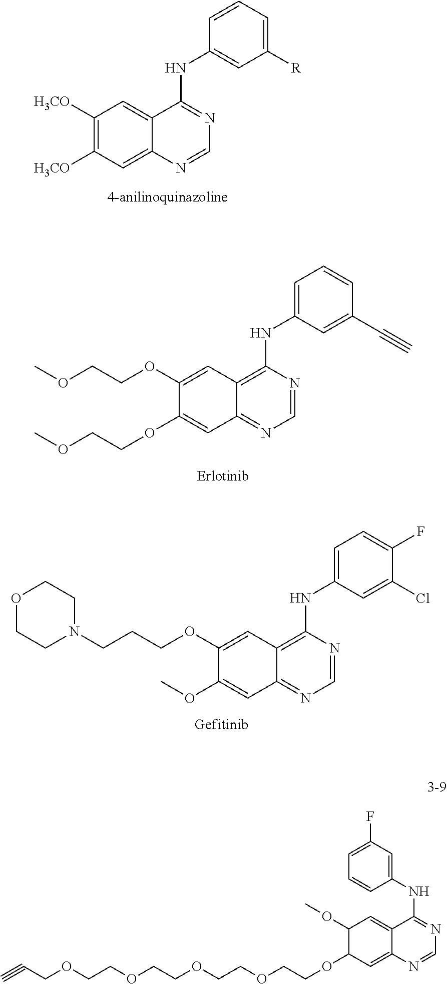

TKI 3-9 and its complimentary anti estrogen 2-12 were combined using a “click” reaction Scheme 11. The initial ligation of the anti-estrogen and TKI proceeded under the same conditions employed for the spin label probe. However, purification only removed the unreacted steroid, leaving a mixture of 3-9 and 3-10. Reduction and saponification of the mixture provided 3-11.

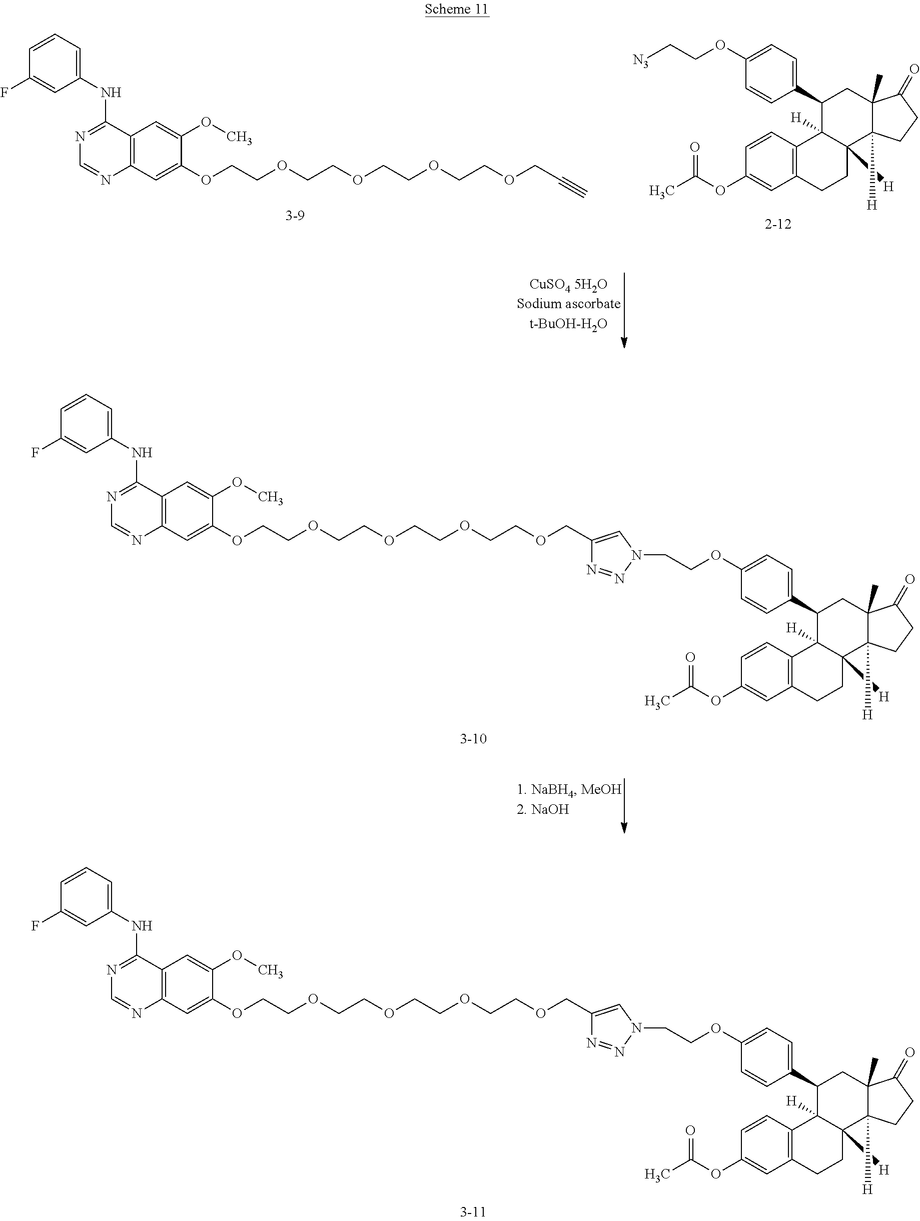

This compound is evaluated in a nonsmall cell lung cancer model that is responsive to both antihormonal and TKI therapy as described in Stabile, et al., Can. Res. (2005) 65:1459.

TKI derivatives and hybrids are also evaluated in an in vitro growth inhibition assay. The cells were maintained in Dulbecco's Modified Eagle's Medium (DMEM) (Sigma-Aldrich Inc., USA) supplemented with 10% fetal bovine serum (Sigma Chemical Co., USA) in a CO2 incubator. The cytotoxicity of the compounds was measured by MTT assay (T. Mossman, J. Immunol. Meth. (1983) 65:55-63). The cells were plated in a 96-ell plate at the density of 5000 cells/well (A431) and 8000 cells/well for MCF-7. After 24 h, cell culture media was replaced with DMEM containing 0.1% FBS and the cells were treated with different concentrations of the compounds (0.01-50 mM). The cells were later incubated for 72 h. The cytotoxicity was measured by adding 5 mg/mL of MTT (Sigma-Aldrich Inc., USA) to each well and incubated for another 3 h. The purple formazan crystals were dissolved by adding 100 mL of DMSO to each well. The absorbance was read at 570 nm in a spectrophotometer (Spectra Max 340). The cell death was calculated as follows:

Cell death=100−[(test absorbance/control absorbance)×100]

The test result is expressed as the concentration of a test compound which inhibits the cell growth by 50% (IC₅₀). (Chandregowda, et al., Europ. J. Median. Chem. (2009) 44:7 3046-3055.)

### Synthesis and Evaluation of an 11β-Antiestrogen-GDA Hybrid

Three 11βE2-GDA hybrids (3-13, 3-15, and 3-17) were synthesized between the two compounds and hybrid 3-17 was constructed via a “copper-less click” reaction.

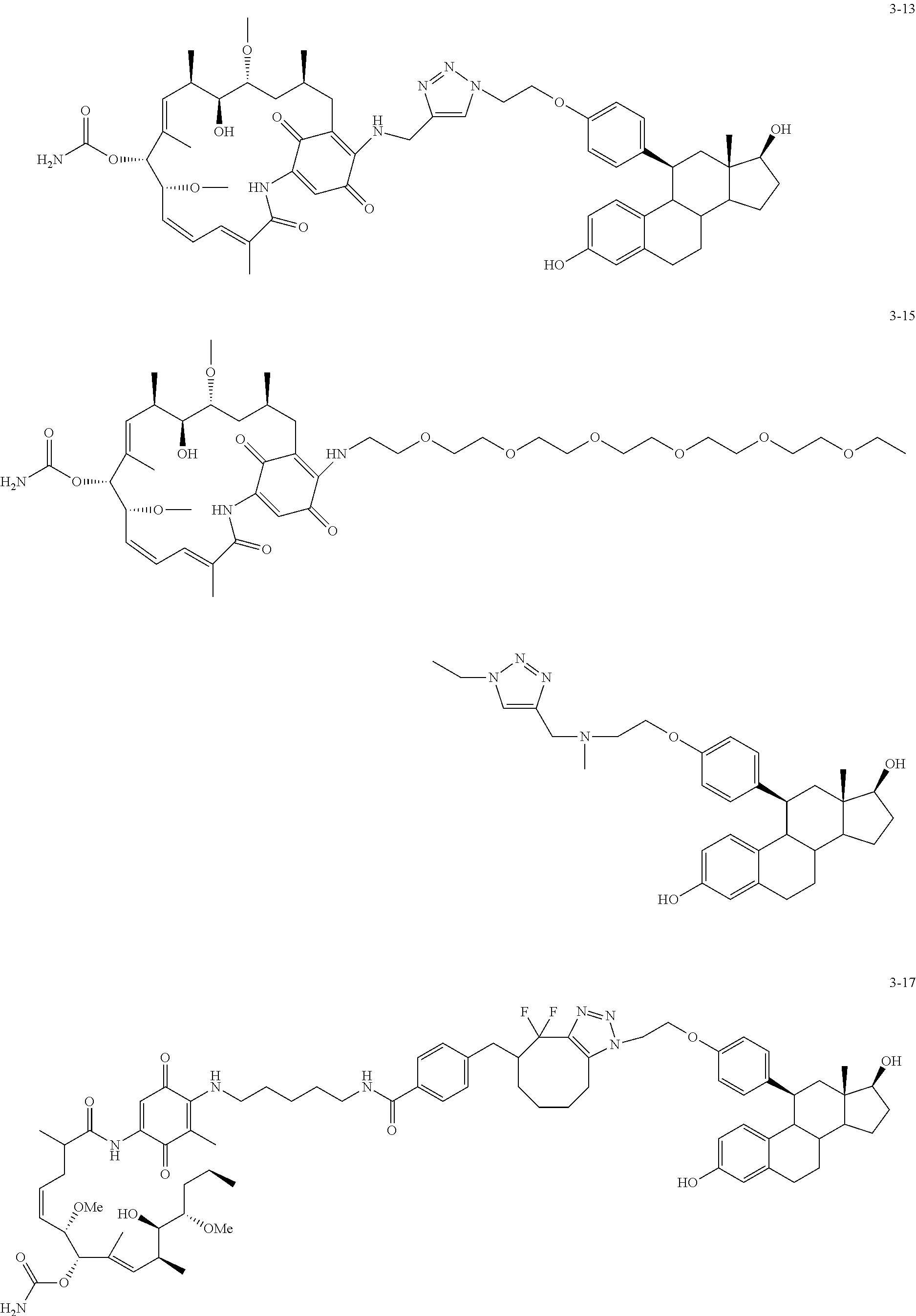

### 11β-anti-estrogen-GDA Hybrids

In this case, the fully formed estradiol core rather than the estrone-3-acetate was used. “Click” ligation using the standard copper catalyzed conditions was used to directly produce compounds 3-13 and 3-15 in 45.9% and 46.9% respectively. Hybrid 3-17 was prepared via a click reaction that was prepared without a copper catalyst. The length of the tethers was varied to study the relationship between the spatial proximity of the two entities and their effectiveness. Following purification, these hybrids were tested against two cell lines, MCF-7 an ER-positive breast cancer cell line and SKBr3 which overexpresses HER-2 but is ER-negative.

B. Anti-Proliferative Assay

MCF-7 and SKBr3 cells were maintained in a 1:1 mixture of Advanced DMEM/F12 (Gibco) supplemented with nonessential amino acids, L-glutamine (2 mM), streptomycin (500 g/mL), penicillin (100 units/mL), and 10% FBS. Cells were grown to confluence in a humidified atmosphere (37° C., 5% CO2), seeded (2000/well, 100 μL) in 96-well plates, and allowed to attach overnight. Compound or GDA at varying concentrations in DMSO (1% DMSO final concentration) was added, and cells were returned to the incubator for 72 h. At 72 h, the number of viable cells was determined using an MTS/PMS cell proliferation kit (Promega) per the manufacturer's instructions. Cells incubated in 1% DMSO were used as 100% proliferation, and values were adjusted accordingly. IC50 values were calculated from separate experiments performed in triplicate using GraphPad Prism. The results are shown in Table 3.

TABLE 3

| Compound | MCF-7 | | SKBr3 |
|---|---|---|---|
| 3-13 | 1.2 ± 0.09 | μM | 0.71 ± 0.16 μM |
| 3-15 | 102 ± 5.0 | nM | |
| GDA parent for 3-15 | 41 ± 5 | nM | |
| 3-17 | 17 | μM | |

All values are reported as EC⁵⁰

The data above demonstrate that the hybrids retain activity and begins to illustrate a trend regarding the GDA hybrids. There seems to be a correlation between the spatial proximity of the two compounds and their activity. Comparing hybrids 3-13 and 3-15 an order of magnitude improvement in its EC₅₀ can be seen for MCF-7 cells. This suggests that the longer tether, providing greater distance between the two compounds, is beneficial to their construction and efficacy, while the shorter chains can affect their activity, most likely due to spatial issues. The tether of compound 3-17, while an intermediate length, possesses different spatial, steric and physicochemical properties.

### Example 16

### Synthesis of 2-11(8S,11R,13S,14S)-11-(4-(2-azidoethoxy)phenyl)-1,2,7,8,12,13,15,16-octahydro-13-methyl-6H-cyclopenta [α]phenanthrene-3,17(11H,14H)-dione

The syntheses of 2-11 proceeded according to Scheme 12.

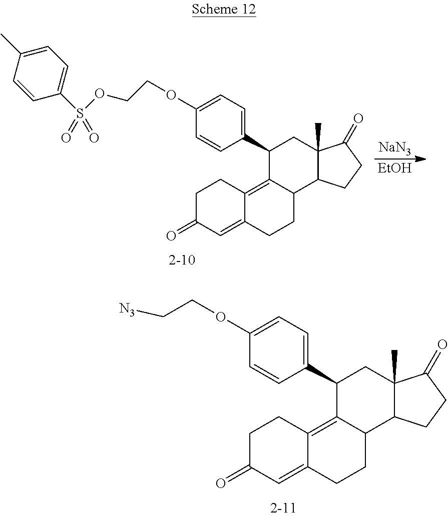

2-10 (80 mg, 0.143 mmol) was charged to a reaction tube and dissolved in ethanol (5 mL). Sodium azide (37.1 mg, 0.571 mmol) was added to the solution and the reaction was heated to 81° C. After 4 h, the reaction was poured into ice cold ethyl acetate (20 mL) and washed with water O×20 mL). Sodium chloride was added to the combined aqueous layers and then back extracted with ethyl acetate (20 L). The organic fractions were combined and dried over magnesium sulfate. The magnesium sulfate was filtered and the solvent removed under reduced pressure. Column chromatography yielded 2-11 (60 mg, 0.139 mmol, 97% yield).

¹H NMR (300 MHz, CDCl₃): δ 50.54 (s, 3H), 3.56 (t, 2H) 4.01 (t, 2H), 4.65 (d, J=6.9, 1H) 5.77 (s, 1H), 6.82 (d, J=8.8, 2H), 7.07 (d, J=8.8, 2H)

¹³C NMR (300 MHz, acetone-d6): δ 14.26, 21.80, 26.02, 27.07, 30.72, 35.07, 36.89, 38.14, 38.35, 39.74, 47.55, 50.32, 50.77, 67.25, 114.77, 122.96, 128.47, 130.00, 137.44, 145.32, 155.94, 156.79, 197.54, 217.44

IR: 2108.98 cm⁻¹

Rf (hexanes:ethyl acetate=4:6): 0.60

### Example 17

### Synthesis of 2-12 (8S,9R,11S,13S,14S)-11-(4-(2-azidoethoxy)phenyl)-7,8,9,11,12,13,14,15,16,17-decahydro-13-methyl-17-oxo-6H-cyclopenta[a]phenanthren-3-yl Acetate

The synthesis of 2-12 proceeded according to Scheme 13.

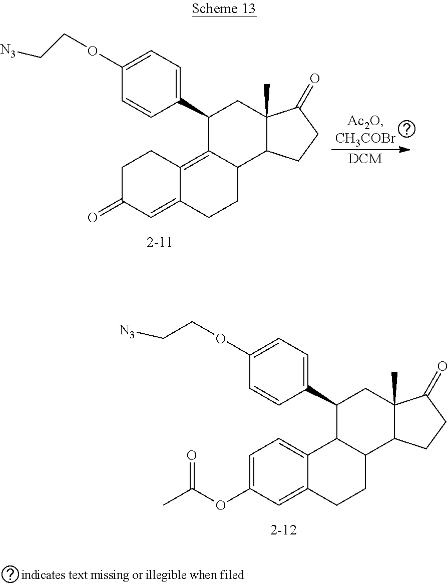

2-11 (19.0 mg, 0.044 mmol) was charged to a RBF and dissolved in DCM (3 mL). To this solution was added acetic anhydride (4.16 μL, 0.044 mmol) and acetyl bromide (8.2 μL, 0.110 mmol). After 4 hours the reaction mixture was poured into a solution of sodium bicarbonate (37.0 mg, 0.44 mmol) in water (15 L). The product was extracted with ethyl acetate (10 mL) and washed with water (3×15 mL). The aqueous washes were combined and back extracted with ethyl acetate (30 mL). The organic fractions were combined and dried over magnesium sulfate, which was removed by filtration and the solvent removed under reduced pressure leaving 2-12 (20 mg, 0.042 mmol, 96% yield).

¹H NMR (300 MHz, acetone-d6): δ 0.41 (s, 3H), 2.17 (s, 3H), 2.99 (m, 1H (11-H)), 3.59 (t, 2H) 4.10 (t, 2H), 6.63 (dd, 1H (2-H)), 6.70 (d, J=8.8, 2H), 6.87 (d, J=2.4, 1H (4-H)), 7.01 (d, J=8.5, 1H (1-H)), 7.09 (d, J=8.3, 2H)

¹³C NMR (500 MHz, acetone-d6): δ 15.00, 20.35, 21.27, 27.26, 29.96, 34.89, 35.18, 38.39, 40.46, 47.7, 48.00, 50.32, 52.14, 67.05, 113.87, 119.29, 121.90, 127.58, 130.93, 135.82, 136.31, 137.88, 148.75, 156.00, 168.96, 217.27

Rf (hexanes:ethyl acetate=6:4): 0.76

### Example 18

### Synthesis of 2-16 2-(N-methyl-N-(prop-2-ynyl)amino)ethanol

2-16 was synthesized according to Scheme 14.

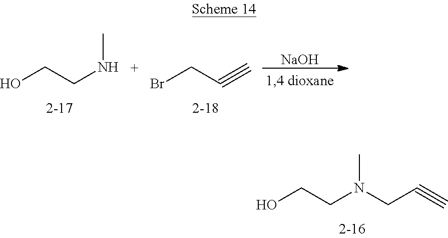

N sodium hydroxide (3.36 mL, 33.6 mmol) was added to a solution of 2-(methylamino)ethanol 2-17 (2.037 mL, 25.2 mmol) and propargyl bromide (in toluene) 2-18 (1.880 mL, 16.81 mmol) in anhydrous 1,4-dioxane (15 mL) at RT. After 13 hours the reaction was poured into a biphasic mixture of water (10 mL) and ethyl acetate (40 mL). The organic fraction was dried over magnesium sulfate, filtered, and the solvent removed under reduced pressure. The dark yellow/orange crude material was purified by column chromatography to afford 2-(methyl(prop-2-ynyl)amino)ethanol 2-16 (1.21 g, 10.71 mmol, 63.7% yield).

¹H NMR (300 MHz, CDCl₃): δ 2.14 (t, 1H), 2.18 (s, 3H), 2.44 (t, 2H), 3.22 (d, J=2.6, 2H), 3.46 (t, 2H), 3.65 (br. S, 1H)

¹³C NMR (300 MHz, CDCl₃): δ 41.52, 45.78, 57.40, 58.89, 73.71, 78.34

Rf (dichloromethane: methanol=9:1): 0.5

### Example 19

### Synthesis of 11β-N-methyl-propargyl amine-(2-phenoxyethyl)-3-metoxy-17-oxo-estra-1,3,5(10)-triene 2-19

The synthesis of 2-19 is shown in Scheme 15.

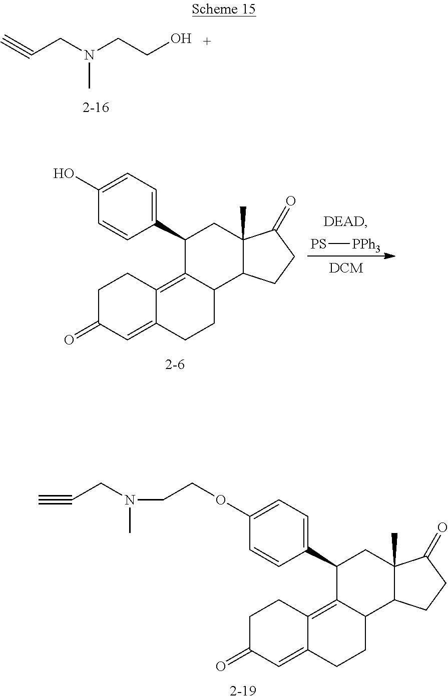

11β-(4-hydroxyphenyl)estradienedione 2-6 (56.0 mg, 0.154 mmol) and 2-(N-methyl-N-prop-2-yn-1-ylamino)ethanol 2-16 (20.98 mg, 0.185 mmol) were charged to a small reaction tube and dissolved in dichloromethane (5 mL). To this yellow/orange solution was added PS-triphenylphosphine (77 mg, 0.231 mmol) and the reaction was cooled to 0° C. After 15 min diethyl azodicarboxylate (0.044 mL, 0.278 mmol) was added and the reaction continued overnight, slowly warming up to 23° C. The polymer supported reagent was filtered off and washed with DCM (2×15 mL). The filtrate was washed with water (2×20 mL), after which all of the aqueous washes were combined; sodium chloride was added and then back extracted with 20 mL of DCM. Flash column chromatography yielded 2-19 (32 mg, 0.070 mmol, 45.3% yield). 16 mg of the starting material 2-6 was recovered making the adjusted yield 63.4%.

¹H NMR (300 MHz, acetone-d6): δ 0.54 (s, 3H), 2.34 (s, 3H), 2.70 (t, 1H), 3.4 (d, J=2.6, 2H), 4.05 (t, 2H), 4.48 (d, J=6.9, 1H), 5.67 (s, 1H), 6.87 (d, J=8.6, 2H), 7.20 (d, J=8.8, 2H)

¹³C NMR (300 MHz, acetone-d6): δ 14.21, 21.77, 26.01, 27.08, 30.69, 35.03, 36.86, 38.14, 38.34, 39.70, 41.77, 45.90, 47.53, 50.73, 54.50, 66.46, 73.96, 78.91, 114,65, 122.90, 128.34, 129.94, 136.85, 145.39, 155.93, 157.26, 197.50, 217.43

Rf (dichloromethane:ethyl acetate=1:1): 0.11

### Example 20

### Synthesis of 2-20 (8S,9R,11S,13S,14S)-11-(4-(2-([(2-propynyl]methylamino)ethoxy)phenyl)-7,8,9,11,12,13,14,15,16,17-decahydro-13-methyl-17-oxo-6H-cyclopenta[a]phenanthren-3-yl Acetate

The syntheses of 2-20 is shown in Scheme 16.

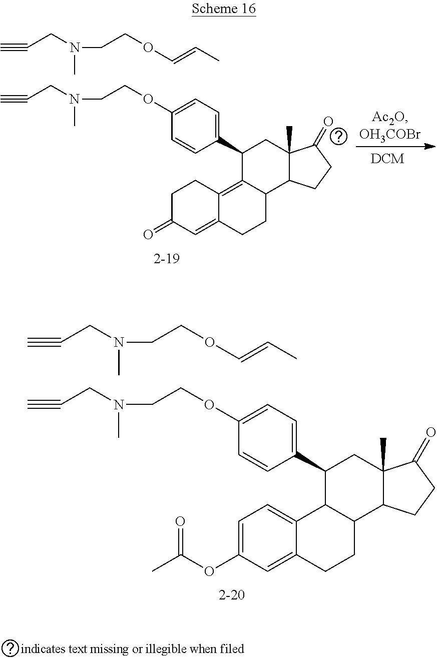

2-19 (27.0 mg, 0.059 mmol) was charged to a small reaction tube and dissolved in dichloromethane (4 mL). To this solution was added acetic anhydride (5.57 μL, 0.059 mmol) and acetyl bromide (10.9 μL, 0.148 mmol) at RT. The reaction continued overnight after which the product was extracted with ethyl acetate (20 mL) and washed with water (3×20 mL). The aqueous washes were combined; sodium chloride was added and then back extracted with ethyl acetate (20 mL). The organic fractions were combined and dried over magnesium sulfate, filtered, and the solvent removed under reduced pressure.

¹H NMR (300 MHz, CDCl₃): δ 0.44 (s, 3H), 2.24 (s, 3H), 2.40 (s, 3H), 2.84 (t, 1H) 3.44 (d, J=2.4, 2H), 3.97 (t, 2H), 6.64 (d, J=8.6, 1H), 6.86 (d, J=2.4, 2H), 6.95 (dd, 4H)

¹³C NMR (300 MHz, acetone-d6): δ 14.98, 20.351, 21.26, 27.25, 34.88, 35.15, 38.33, 40.44, 41.74, 45.87, 47.76, 47.98, 52.13, 54.49, 66.27, 73.92, 78.89, 113.79, 119.25, 121.85, 127.57, 130.78, 135.66, 135.83, 137.81, 148.70, 156.42, 168.93, 197.50, 217.26

Rf (dichloromethane: methanol=9:1): 0.69

### Example 21

### Synthesis of 2-13 (8S,9R,11S,13S,14S,17S)-11-(4-(2-azidoethoxy)phenyl)-7,8,9,11,12,13,14,15,16,17-decahydro-13-methyl-6H-cyclopenta[a]phenanthrene-3,17-diol

The syntheses of 2-13 proceeded according to Scheme 17.

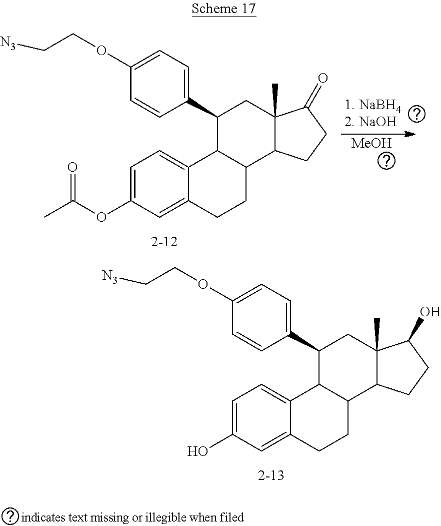

2-12 (29.0 mg, 0.06 mmol) was dissolved in methanol (5 mL). To this solution was added sodium borohydride (3.0 mg, 0.079 mmol). After 1 h 10 N sodium hydroxide (0.024 mL, 0.245 mmol) was added and the reaction continued for 16 h. The reaction was poured into an ice cold biphasic mixture of ethyl acetate (20 mL) and water (20 mL), after which the organic layer was washed with water (2×25 mL). The aqueous washes were combined, sodium chloride was added and then back extracted with ethyl acetate (30 mL). The organic layer was dried over magnesium sulfate. After filtration the solvent was adsorbed onto Florisil under reduced pressure. Flash chromatography yielded 2-13 (26 mg, 0.06 mmol, 98% yield).

¹H NMR (300 MHz, CDCl₃): δ 0.33 (s, 3H), 3.59 (t, 2H), 3.95 (m, 1H 11-H)), 4.09 (t, 2H), 6.37 (dd, 1H (2-H)), 6.55 (d, J=2.4, 2H), 6.67 (d, J=8.8, 2H (4-H)), 6.78 (d, J=8.8, 1H (1-H)), 7.06 (d, J=8.6, 1H),

¹³C NMR (500 MHz, acetone-d6): δ 12.98, 23.21, 28.29, 29.75, 30.12, 35.85, 38.70, 43.96, 45.99, 47.68, 50.34, 52.09, 67.02, 81.77, 113.25, 113.54, 115.27, 127.65, 128.12, 129.72, 130.21, 131.03, 137.25, 137.67, 154.00, 155.67

1R: 2086.03 cm¹,3351.50 cm¹

Rf (hexanes:ethyl acetate=6:4): 0.64

### Example 22

### Synthesis of 2-21(8S,9R,11S,13S,14S,17S)-11-(4-(2-(12-propynyl]methylamino)ethoxy)phenyl)-7,8,9,11,12,13,14,15,16,17-decahydro-13-methyl-6H-cyclopenta[a]-phenanthrene-3,17-diol

The syntheses of 2-21 is shown in Scheme 18.

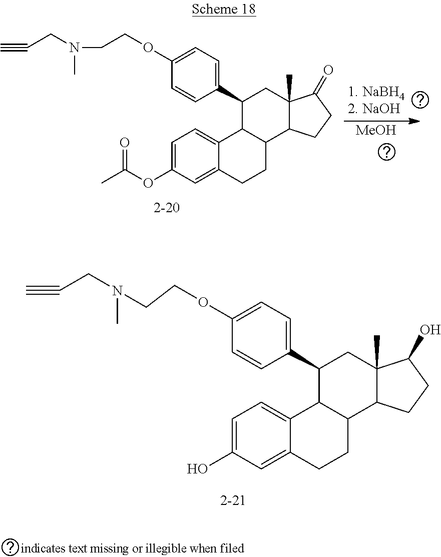

2-20 (50.0 mg, 0.100 mmol) was dissolved in methanol (5 mL). To this solution was added sodium borohydride (3.8 mg, 0.100 mmol). After 1 h 10 N sodium hydroxide (0.04 mL, 0.40 mmol) was added and the reaction continued overnight. The reaction was poured into an ice cold biphasic mixture of ethyl acetate (20 mL) and water (20 mL). The organic layer was washed with water (2×25 mL). The aqueous washes were combined and sodium chloride was added and then back extracted with ethyl acetate (30 mL). The organic layer was dried over magnesium sulfate. The magnesium sulfate was filtered and the organic material was adsorbed onto Florisil under reduced pressure. Column chromatography yielded 2-21 (30 mg, 0.065 mmol, 65.2% yield).

¹H NMR (500 MHz, CDCl₃): δ 0.37 (s, 3H), 2.48 (s, 3H), 2.84 (t, 1H) 3.44 (d, 2H), 3.97 (t, 2H), 6.39 (dd, 1H), 6.54 (d, J=2.7, 1H), 6.58 (d, J=8.8, 2H), 6.81 (d, J=8.3, 1H), 6.94 (d, J=8.3, 2H)

¹³C NMR (500 MHz, CDCl₃): δ 13.12, 19.10, 23.41, 28.22, 30.41, 30.67, 35.70, 38.52, 42.30, 43.89, 45.78, 47.56, 52.07, 54.48, 65.30, 68.30, 68.55, 74.63, 82.19, 82.82, 113.76, 114.67, 115.69, 127.84, 130.44, 130.79, 136.43, 137.83, 153.56, 155.66

Rf (DCM: methanol-9:1): 0.54

### Example 23

### Synthesis of Nitroxyl-Antiestrogen Hybrid 3-7

The syntheses of 3-2 proceeded according to Scheme 19.

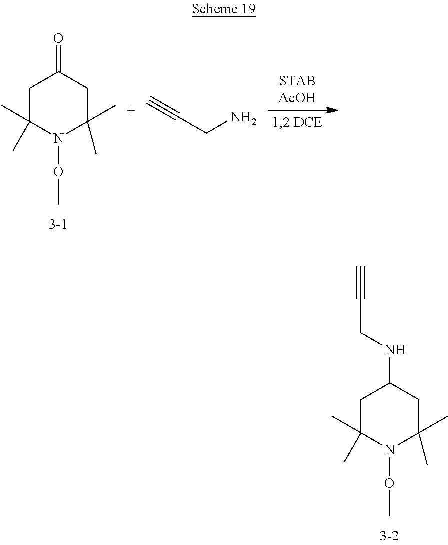

Tempone 3-1 (1.57 g, 9.22 mmol) and propargylamine (0.7 mL, 10.93 mmol) were dissolved in 1,2-dichloroethane (10 mL) to give a yellow solution. Acetic acid (0.67 mL, 10.95 mmols) was added causing an orange gelatinous precipitate. The reaction stirred under Argon for 24 h. Sodium triacetoxyborohydride (3.52 g, 16.6 mmol) was added neat and the reaction continued under Argon for 48 h after which the solvent was removed under reduced pressure leaving an orange oil. The oil was taken up in ethyl acetate (15 mL) and stirred at 40° C. for 1 h and then filtered. Florisil was added to the filtrate and the solvent removed under reduced pressure. Flash chromatography yielded 3-2 (1.542 g, 80%) as an orange solid.

Rf (ethyl acetate): 0.2

IR: 3400 cm⁻¹, 3300 cm⁻¹ and 3000 cm⁻¹

m.p.: 65-67° C.

The syntheses of 3-7 was carried out as shown Scheme 20.

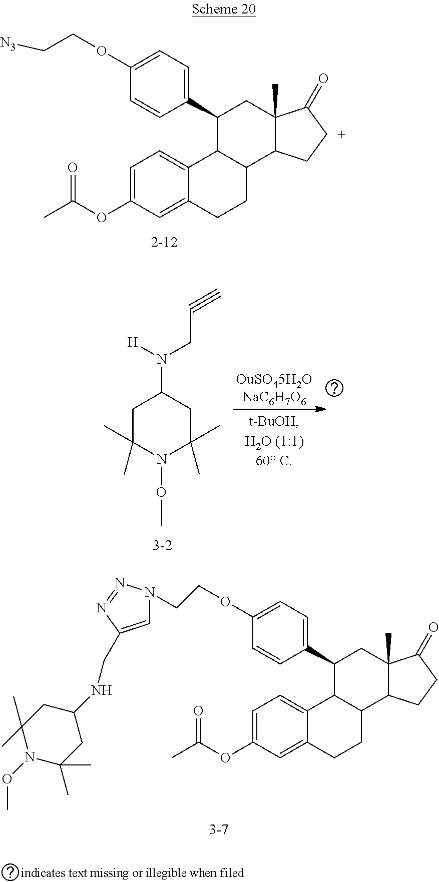

2-12 (100 mg, 0.21 mmol) and 2,2,6,6-Tetramethyl-4-(prop-2-ynylamino)-piperidone nitroxide 3-2 (75 mg, 0.36 mmol) were charged to a small vial and dissolved in a t-butanol (2 mL) and water (2 mL) mixture. To this vial was added copper(II) sulfate pentahydrate (0.05 mL, 2.1 μmol) and (+)-sodium L-ascorbate (0.21 mL, 10.56 μmol). The reaction proceeded overnight at 60° C. The following day the reaction was stopped by being poured into an ice cold biphasic mixture of ethyl acetate and water. The organic layer was washed 2× more with water (25 mL). All of the aqueous washes were combined and back extracted with ethyl acetate and sodium chloride. The organic layer was dried over magnesium sulfate, filtered and concentrated. Purification by column chromatography yielded 3-7 (110 mg, 76% yield)

MS: m/z calculated 682.4, M⁺² observed 684.4

Rf (dichloromethane: methanol=9:1): 0.66

### Example 24

### Synthesis of Nitroxyl-Anti-Estrogen Hybrid 3-8

The syntheses of 3-8 proceeded according to Scheme 21.

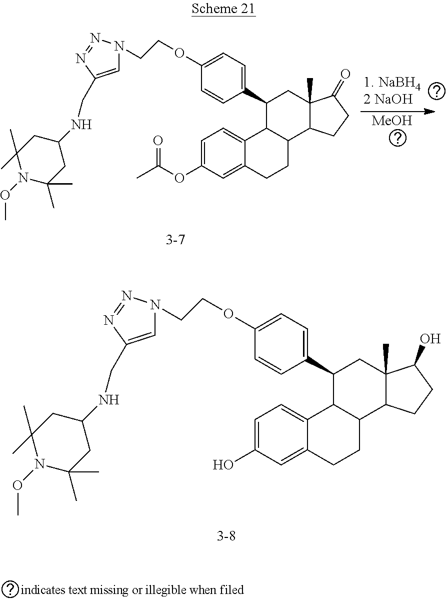

3-7 (75 mg, 0.11 mmol) was dissolved in methanol (2 mL). To this orange solution was added sodium borohydride (4.66 μL, 0.13 mmol). After 2 h 10 N sodium hydroxide (0.044 mL, 0.44 mmol) solution was added and the reaction continued for another 2 h. The reaction was poured into a biphasic mixture of ethyl acetate (20 mL) and water (20 mL). The organic layer was washed with water (2×20 mL). Sodium chloride was added to the combined aqueous layers and back extracted with ethyl acetate (20 mL). The organic layers were combined and dried over magnesium sulfate, filtered and the solvent removed under reduced pressure. Column chromatography yielded 3-8 (65 mg, 0.10 mmol, 92% yield).

HRMS: m/z calculated 642.402, M⁺¹ observed 643.414

Rf (dichloromethane:methanol=95:5): 0.14

### Example 25

### Synthesis of 3-10 (8S,9R,11S,13S,14S)-11-(4-(2-(4((2-(2-(2-(2-(4-(3-fluorophenylamino)-6-methoxyquinazolin-7-yloxy)ethoxy)ethoxy)ethoxy)ethoxy)methyl)-1H-1,2,3-triazol-1-yl)ethoxy)phenyl)-7,8,9,11,12,13,14,15,16,17-decahydro-13-methyl-17-oxo-6H-cyclopenta[a]phenanthren-3-yl acetate

The syntheses of 3-10 is in Scheme 22.

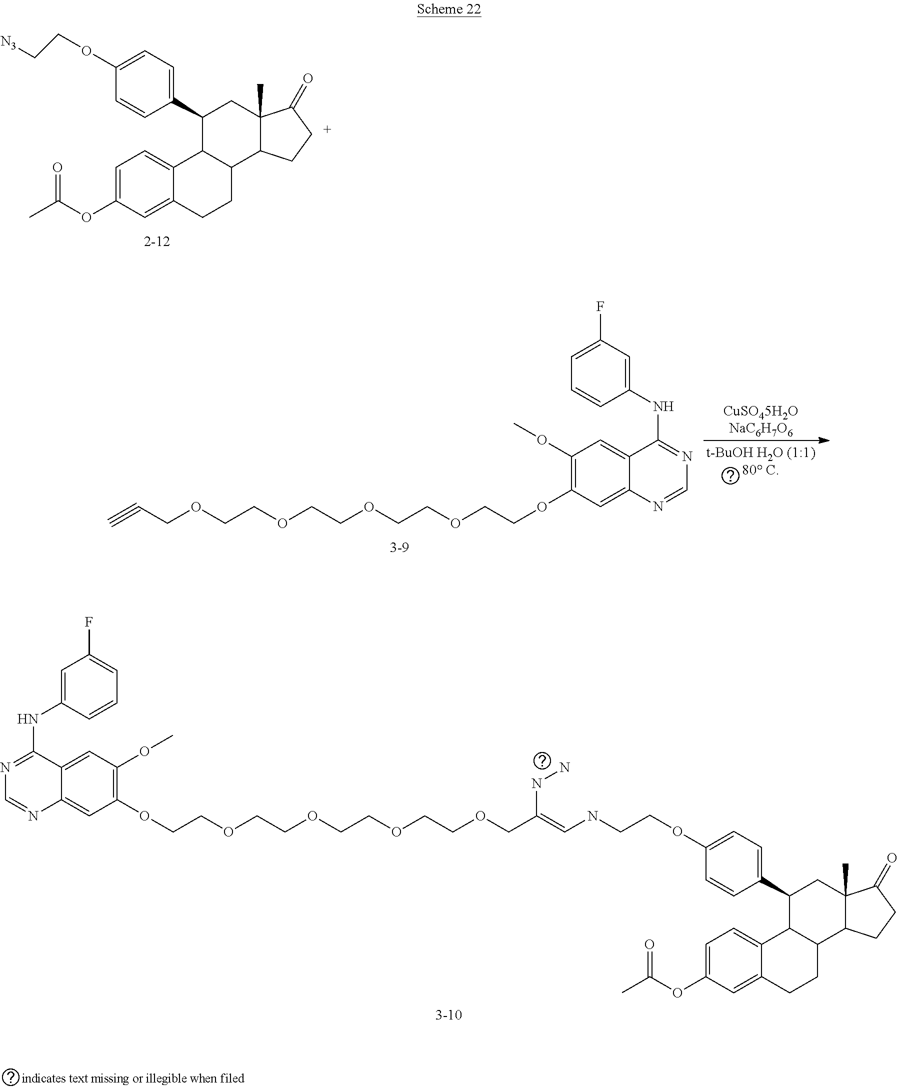

2-12 (15.0 mg, 0.032 mmol) and 3-9 (15.0 mg, 0.032 mmol) were charged to a round bottom and dissolved in a mixture of water (2 raL) and t-butanol (2 mL). To this solution copper (II) sulfate pentahydrate (7.92 μL, 0.32 mmol) and (+)-sodium L-ascorbate (31.0 μL, 1.58 umol) were added and the reaction was heated to 80° C. After 18 h the reaction was cooled to RT and poured into a biphasic mixture of water (15 mL) and ethyl acetate (15 mL). The organic layer was washed with water (2×15 mL) and then all of the aqueous layers were combined, sodium chloride was added and then back extracted with ethyl acetate (20 mL). The organic fractions were combined and dried over magnesium sulfate, filtered and concentrated. Column chromatography was performed to remove unreacted 2-12 before the next step, leaving a mixture of 3-9 and 3-10.

### Example 26

### Synthesis of 3-11: (8S,9R,11S,13S,14S)-11-(4-(2-(4-((2-(2-(2-(2-(4-(3-fluorophenylamino)-6-methoxyquinazolin-7-yloxy)ethoxy)ethoxy)ethoxy)ethoxy)methyl)-1H-1,2,3-triazol-1-yl)ethoxy)phenyl)-7,8,9,11,12,13,14,15,16,17-decahydro-13-methyl-17-oxo-6H-cyclopenta[a]phenanthren-3-yl Acetate

The syntheses of 3-11 is shown in Scheme 23.

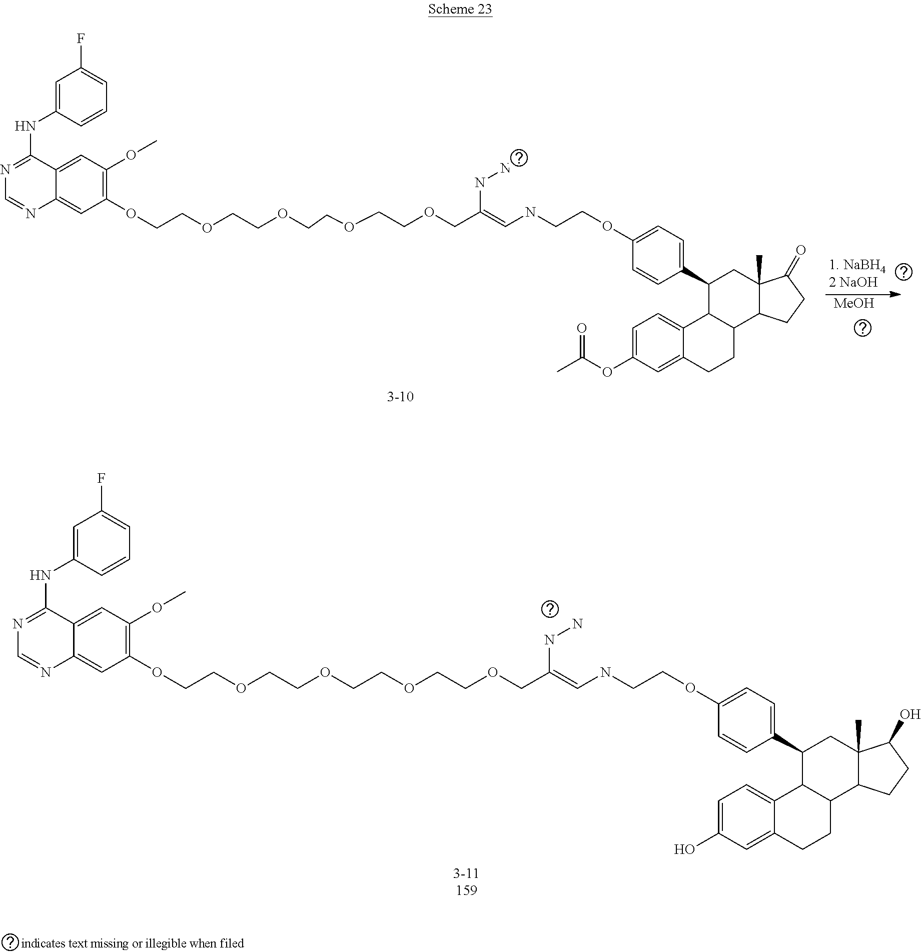

3-9 and 3-10 (26.0 mg) was dissolved in methanol (5 mL) and sodium borohydride (1.09 mg, 0.029 mmol) was added. After 3 h 10 N sodium hydroxide (9.62 μL, 0.096 mmol) was added the reaction left overnight. In the morning the reaction was poured into a biphasic mixture of water (15 mL) and ethyl acetate (15 mL). The organic layer was washed with water (2×15 mL) and then all of the aqueous layers were combined, sodium chloride was added and then back extracted with ethyl acetate (20 mL). The organic fractions were combined and dried over magnesium sulfate, filtered and concentrated. Column chromatography yielded 3-11 (9 mg, 9.65 mmol, 32.2% yield for all three steps).

¹H NMR (500 MHz, acetone d-6): δ 0.32 (s, 3H), 3.55 (s, 12H), 4.30 (t, 2H), 4.55 (s, 3H), 4.72 (t, 2H), 6.37 (dd, 1H (2-H)), 6.56 (d, J=2.6, 1H), 6.63 (d, J=8.9, 2H (4-H)), 6.74 (d, J=8.4 1H), 7.00 (d, J=8.5, 2H) 7.24 (s, 1H), 7.36 (q, 1H), 7.59 (d, J=8.3, 1H), 7.74 (s, 1H), 7.96 (s, 1H), 8.05 (d, J−10.1, 1H) 8.58 (s, 1H), 9.09 (s, 1H)

¹³C NMR (500 MHz, acetone d-6): δ 13.02, 23.19, 28.26, 29.75, 30.27, 35.83, 38.67, 43.95, 45.95, 47.63, 49.62, 52.04, 56.05, 64.34, 66.47, 68.73, 69.34, 69.61, 70.51, 70.56, 70.65, 72.34, 81.88, 101.40, 108.62, 109.28, 109.46, 113.37, 113.61, 115.34, 117.01, 124.05, 127.63, 129.63, 129.92, 131.02, 137.42, 137.63, 145.05, 147.92, 150.14, 153.00, 154.53, 155.45, 163.96

HRMS (FAB): m/z calculated 932.448, M⁺¹ observed 933.456

Rf (dichloromethane: methanol=9:1): 0.44

### Example 27

### Synthesis of Geldanamycin-aminomethyl-triazolo-ethoxy Anti-Estrogen 3-13

The syntheses of 3-13 is shown in Scheme 24.

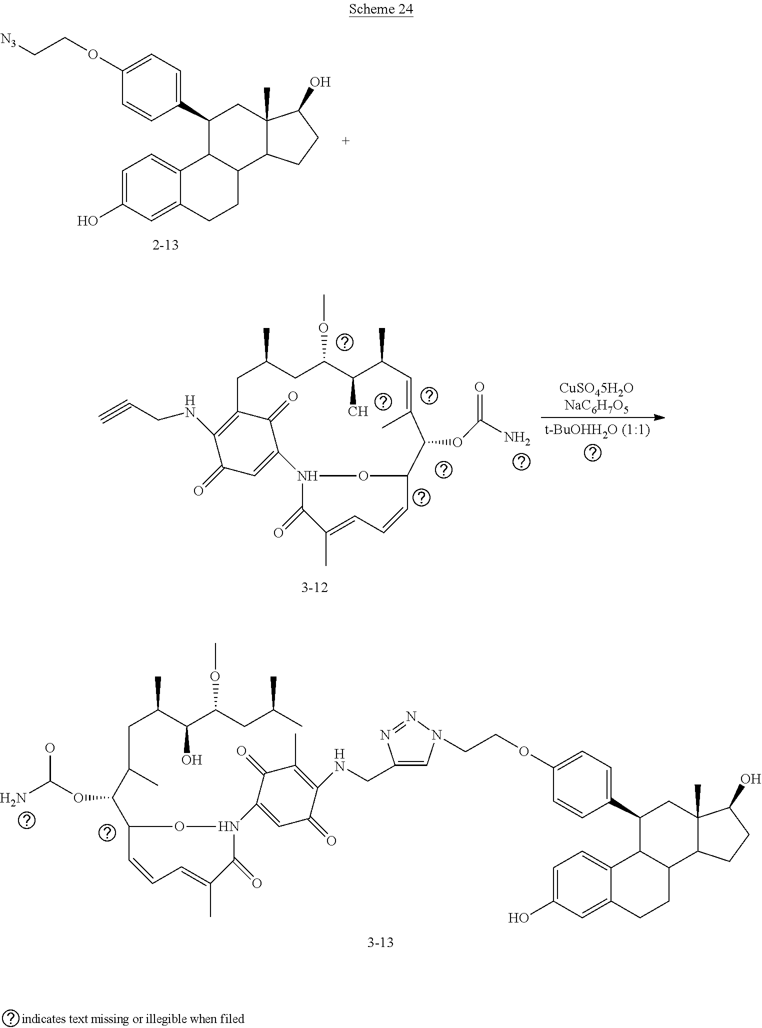

2-13 (5.00 mg, 0.012 mmol) was charged to a RBF and t-butanol (1 mL) and water (1 mL) was added. To this was added 3-12 (5.00 mg, 8.57 μmol), which was dissolved in a 1:1 mixture of t-butanol and water (2 mL). The solution instantly turned purple. Copper (II) sulfate pentahydrate (120 μL, 4.80 μmol) and (+)-sodium L-ascorbate (8.48 μL, 0.428 μmol) were added to this solution. The reaction continued for 14 h, after which it was poured into a biphasic mixture of water (10 mL) and ethyl acetate (10 mL). The organic layer was washed with water (2×15 mL) and then all of the aqueous layers were combined, sodium chloride was added and then back extracted with ethyl acetate (15 mL). The organic fractions were combined and dried over magnesium sulfate, filtered and concentrated leaving a purple solid on the wall of the RBF. Column chromatography afforded 3-13 (4 mg, 3.93 μmol, 45.9%).

¹H NMR (500 MHz, acetone d-6): δ 0.29 (s, 3H), 0.89 (d, J=6.9, 1H), 1.04 (d, J=6.6, 1H), 1.19 (s, 3H), 1.78 (s, 3H), 2.82 (s, 3H), 3.21 (s, 3H), 3.31 (s), 4.32 (t, 2H), 4.54 (d, J=9.5, 1H), 4.78 (t, 2H), 4.92 (d, J=5.8, 1H), 5.11 (s, 1H), 5.79 (d, J=9.8, 1H), 5.85 (t, 1H), 6.37 (d, J=8.2, 1H), 6.55 (s, 1H), 6.65 (t, 1H), 6.76 (d, J=8.4, 1H), 7.02 (d, J=8.3, 1H), 7.14 (s, 1H). 7.30 (d, J=11.4, 1H), 7.87 (s, 1H), 8.01 (s, 1H), 9.40 (s, 1H)

HRMS (FAB): m/z calculated 1016.5259, M⁺¹ observed 1017.5331

Rf (dichloromethane: methanol=95:5): 0.14

### Example 28

### Synthesis of Geldanamycin-aminoheptaethylene glycolyl-triazolo-methylaminoethoxy Anti-Estrogen 3-15

The syntheses of 3-15 is shown in Scheme 25.

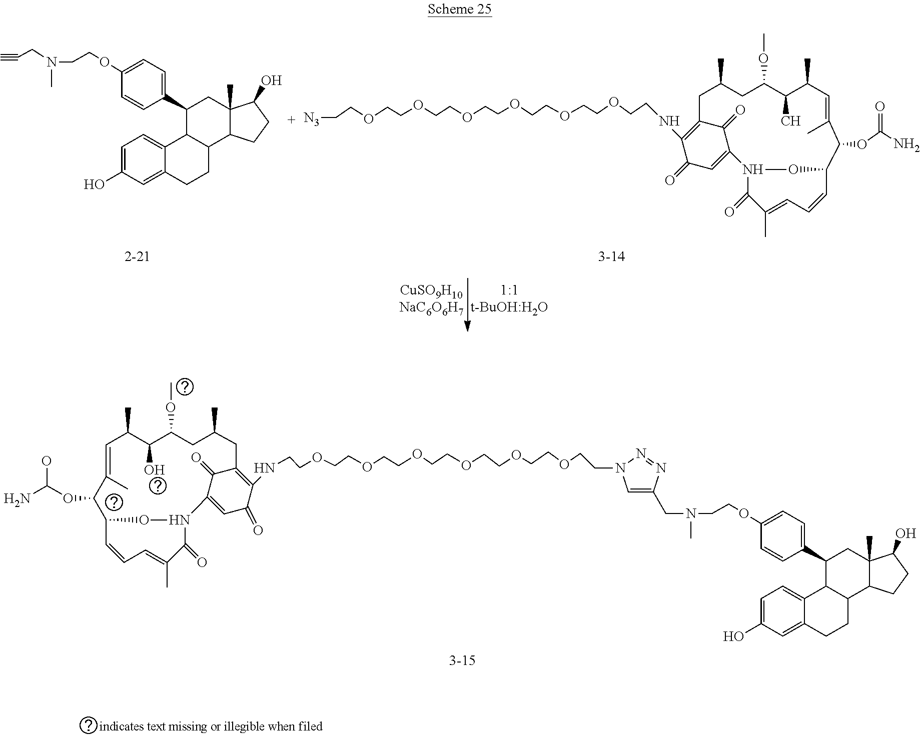

2-21 (5.49 mg, 0.012 mmol) was charged to a small scintillation vial and t-butanol (1 mL) and water (1 mL) were added. To this 3-14 (7.00 mg, 7.96 μmol) was added, dissolved in a mixture of t-butanol (1 mL) and water (1 mL). The solution instantly turned purple. To this solution copper (II) sulfate pentahydrate (1.991 μL, 0.080 μmol) and (+)-sodium L-ascorbate (7.88 μL, 0.398 μmol) were added. After 18 h the reaction was poured into a biphasic mixture of water (10 mL) and ethyl acetate (10 mL). The organic layer was washed with water (2×15 mL) and then all of the aqueous layers were combined, sodium chloride was added and then back extracted with ethyl acetate (15 mL). The organic fractions were combined and dried over magnesium sulfate, filtered and concentrated leaving a purple solid. Column chromatography yielded. 3-15 (5 mg, 3.74 μmol, 46.9% yield)

¹H NMR (500 MHz, CDCl₃): δ 0.33 (s, 3H), 0.93 (d, J=6.9, 1H), 1.08 (d, J=6.6, 1H), 1.19 (s, 3H), 1.78 (s, 3H), 2.10 (s, 3H), 2.78 (d, J=4.6, 1H), 3.16 (s, 3,20 (s, 3H), 3.29 (s, 3H), 3.32 (s, 3H), 3.42 (s, 3H), 3.56 (m, 18), 4.32 (t, 2H), 4.54 (d, J=9.5, 1H), 4.78 (t, 2H), 4.92 (d, J=5.8, 1H), 5.13 (s, 1H), 5.79 (d, J=9.8, 1H), 6.37 (d, J=8.2, 1H), 6.55 (s, 1H), 6.65 (t, 1H), 6.76 (d, J=8.4, 1H), 7.02 (d, J=8.3, 1H), 7.14 (s, 1H), 7.30 (d, J=11.4, 1H), 7.87 (s, 1H), 8.01 (s, 1H), 9.40 (s, 1H)

HRMS (FAB): m/z calculated 1337.7410, M⁺¹ observed 1338.7489

Rf (dichloromethane: methanol=9:1): 0.57

### Example 29

### Synthesis of Geldanamycin aminopentanamidocarbonylphenyl-methyl difluorocyclooctenyltriazolo-ethoxy Anti-Estrogen 3-17

The syntheses of 3-17 is shown in Scheme 26.

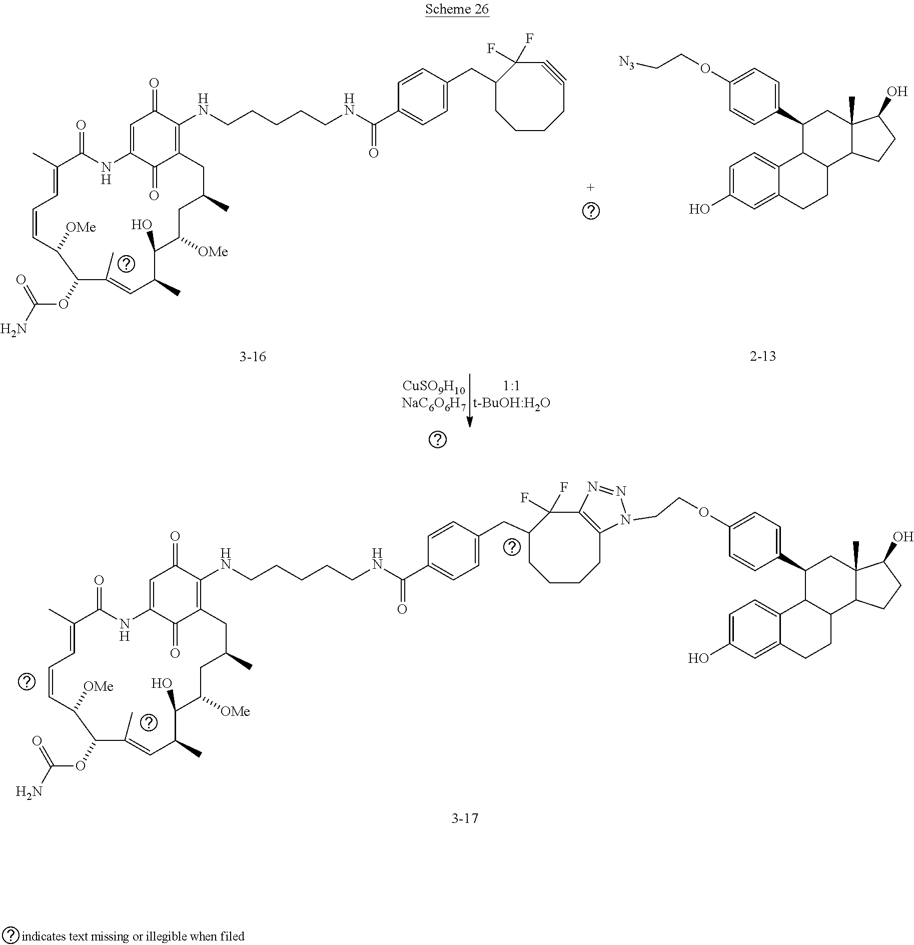

Geldanamycin derivative 3-16 (6.00 mg, 6.73 mmol) and 2-13 (3.20 mg, 7.38 μmol) were dissolved in t-butanol (1 mL) and water (1 mL) mixture. The reaction proceeded for 24 h and was poured into a biphasic mixture of water (10 mL) and ethyl acetate (10 mL). The organic layer was washed with water (2×10 mL) and all of the aqueous layers were combined, sodium chloride was added and then back extracted with ethyl acetate (15 mL). The organic fractions were combined and dried over magnesium sulfate, filtered and the solvent removed under reduced pressure. Column chromatography yielded Product 1 (4.5 mg, 4.91 μmol, 50.5% yield).

¹H NMR (500 MHz, CDCl3)δ0.29 (s, 3H), 0.98 (s, 3H), 1.00 (s, 3H), 1.25 (s, 3H), 1.78 (s, 3H), 2.23 (s, 1H), 2.63 (s, 3H), 3.26 (s, 3H), 3.35 (s, 3H), 4.32 (d, J=9.76, 2H), 5.19 (s, 1H), 5.29 (s, 1H), 5.82 (s, 2H), 5.88 (d, J=11.18, 1H), 6,48 (d, J=8.33, 1H), 6.57 (t, 2H), 7.32 (s, 2H), 7.67 (d, J=8.0, 1H), 8.00 (s, 2H), 9.17 (s, 1H)

HRMS (FAB): m/z calculated 1323.7007, M⁺¹ observed 1324.7818

Rf (dichloromethane: methanol=9:1): 0.48

### EQUIVALENTS

Those skilled in the art will recognize, or be able to ascertain, using no more than routine experimentation, numerous equivalents to the specific embodiments described specifically in this disclosure. Such equivalents are intended to be encompassed in the scope of the following claims.

1. A compound of Formula (Ia),
S′-A′ Formula (Ia)
wherein:
S′ is S-L′, where S is an 11β-substituted steroid; A′ is A-L″, where A is a quinone antibiotic; and L′ and L″ are each a half-linker that together form L, a linker.

2. The compound of claim 1, wherein A comprises a compound of the formula: 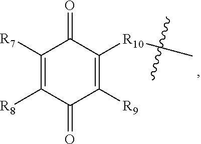
wherein
R₇, R₈, and R₉ are each independently H, alkyl, cycloalkyl, aralkyl, alkoxy, aryl, heterocycle, amine, or halogen; and
R₁₀ is C, S, N, or O.

3. The compound of claim 1, wherein A is mitomycin C.

4. The compound of claim 1, wherein A is geldanamycin.

5. The compound of claim 1, wherein S′ is a compound having of Formula (III), 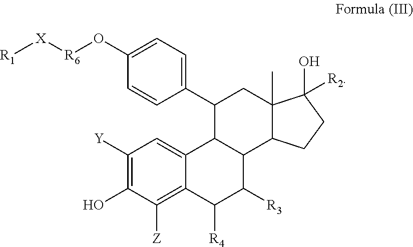
wherein:
R₁ is an oligoethylene glycol having from 1-10 units;
R₂ is H, ethenyl, ethynyl, haloethenyl, alkylthioethenyl, alkylselenoethenyl, arylthioethenyl, arylselenoethenyl, or aryl vinyl wherein the arylvinyl may have up to four substituents and the substituents may be alkyl, aryl, or fluoroalkyl;
R₃ is a C₁-C₆-alkyl, aralkyl, hydroxyl, ketone, or ether;
R₄ is a C₂-C₆-alkyl, aralkyl, hydroxyl, ketone, or ether;
R₆ is an alkyl or cycloalkyl;
X is O, NH, N-alkyl, N-cycloalkyl, N-aralkyl, or S; and
Y and Z are each independently H, F, Cl, Br, I, C₁-C₆-alkyl, hydroxyalkyl, alkoxyalkyl, NO₂, or NH₂.

6. The compound of claim 1, wherein S is anti-estrogen steroid.

7. The compound of claim 5, wherein the anti-estrogen steroid is estradiol.

8. The compound of claim 1, wherein S is anti-androgen steroid.

9. The compound of claim 1, wherein S is anti-progestin steroid.

10. The compound of claim 1, wherein S is anti-glucocorticoid steroid.

11. The compound of claim 1, wherein L is an oligoethylene glycol, a polymethylene group, a polyamide, or combinations thereof.

12. The compound of claim 1, wherein the compound is [chemical structure]
